(19) United States
(12) Patent Application Publication
(10) Pub. No.: US 2012/0089191 A1
(43) Pub. Date: Apr. 12, 2012

(54) METHODS FOR STABILIZING BONE STRUCTURES

(75) Inventors: Moti Altarac, Irvine, CA (US); Joey Carnia Reglos, Lake Forest, CA (US); Stanley Kyle Hayes, Mission Viejo, CA (US); Jean Harnapp, Irvine, CA (US); Daniel H. Kim, Los Altos, CA (US); J. Christopher Flaherty, Topsilog, MA (US)

(73) Assignee: Exactech, Inc.

(21) Appl. No.: 12/966,790

(22) Filed: Dec. 13, 2010

Related U.S. Application Data
(62) Division of application No. 11/586,849, filed on Oct. 25, 2006.
Application No. 11/586,849 is a continuation-in-part of application No. 11/362,366, filed on Feb. 23, 2006.
(60) Provisional application No. 60/701,660, filed on Jul. 22, 2005.

(51) Int. Cl. A61B 17/88

(52) U.S. Cl. 606/279

(57) ABSTRACT

Methods, systems, devices and tools for placing bone stabilization components in a patient are provided. The systems and devices have a reduced number of discrete components that allow placement through small incisions and tubes. More particularly, the present invention is directed to systems and methods of treating the spine, which eliminate pain and enable spinal motion, which effectively mimics that of a normally functioning spine. Methods are also provided for stabilizing the spine and for implanting the subject systems.

### STATEMENT OF RELATED APPLICATIONS

This application is a divisional of U.S. patent application Ser. No. 11/586,849 filed Oct. 25, 2006, entitled “Systems and Methods for Stabilization of Bone Structures,” which is a continuation-in-part of co-pending U.S. patent application Ser. No. 11/362,366, filed Feb. 23, 2006, entitled “Systems And Methods For Stabilization of Bone Structures,” which claims priority to U.S. Patent Application Ser. No. 60/701,660, filed on Jul. 22, 2005 entitled “Systems and methods for stabilization of bone structures,” all of which are incorporated herein by reference in their entirety.

The present invention generally relates to surgical instruments and methods for using these instruments. More particularly, but not exclusively, minimally invasive methods of stabilizing one or more bone structures is disclosed.

### BACKGROUND OF THE INVENTION

### Field of the Invention

Systems, methods and devices for stabilizing one or more bone structures of a patient have been available for many years. Securing a metal plate is used to stabilize a broken bone, maintaining the bone in a desired position during the healing process. These implanted plates are either removed when sufficient healing has occurred or left in place for a long-term or indefinite, chronic period. A procedure involving the placement of one or more elongated rods extending between two bone structures or between two components of a single bone structure is often used as a stabilization technique. These rods are placed alongside the bone structure or structures and attached to bone via one or more attachment mechanisms (e.g. bone screws, anchors, etc). These procedures typically require large incisions and also significant tissue manipulation to adequately expose the areas intended for the attachment. The procedures are associated with long recovery times and increased potential for adverse events, such as infection, muscle and other tissue trauma and scarring.

Currently available minimally invasive techniques and products are limited. These procedures are difficult to perform, especially in spinal applications in which the attachment points are deeper in tissue, and damage to neighboring tissue must be avoided. Many of the currently available less invasive products remain somewhat invasive due to component configurations, and required manipulations to be performed during the attachment.

In reference specifically to treatment of the spine, FIG. 1A illustrates a portion of the human spine having a superior vertebra 2 and an inferior vertebra 4, with an intervertebral disc 6 located in between the two vertebral bodies. The superior vertebra 2 has superior facet joints 8a and 8b, inferior facet joints 10a and 10b, posterior arch 16 and spinous process 18. Pedicles 3a and 3b interconnect the respective superior facet joints 8a, 8b to the vertebral body 2. Extending laterally from superior facet joints 8a, 8b are transverse processes 7a and 7b, respectively. Extending between each inferior facet joints 10a and 10b and the spinous process 18 are lamina 5a and 5b, respectively. Similarly, inferior vertebra 4 has superior facet joints 12a and 12b, superior pedicles 9a and 9b, transverse processes 11a and 11b, inferior facet joints 14a and 14b, lamina 15a and 15b, posterior arch 20, spinous process 22.

The superior vertebra with its inferior facets, the inferior vertebra with its superior facets, the intervertebral disc, and seven spinal ligaments (not shown) extending between the superior and inferior vertebrae together comprise a spinal motion segment or functional spine unit. Each spinal motion segment enables motion along three orthogonal axis, both in rotation and in translation. The various spinal motions are illustrated in FIGS. 2A-C. In particular, FIG. 2A illustrates flexion and extension motions and axial loading, FIG. 2B illustrates lateral bending motion and FIG. 2C illustrates axial rotational motion. A normally functioning spinal motion segment provides physiological limits and stiffness in each rotational and translational direction to create a stable and strong column structure to support physiological loads.

Traumatic, inflammatory, metabolic, synovial, neoplastic and degenerative disorders of the spine can produce debilitating pain that can affect a spinal motion segment's ability to properly function. The specific location or source of spinal pain is most often an affected intervertebral disc or facet joint. Often, a disorder in one location or spinal component can lead to eventual deterioration or disorder, and ultimately, pain in the other.

Spine fusion (arthrodesis) is a procedure in which two or more adjacent vertebral bodies are fused together. It is one of the most common approaches to alleviating various types of spinal pain, particularly pain associated with one or more affected intervertebral discs. While spine fusion generally helps to eliminate certain types of pain, it has been shown to decrease function by limiting the range of motion for patients in flexion, extension, rotation and lateral bending. Furthermore, the fusion creates increased stresses on adjacent non-fused motion segments and accelerated degeneration of the motion segments. Additionally, pseudarthrosis (resulting from an incomplete or ineffective fusion) may not provide the expected pain-relief for the patient. Also, the device(s) used for fusion, whether artificial or biological, may migrate out of the fusion site creating significant new problems for the patient.

Various technologies and approaches have been developed to treat spinal pain without fusion in order to maintain or recreate the natural biomechanics of the spine. To this end, significant efforts are being made in the use of implantable artificial intervertebral discs. Artificial discs are intended to restore articulation between vertebral bodies so as to recreate the full range of motion normally allowed by the elastic properties of the natural disc. Unfortunately, the currently available artificial discs do not adequately address all of the mechanics of motion for the spinal column.

It has been found that the facet joints can also be a significant source of spinal disorders and debilitating pain. For example, a patient may suffer from arthritic facet joints, severe facet joint tropism, otherwise deformed facet joints, facet joint injuries, etc. These disorders lead to spinal stenosis, degenerative spondylolithesis, and/or isthmic spondylotlisthesis, pinching the nerves which extend between the affected vertebrae.

Current interventions for the treatment of facet joint disorders have not been found to provide completely successful results. Facetectomy (removal of the facet joints) may provide some pain relief; but as the facet joints help to support axial, torsional, and shear loads that act on the spinal column in addition to providing a sliding articulation and mechanism for load transmission, their removal inhibits natural spinal function. Laminectomy (removal of the lamina, including the spinal arch and the spinous process) may also provide pain relief associated with facet joint disorders; however, the spine is made less stable and subject to hypermobility. Problems with the facet joints can also complicate treatments associated with other portions of the spine. In fact, contraindications for disc replacement include arthritic facet joints, absent facet joints, severe facet joint tropism, or otherwise deformed facet joints due to the inability of the artificial disc (when used with compromised or missing facet joints) to properly restore the natural biomechanics of the spinal motion segment.

While various attempts have been made at facet joint replacement, they have been inadequate. This is due to the fact that prosthetic facet joints preserve existing bony structures and therefore do not address pathologies which affect facet joints themselves. Certain facet joint prostheses, such as those disclosed in U.S. Pat. No. 6,132,464, are intended to be supported on the lamina or the posterior arch. As the lamina is a very complex and highly variable anatomical structure, it is very difficult to design a prosthesis that provides reproducible positioning against the lamina to correctly locate the prosthetic facet joints. In addition, when facet joint replacement involves complete removal and replacement of the natural facet joint, as disclosed in U.S. Pat. No. 6,579,319, the prosthesis is unlikely to endure the loads and cycling experienced by the vertebra. Thus, the facet joint replacement may be subject to long-term displacement. Furthermore, when facet joint disorders are accompanied by disease or trauma to other structures of a vertebra (such as the lamina, spinous process, and/or transverse processes) facet joint replacement is insufficient to treat the problem(s).

Most recently, surgical-based technologies, referred to as “dynamic posterior stabilization,” have been developed to address spinal pain resulting from more than one disorder, when more than one structure of the spine have been compromised. An objective of such technologies is to provide the support of fusion-based implants while maximizing the natural biomechanics of the spine. Dynamic posterior stabilization systems typically fall into one of two general categories: (1) interspinous spacers and (2) posterior pedicle screw-based systems.

Examples of interspinous spacers are disclosed in U.S. Pat. Nos. Re. 36,211, 5,645,599, 6,695,842, 6,716,245 and 6,761,720. The spacers, which are made of either a hard or compliant material, are placed between adjacent spinous processes. Because the interspinous spacers involve attachment to the spinous processes, use of these types of systems is limited to applications where the spinous processes are uncompromised and healthy.

Examples of pedicle screw-based systems are disclosed in U.S. Pat. Nos. 5,015,247, 5,484,437, 5,489,308, 5,609,636 and 5,658,337, 5,741,253, 6,080,155, 6,096,038, 6,264,656 and 6,270,498. These types of systems involve the use of screws which are positioned in the vertebral body through the pedicle. Certain types of these pedicle screw-based systems may be used to augment compromised facet joints, while others require removal of the spinous process and/or the facet joints for implantation. One such system, the Zimmer Spine Dynesys® employs a cord which is extended between the pedicle screws and a fairly rigid spacer which is passed over the cord and positioned between the screws. While this system is able to provide load sharing and restoration of disc height, because it is so rigid, it does not effectively preserve the natural motion of the spinal segment into which it is implanted. Other pedicle screw-based systems employ articulating joints between the pedicle screws.

There remains a need for minimally invasive methods and devices for bone stabilization procedures, including but not limited to spinal segment stabilization procedures such as dynamic spinal segment stabilization procedures. There is a need for procedures that are simple to perform and reliably achieve the desired safe and effective outcome. Goals of these new procedures and instruments include minimizing the size of the incision and reducing the amount of muscle dissection in order to shorten recovery times, improve procedure success rates and reduce the number of resultant adverse side effects.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A-B illustrate perspective views of a portion of the human spine having two vertebral segments, where the spinous process and the lamina of the superior vertebra have been resected in FIG. 1B.

FIGS. 2A-C illustrate left side, dorsal and top views, respectively, of the spinal segments of FIG. 1A under going various motions.

FIGS. 3A-C illustrate a side sectional view of a bone stabilization device, consistent with the present invention, placed between a first bone location and a second bone location and shown in various levels of rotation of a pivoting arm of the hinged assembly of the device.

FIG. 4 illustrates a perspective view of a bone stabilization device consistent with the present invention.

FIGS. 4A and 4B illustrate a perspective view of the bone stabilization device of FIG. 4 shown with the pivoting arm rotating through an arc and engaged with an attaching cradle, respectively.

FIG. 5 illustrates an exploded perspective view of a bone stabilization device consistent with the present invention.

FIGS. 6A-H illustrate multiple side sectional views of a method of placing a bone stabilization device in a minimally invasive percutaneous procedure, consistent with the present invention.

FIG. 7 illustrates a perspective view of a slotted cannula consistent with the present invention.

FIG. 7A illustrates a perspective view of the slotted cannula of FIG. 7 positioned to access or place a device at a vertebral segment of a patient.

FIG. 8 illustrates a perspective view of a pivoting tool consistent with the present invention.

FIG. 8A illustrates a perspective view of the pivoting tool of FIG. 8 positioned to rotate a pivoting arm of a hinged assembly of the present invention.

FIG. 9 illustrates a side schematic view of a hinged assembly consistent with the present invention wherein the pivoting arm includes a functional element along its length.

FIGS. 9A-B illustrate perspective views of hinged assemblies of the present invention in which a functional element includes a dynamic motion element, a tension-compression spring and a coiled spring respectively.

FIG. 9C illustrates a side sectional view of the bone stabilization device of the present invention with the hinged assembly of FIG. 9B shown in multiple stages of rotating its pivoting arm.

FIGS. 10A-C show side sectional views of a stabilization method consistent with the present invention in which multiple vertebral segments are stabilized.

FIGS. 11A-B illustrate perspective views of pairs of pivoting arms consistent with the present invention, shown with “stacked” and “side-by-side” configurations, respectively, for poly-segment (more than two segment) bone stabilization.

FIGS. 12A-B illustrate perspective views of pairs of pivoting arms consistent with the present invention, shown with “stacked” and “side-by-side” configurations, respectively, for poly-segment bone stabilization, wherein each pivoting arm includes an integral coiled spring.

FIG. 13 illustrates a side sectional view of a poly-segment bone stabilization system consistent with the present invention, in which the pivoting arm pair of FIG. 12A or 12B has been secured to vertebrae and engaged at their midpoint with a receiving assembly, also secured to a vertebra.

FIGS. 14A-C illustrate hinged assemblies consistent with the present invention including, respectively, a pivoting arm with “snap-in” axle, a pivoting arm with a captured axle, and a pivoting arm with a flexible segment.

FIGS. 15A-B illustrates perspective views of bone stabilization devices consistent with the present invention wherein additional set screws are placed to secure the pivoting arm.

FIG. 16 illustrates a side sectional view of a method consistent with the present invention in which an already placed bone stabilization device is accessed for adjustment, removal or partial removal.

FIG. 17 illustrates a side sectional view of a bone stabilization device consistent with the present invention in which each bone anchor includes a removable and/or replaceable threaded core and the pivoting arm includes a functional element.

FIG. 18 illustrates a side view of a bone stabilization device consistent with the present invention in which the pivoting arm comprises a telescoping assembly such that the radius of the arc during rotation of the pivoting arm is greatly reduced.

FIG. 19 illustrates a top view of a hinged assembly consistent with the present invention in which the hinged assembly comprises multiple pivoting arms.

FIG. 19A illustrates a side sectional view of a bone stabilization device of the present invention in which the hinged assembly of FIG. 19 is anchored to a bone segment, and the first pivoting arm rotates to a first receiving assembly and the second pivoting arm rotates to a second receiving assembly.

FIG. 20 illustrates an end view of receiving assembly consistent with the present invention in which the cradle includes a projection that is configured to capture a pivoting arm.

FIGS. 20A-B illustrate side and end views, respectively, of a bone stabilization device consistent with the present invention using the receiving assembly of FIG. 20 and shown with the pivoting arm captured by the cradle of the receiving assembly.

FIG. 21 illustrates a side sectional view of a hinged assembly consistent with the present invention in which two mechanical advantage elements are integral to the hinged assembly.

FIGS. 22A-B illustrate side sectional and top views of a bone stabilization device of the present invention in which two hinged assemblies are secured to bone in an adjacent, connecting configuration with a receiving assembly secured at one end.

FIG. 23 illustrates a perspective view of a bone stabilization device according to an embodiment of the present invention in which a mechanism is provided for driving the screw despite the presence of the rod.

FIG. 24 illustrates an exploded view of the device of FIG. 23.

FIG. 25 illustrates a side sectional view of the device of FIG. 23.

FIG. 26 illustrates a top view of the device of FIG. 23.

FIGS. 27A-B show a clam-shell capture mechanism for a pivoting rod to attach to a bone anchor.

FIGS. 28A-B show a screw-thread capture mechanism for a pivoting rod to attach to a bone anchor.

FIGS. 29A-B show top and side views of a frictional-fit engagement for a pivoting rod to attach to a seat of a bone anchor.

FIGS. 30A-B show top and side views of an alternative embodiment of a frictional-fit engagement for a pivoting rod to attach to a seat of a bone anchor.

FIGS. 31A-D show assemblies for frictional-fit engagements for a pivoting rod to attach to a seat of a bone anchor, where the degree of range of motion is controllably adjusted.

FIGS. 32A-C show assemblies for frictional-fit engagements for a pivoting rod to attach to a seat of a bone anchor.

FIGS. 33A-B show an alternative embodiment of a rod and bone anchor assembly.

FIG. 34 shows a device that may be employed in an embodiment of a rod and bone anchor assembly.

FIGS. 35A-C show a system for automatic distraction or compression.

FIGS. 36A-B show an embodiment related to that of FIGS. 49A-C in which one ball end of a pivoting rod is movable.

FIG. 37 shows a top view of a rod and seat system in which screws are used to widen a slot, frictionally securing the rod to the seat.

FIGS. 38A-C show a dual-pivoting rod assembly for use in multi-level bone stabilization or fixation.

FIGS. 39A-D show details of an embodiment related to that of FIG. 41A-C.

FIGS. 40A-C show a dual arm system with a unitary hinged assembly employing adjustable-length rods.

FIGS. 41A-F show a dual arm system with a unitary hinged assembly employing multiple axles for the pivoting rods.

FIGS. 42A-D show an alternative dual arm system with a unitary hinged assembly employing multiple axles for the pivoting rods.

FIGS. 43A-C show a dual arm system with a unitary hinged assembly employing pivoting rods with an offset angle.

FIGS. 44A-E show a dual arm system with a unitary hinged assembly employing pivoting rods, each with a complementary taper.

FIGS. 45A-B shows top and side views of a bone screw system employing a partial skin incision to allow use of a long pivoting rod.

FIGS. 46A-B show side views of a bone screw system employing a pivoting rod with a sharpened edge to assist in skin dissection.

FIG. 47 shows a side view of a bone screw system employing a pivoting rod with a resiliently-biased feature.

FIG. 48 shows a side view of a bone screw system employing a pivoting rod with a curved feature.

FIG. 49 shows a side view of a bone screw system employing a receiving assembly configured such as to provide confirmation of attachment of the pivoting rod.

FIGS. 50A-D show views of a bone screw system employing radiopaque markers to confirm placement and pivoting rod rotation.

FIGS. 51A-B show views of a bone screw system employing a hinged pivoting rod.

FIGS. 52A-B show a bone screw system with a guidewire lumen through the pivoting rod and bone anchor.

FIG. 53 shows a view of a bone screw system with a custom cannula to accommodate a dynamic stabilization element or other custom functional element.

FIG. 54 shows a target needle that is used to penetrate through the skin up to and through the pedicle.

FIGS. 55A-D show various embodiments of a guidewire that is used for over-the-wire insertion and exchange of various cannulated devices.

FIGS. 56A-E show one of a series of cannulated dilators that may be used to sequentially dilate and expand the tissue between the entry site established by the target needle and the pedicle.

FIG. 57 shows an alternative embodiment of the dilator that includes advancable grippers such as retractable teeth on their distal ends.

FIG. 58 shows an alternative embodiment of the dilator that includes helical grooves.

FIG. 59 shows an expandable or tapered dilator.

FIG. 60A shows a tap device that is used to tap a hole in the bone in which the screw will be implanted; FIGS. 60B-C shows the handle of the tap device with an integrated optical motion sensor and a visual display.

FIGS. 61A-E show a screw tower assembly (STA) tool that is used to insert the pedicle screw assembly.

FIG. 62 shows a locking tool having a tubular body that includes engaging lugs on its distal end.

FIGS. 63A-F show alternative embodiments of a polyaxial screwdriver that includes a handle and a tubular body to which the handle attaches.

FIGS. 64A-F and 65A-D show various perspective views of a primary alignment guide that is employed to align the seat of the screw assembly.

FIG. 66 shows the distal end of the primary alignment guide fitting over the proximal end of the STA 1130.

FIGS. 67A-I show various perspective views of a secondary alignment guide that forms a hinge or pivot with the primary alignment guide.

FIG. 68 shows a rod length measuring tool that is used to determine the appropriate rod length that is needed.

FIGS. 69A-F show a tissue splitter that is used to dissect the tissue between the seats of the screws so that a subcutaneous path is created for the rod to rotate.

FIG. 70 shows a rod introducer assembly that is used to implant the rod after the screw assemblies have been inserted.

FIGS. 71A-D shows a rod pusher 1194 to pivot the rod 903 so that it engages with both screw assemblies.

FIGS. 72A-F shows a cap inserter instrument that is used to place the cap assembly into the grooves of the seat to secure the end of the rod.

FIG. 73 shows a cap reducer that may be used to facilitate advancement of the cap assembly in the threads of the cap seat.

FIGS. 74A-H show a distraction/compression instrument that is used to either distract or compress the vertebra to which the bone stabilization device is attached.

FIGS. 75A-D show the distraction/compression instrument attached at a location above and below, respectively, the pivot point formed by the primary and secondary alignment guides.

FIG. 76 shows a torque indicating driver that is used to tighten the setscrew in the cap assembly.

FIG. 77 shows a torque stabilizer attached to one of the alignment guides so that the operator can stabilize the system during the final tightening procedure.

FIGS. 78A-B show a guidewire clip that may be used to prevent the guidewire from inadvertently advancing during the procedure.

FIG. 79 shows a rod holder that may be inserted through the cannula of the rod introducer assembly shown in FIG. 70 to hold the rod in place.

FIGS. 80A-C show a cap release tool that may be used to facilitate the removal of the cap inserter instrument.

FIG. 81A shows an exploded view of one embodiment of the bone stabilization device, which will be used to illustrate the system of tools that may be used to properly place the device in a minimally invasive percutaneous procedure; FIG. 81B shows the screw assembly and FIG. 81C shows the cap assembly.

FIGS. 82A-F shows an alternative embodiment of the tissue splitter in which the blade cuts through tissue by pushing on the handle rather than pulling.

FIG. 83 shows the target needle as it gains access to the pedicle.

FIG. 84 shows the target needle being removed while leaving the guide in place.

FIG. 85 shows the guidewire being inserted through the guide.

FIG. 86 shows an over-the-wire “exhange” in which the guide is removed, leaving the guidewire in place.

FIG. 87 shows the first of a series of dilators being placed over-the-wire.

FIG. 88 shows a second dilator being placed over the first dilator.

FIG. 89 shows a third dilator being placed over the second dilator.

FIG. 90 shows the torque stabilizer being used to exert force on the dilator.

FIG. 91 shows the largest diameter dilator after the smaller dilators have been removed.

FIG. 92 shows the tap device being assembled.

FIG. 93 shows the tap device being placed over-the-wire and through the largest diameter dilator.

FIG. 94 shows the guidewire clip attached to the guidewire to maintain the guidewire's position.

FIG. 95 shows the tapped hole that is created by the tap device.

FIGS. 96A-B show the STA being attached to the screw assembly.

FIGS. 97A-B show the locking tool being connected to the STA.

FIGS. 98A-B show the screw assembly after being locked to the STA.

FIG. 99 shows the screw assembly is engaged with the STA after the locking tool is removed.

FIGS. 100A-B shows the polyaxial screwdriver being assembled.

FIGS. 101A-C shows the polyaxial screwdriver being attached to STA.

FIGS. 102A-D show the assembly, STA and screwdriver being inserted over the wire into the pedicle.

FIGS. 103A-B show the first and second STAs after the screwdriver is removed.

FIGS. 104A-C show the primary alignment guide (PAG) being placed over the first STA.

FIGS. 105A-D show the secondary alignment guide (SAG) being placed over the second STA.

FIGS. 106A-B show the locking tool being attached to the SAG after the cross pin of the SAG and the hook of the PAG have been engaged to create a hinge.

FIGS. 107A-C show the rod gauge indicator being attached to the secondary alignment guide and the rod gauge measurement device being attached to the primary alignment guide.

FIGS. 108A-B show the tissue splitter being inserted into the SAG.

FIGS. 109A-D show the rod being inserted into the SAG.

FIGS. 110A-E show the rod pusher being used to pivot the rod into position.

FIGS. 111A-D show the cap inserter instrument being attached to the cap assembly, and FIGS. 112A-C show the cap inserter instrument being secured to the primary alignment guide.

FIGS. 113A-B show the first and second cap inserter instruments secured in the PAG and the SAG, respectively.

FIG. 114 shows both bone stabilization devices after being installed in the vertebra.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

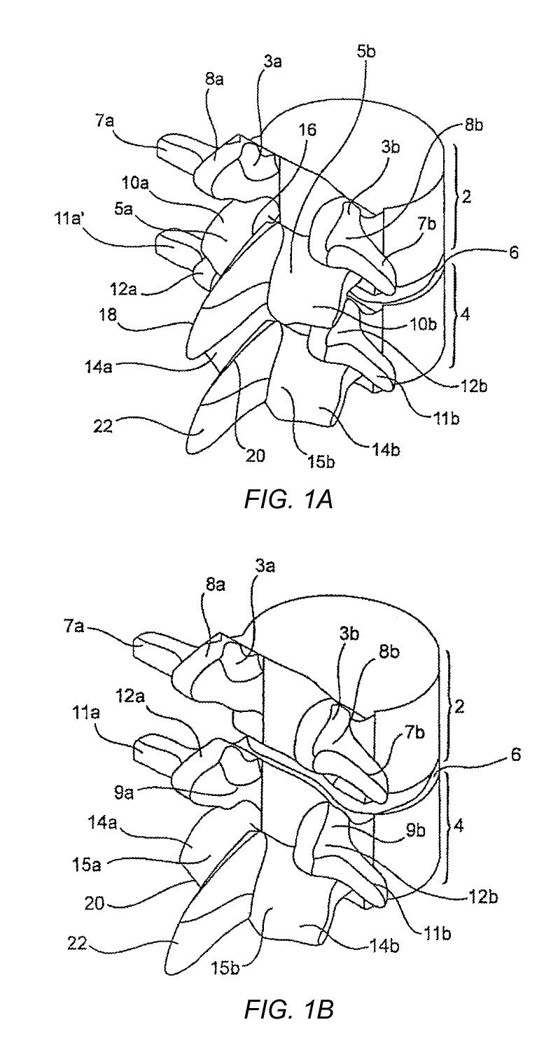

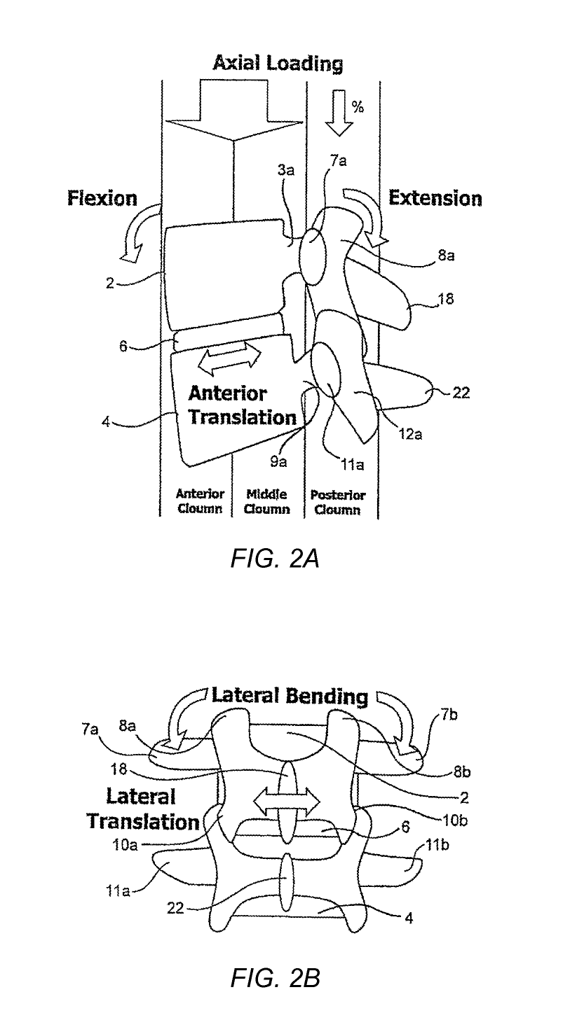

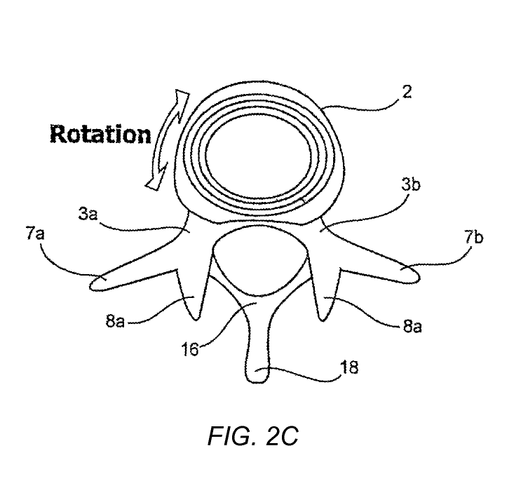

The present invention will now be described in greater detail by way of the following description of exemplary embodiments and variations of the systems and methods of the present invention. While more fully described in the context of the description of the subject methods of implanting the subject systems, it should be initially noted that in certain applications where the natural facet joints are compromised, as illustrated in FIG. 1B, inferior facets 10a and 10b, lamina 5a and 5b, posterior arch 16 and spinous process 18 of superior vertebra 2 of FIG. 1A may be resected for purposes of implantation of certain of the dynamic stabilization systems of the present invention. In other applications, where possible, the natural facet joints, lamina and/or spinous processes are spared and left intact for implantation of other dynamic stabilization systems of the present invention.

It should also be understood that the term “system”, when referring to a system of the present invention, most typically refers to a set of components which includes multiple bone stabilization components such as a superior or cephalad (towards the head) component configured for implantation into a superior vertebra of a vertebral motion segment and an inferior or caudal (towards the feet) component configured for implantation into an inferior vertebra of a vertebral motion segment. A pair of such component sets may include one set of components configured for implantation into and stabilization of the left side of a vertebral segment and another set configured for the implantation into and stabilization of the right side of a vertebral segment. Where multiple bone segments such as spinal segments or units are being treated, the term “system” may refer to two or more pairs of component sets, i.e., two or more left sets and/or two or more right sets of components. Such a multilevel system involves stacking of component sets in which each set includes a superior component, an inferior component, and one or more medial components therebetween.

The superior and inferior components (and any medial components therebetween), when operatively implanted, may be engaged or interface with each other in a manner that enables the treated spinal motion segment to mimic the function and movement of a healthy segment, or may simply fuse the segments such as to eliminate pain and/or promote or enhance healing. The interconnecting or interface means include one or more structures or members that enables, limits and/or otherwise selectively controls spinal or other body motion. The structures may perform such functions by exerting various forces on the system components, and thus on the target vertebrae. The manner of coupling, interfacing, engagement or interconnection between the subject system components may involve compression, distraction, rotation or torsion, or a combination thereof. In certain embodiments, the extent or degree of these forces or motions between the components may be intraoperatively selected and/or adjusted to address the condition being treated, to accommodate the particular spinal anatomy into which the system is implanted, and to achieve the desired therapeutic result.

In certain embodiments, the multiple components, such as superior and inferior spinal components, are mechanically coupled to each other by one or more interconnecting or interfacing means. In other embodiments, components interface in an engaging manner, which does not necessary mechanically couple or fix the components together, but rather constrains their relative movement and enables the treated segment to mimic the function and movement of a healthy segment. Typically, spinal interconnecting means is a dorsally positioned component, i.e., positioned posteriorly of the superior and inferior components, or may be a laterally positioned component, i.e., positioned to the outer side of the posterior and inferior components. The structures may involve one or more struts and/or joints that provide for stabilized spinal motion. The various system embodiments may further include a band, interchangeably referred to as a ligament, which provides a tensioned relationship between the superior and inferior components and helps to maintain the proper relationship between the components.

Reference will now be made in detail to the present embodiments of the invention, examples of which are illustrated in the accompanying drawings. Wherever possible, the same reference numbers will be used throughout the drawings to refer to the same or like parts.

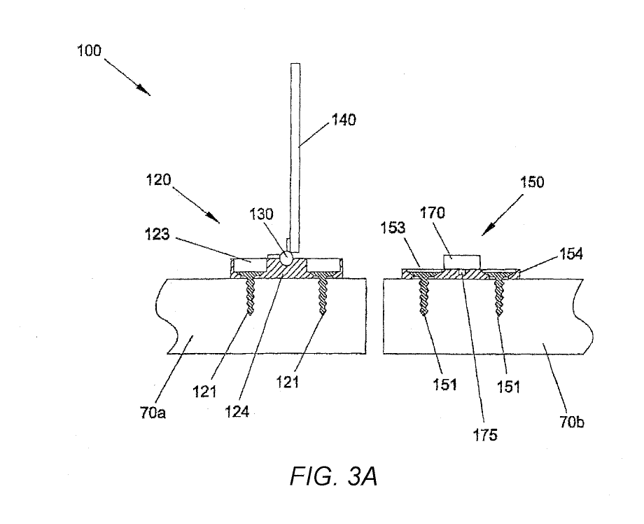

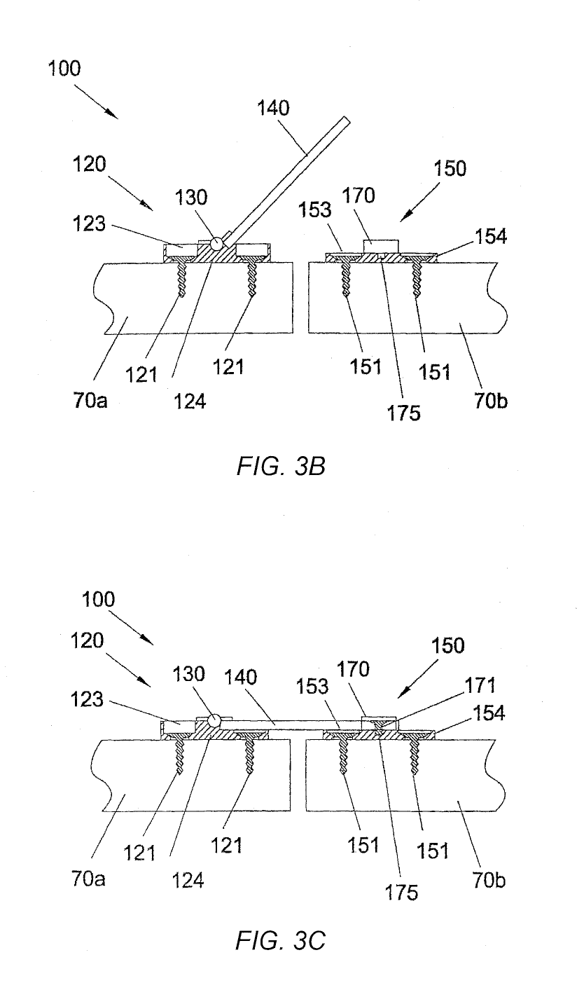

Referring now to FIGS. 3A-3C, there is illustrated a bone stabilization device 100 operatively implanted into a patient. Device 100 includes hinged assembly 120 which has been attached to first bone segment 70a, and a receiving assembly 150 which has been attached to second bone segment 70b. Bone segments 70a and 70b can take on numerous forms, such as two segments from a broken bone such as a femur, tibia and/or fibula of the leg, or the humerus, radius and/or ulna bones of the forearm. In a preferred embodiment, bone segments 70a and 70b are vertebrae of the patient, such as adjacent vertebra or two vertebra in relative proximity to each other. Device 100 may be implanted to promote healing, reduce or prevent pain, restore motion, provide support and/or perform other functions. Device 100 may be utilized to stabilize bone segments, to prevent or limit movement and/or to dynamically control movement such as to provide restoring or cushioning forces. Device 100, specifically applicable to uses wherein the bone segments 70a and 70b are vertebrae of the patient, may stabilize these segments yet dynamically allow translation, rotation and/or bending of these spinal segments, such as to restore an injured or diseased spinal segment to a near-healthy state. In an alternative embodiment, device 100 is inserted into a patient, such as a healthy or unhealthy patient, to enhance spinal motion, such as to increase a healthy patient's normal ability to support large amounts of weight, such as for specific military applications, and/or be conditioned to work in unusual environments such as the gravity reduced environments of locations outside earth's atmosphere or at high pressure locations such as in deep-water scuba diving.

Device 100 may be implanted for a chronic period, such as a period over thirty days and typically an indefinite number of years, a sub-chronic period such as a period greater than twenty-four hours but less than thirty days, or for an acute period less than 24 hours such as when device 100 is both placed and removed during a single diagnostic or therapeutic procedure. Device 100 may be fully implanted under the skin of the patient, such as when chronically implanted, or may exist both outside the skin and in the patient's body, such as applications where the stabilization components reside above the patient's skin and anchoring screws pass through the skin and attach these stabilization components to the appropriate bone structures.

Referring back to FIGS. 3A through 3C, hinged assembly 120 is anchored to bone segment 70a with two screws 121, such as bone screws or pedicle screws when bone segment 70a is a vertebra, passing through base 124. Screws 121 may be inserted in a pre-drilled hole, such as a hole drilled over a pre-placed guidewire with a cannulated bone drill and/or the screws may include special tips and threads that allow the screws to self-tap their insertion. The screws may include one or more treatments or coatings, such as including a Teflon layer that supports long-term removal of the screw from the bone, such as to replace an implanted component. In a preferred embodiment, screw 121 includes threads that include a surface configured to prevent anti-rotation or loosening, such as an adhesive surface or a grooved surface whose grooves are aligned to support rotation in a single direction only. In another preferred embodiment, the screws include expansion means, such as hydraulic or pneumatic expansion means, which allow the diameter of the thread assembly to slightly increase or decrease on demand to facilitate secure long-term attachment, as well as ease of removal. Base 124 includes recess 123, which is a countersink that allows the tops of screws 121 to reside below the top surface of base 124 when anchored to bone segment 70a.

In an alternative embodiment, an articulating element, not shown, is included allowing hinged assembly 120 to move relative to bone segment 70a. Attached to base 124 is hinge 130, which rotatably attaches base 124 to pivoting arm 140. Hinge 130 shown is a pin and bushing construction similar to a door hinge. Numerous alternatives may be employed, additionally or alternatively, some of which are described in detail in reference to subsequent figures, without departing from the spirit can scope of this application. Hinge 130 may include a ball and socket construction, or may simply consist of a flexible portion integral to pivoting arm 140, base 124 and/or a flexible element that couples base 124 to pivoting arm 140. Hinge 130 may be configured to allow one or more degrees of freedom of motion of pivoting arm 140 relative to base 124. Hinge 130 may be an attachable hinge, such as a hinge that is assembled by an operator during the surgical procedure but prior to passing hinged assembly 120 through the skin of the patient. Alternatively hinge 130 may be preattached, and may not be able to be disassembled by the operator during or subsequent to the implantation procedure.

Also depicted in FIGS. 3A through 3C is receiving assembly 150, which is configured to be securely attached to second bone segment 70b with attachment screws 151, which are preferably similar to attachment screws 121. Screws 151 are similarly passed through base 154 such that the head of screw 151 resides entirely within recess 153. In an alternative embodiment, an articulating element, not shown, is included allowing receiving assembly 150 to move relative to bone segment 70b. Securedly attached to base 154 is cradle 170, configured to attach to the distal end of pivoting arm 140. Cradle 170 may be fixedly attached to base 154, or may include an articulating member, not shown, to allow a limited range of motion between cradle 170 and base 154. Cradle 170 includes threads 175 which are configured to receive a securing element, such as a set screw, to maintain pivoting arm 140 in a secured connection with receiving assembly 150.

Referring specifically to FIG. 3B, pivoting arm 140 has been rotated approximately forty-five degrees in a clockwise direction, such that the distal end of arm 140 has traversed an arc in the direction toward cradle 170. Referring specifically to FIG. 3C, arm 140 has been rotated approximately an additional forty-five degrees, a total of ninety degrees from the orientation shown in FIG. 3A, such that the distal end of arm 140 is in contact or otherwise in close proximity with cradle 170. A securing device, locking screw 171 has been passed through a hole in the distal end of arm 140 and threaded into threads 175 of cradle 170, such that a stabilizing condition has been created between first bone segment 70a and second bone segment 70b. This stabilizing condition, as has been described above, can take on a number of different forms, singly or in combination, such as fixed stabilization and dynamic stabilization forms. Dynamic stabilization provides the benefit of allowing motion to occur, such as normal back or other joint motions that a fixed stabilization device may prevent or compromise.

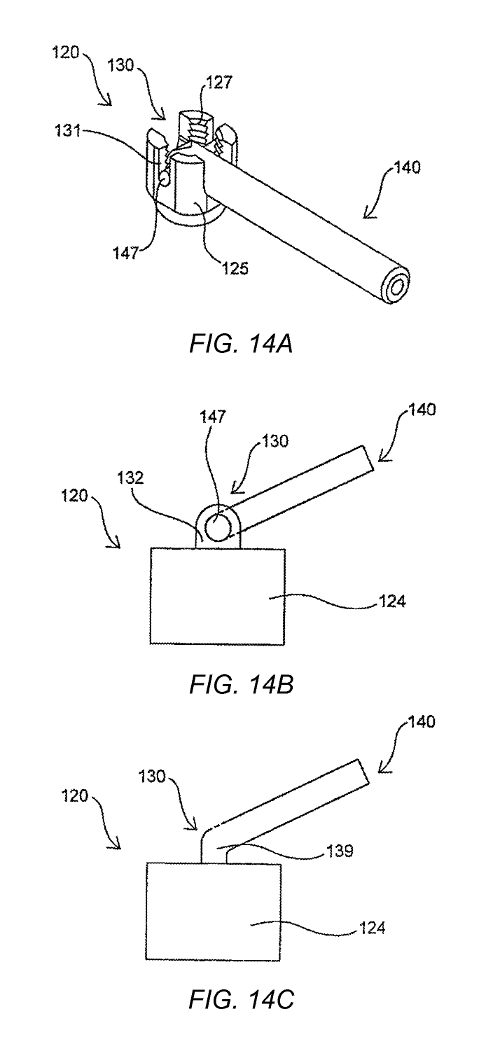

Cradle 170 of FIGS. 3A through 3C includes a “U′ or “V” shaped groove, end view not shown, which acts as a guide and accepts the distal end of arm 140. Arm 140 is securedly attached in a fixed connection shown through the placement of screw 171 through arm 140 and in an engaged position with threads 175 of cradle 170. In an alternative embodiment, dynamic stabilization between first bone segment 70a and second bone segment 70b is achieved by the creation of a dynamic or “movable” secured connection between the distal end of arm 140 and cradle 170. In an alternative or additional embodiment, dynamic stabilization between first bone segment 70a and second bone segment 70b is achieved via a dynamic secured connection between hinge 130 and base 124 of hinged assembly 120. In yet another additional or alternative embodiment, dynamic stabilization of first bone segment 70a and second bone segment 70b is achieved via pivoting arm 140, such as an arm with a spring portion, such as a coil or torsional-compress spring portion, or by an otherwise flexible segment integral to arm 140. Arm 140 may take on numerous forms, and may include one or more functional elements, described in detail in reference to subsequent figures. Arm 140 may include multiple arms, such as arms configured to perform different functions. In an alternative embodiment, described in detail in reference to FIG. 14C, arm 140 may include a hinge-like flexible portion, performing the function of and obviating the need for hinge 130.

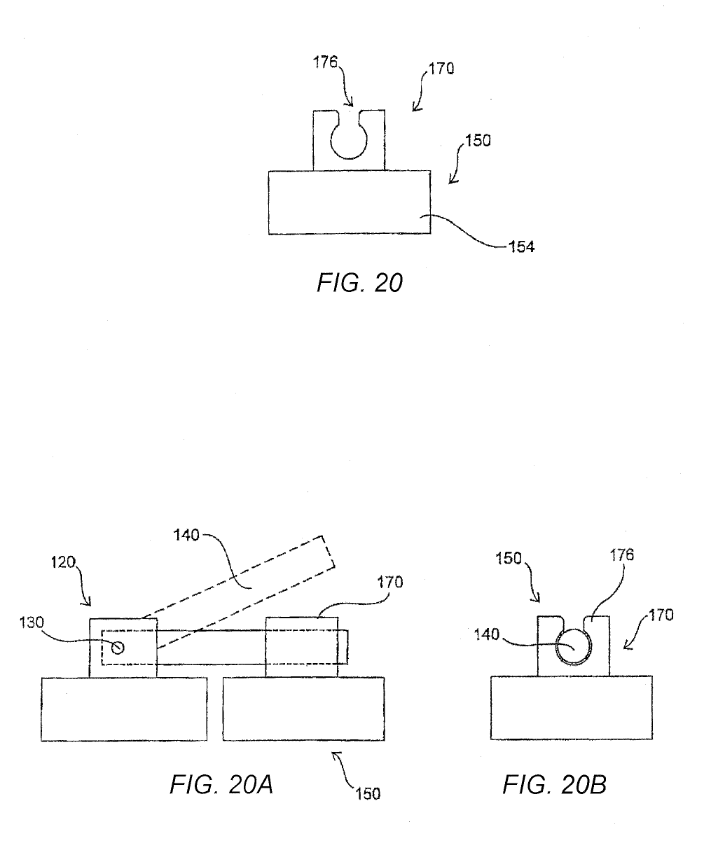

Cradle 170 may also take on numerous forms, in addition or alternative to the grooved construction of FIGS. 3A through 3C. Cradle 170 performs the function of securing arm 140 to receiving assembly 150, such as via screw 171 engaging threads 175. In alternative embodiments, numerous forms of attaching a rod to a plate may be used, with or without a guiding groove, including retaining rings and pins, belts such as flexible or compressible belts, and other fixed or dynamic stabilization means. Screw 171 is placed by an operator, such as a clinician inserting and rotating screw 171 through a dilating cannula used in a minimally invasive percutaneous procedure, such that when screw 171 engages threads 175, pivoting arm 170 stabilizes hinged assembly 120 and receiving assembly 150 relative to each other, thus stabilizing first bone segment 70a and second bone segment 70b relative to each other. Insertion and engagement of screw 171 into threads 175 provides stabilization of hinged assembly 120 and receiving assembly 150 in two ways. First, motion between arm 140 and receiving assembly 150 is stabilized. Also, motion between arm 140 and base 124 of hinged assembly 120 is stabilized. In an alternative or additional embodiment, when pivoting arm 120 is pivoted, such as to the location shown in FIG. 3C, an automatic locking tab, not shown, is automatically engaged with further operation of the operator, such that pivoting arm 140 is prevented from pivoting back (in a counterclockwise direction as depicted in FIG. 3C). In another alternative or additional embodiment, described in detail in reference to FIGS. 20, 20A and 20B, an automatic engaging assembly is integral to cradle 170, such as a “U” shaped groove with a projection at the top of the “U” that allows arm 140 to snap in place into a secured configuration. Numerous other automatic or semi-automatic engaging mechanisms, such as those that limit rotation of arm 140 and/or secure the distal end of arm 140, may be employed in hinged assembly 120 and/or receiving assembly 150.

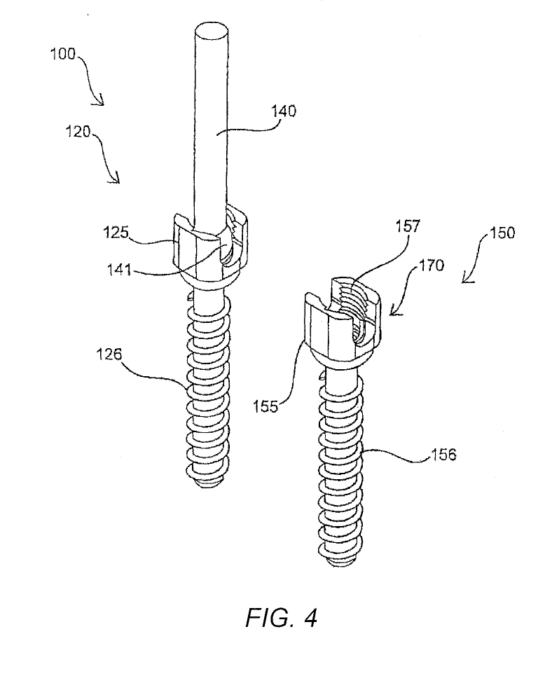

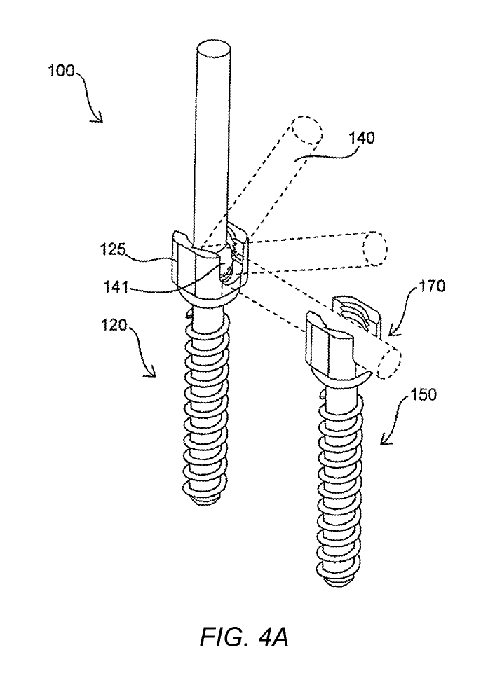

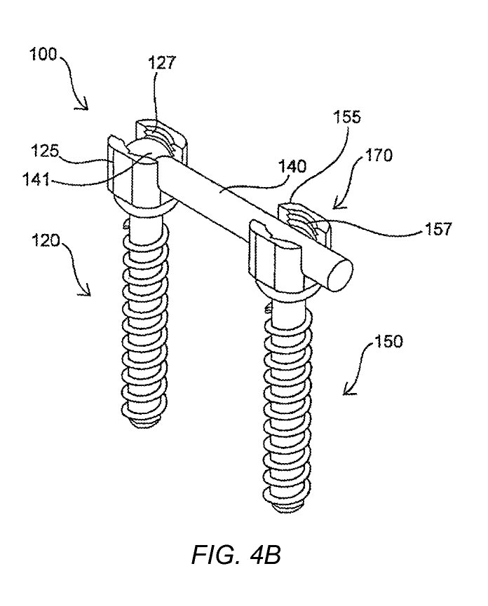

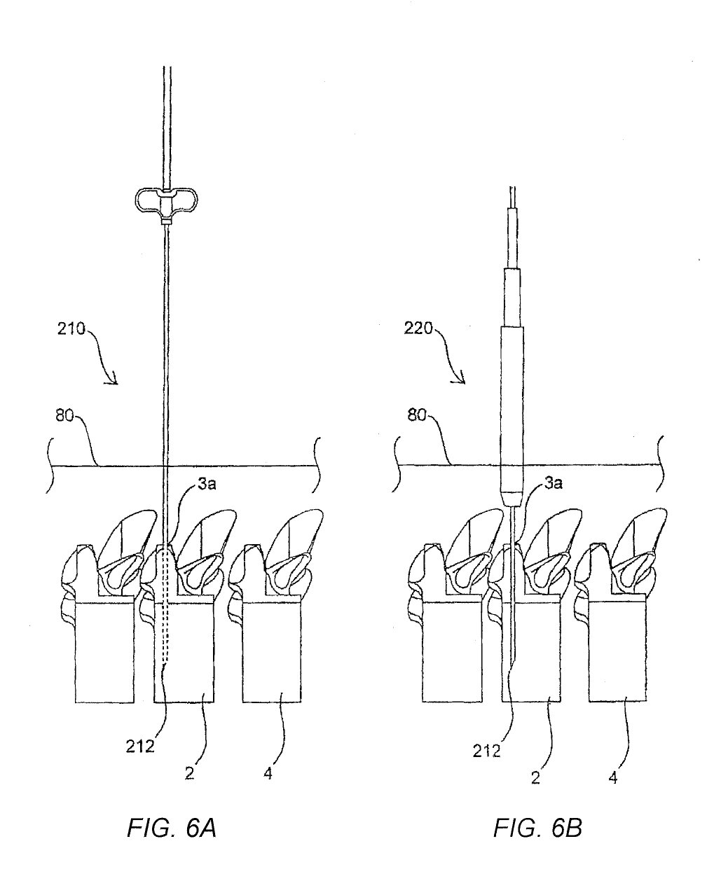

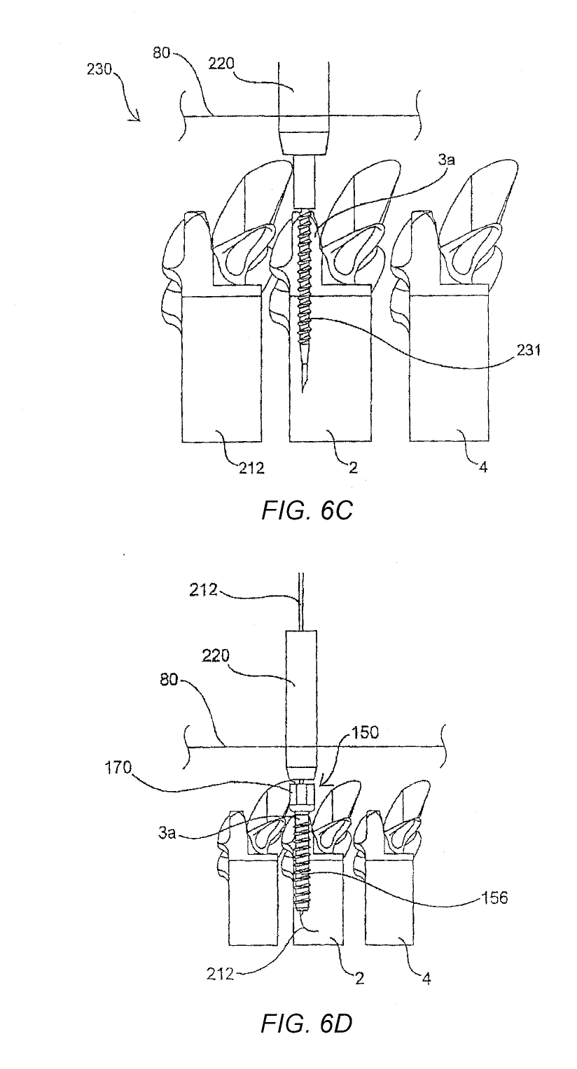

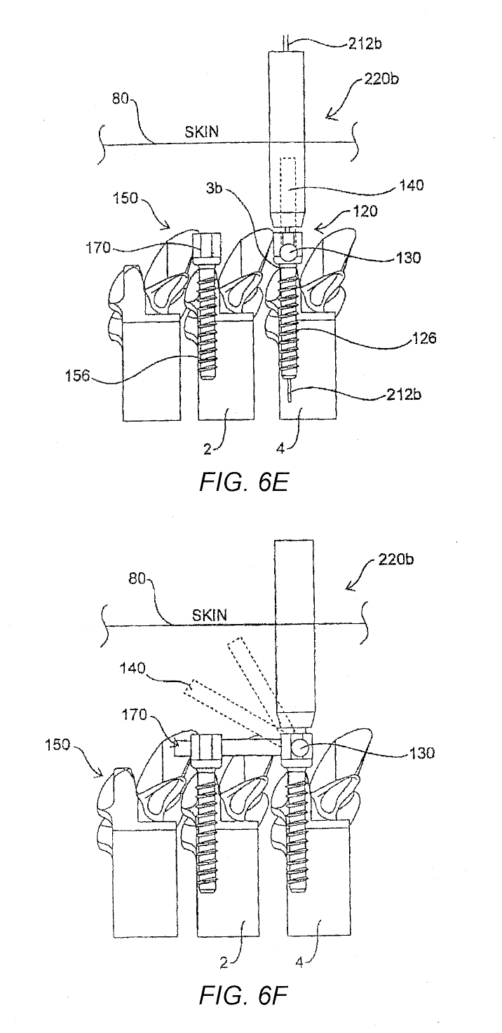

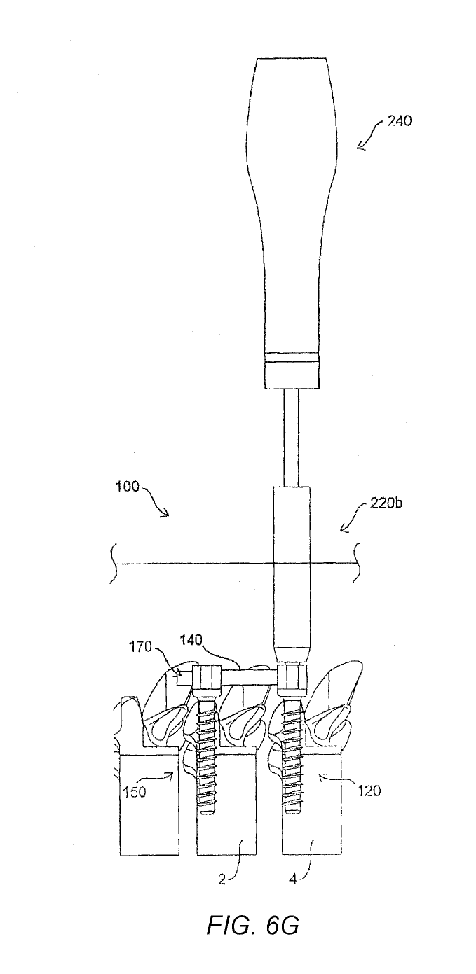

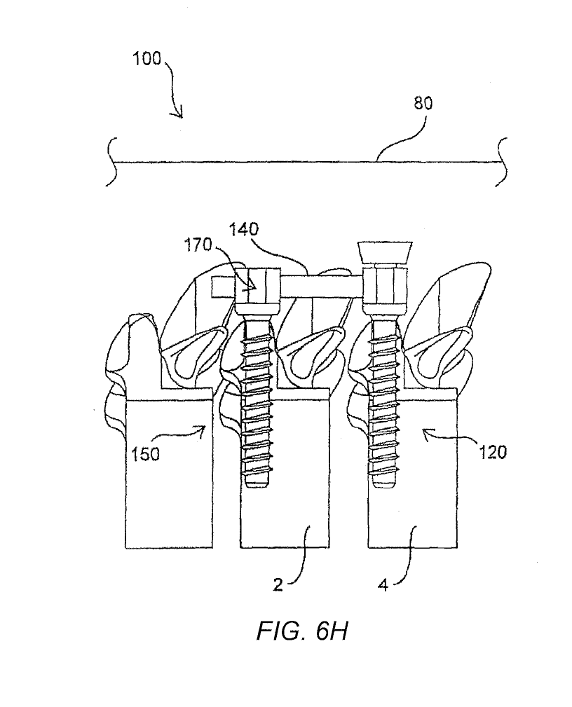

The components of system 100 of FIG. 3A are configured to be used in an open surgical procedure as well as a preferred minimally invasive procedure, such as an over-the-wire percutaneous procedure. Hinged assembly 120 and receiving assembly 150 preferably can each be inserted through one or more cannulae previously inserted through relatively small incisions through the patient's skin Devices and methods described in reference to FIGS. 4A, 4B and 4C, as well as FIGS. 6A through 6H include components with cannulated (including a guidewire lumen) bone anchors and other components with lumens and or slots that allow placement over a guidewire as well as one actions that can be completed with a guidewire in place, such actions including but not limited to: securing to bone, rotation of the pivoting arm, and securing of the pivoting arm to the receiving assembly.

Referring now to FIGS. 4, 4A and 4B, a preferred embodiment of a bone stabilization device of the present invention is illustrated in which each of the hinged assembly and the receiving assembly include cannulated bone screws that are configured to anchor into bone as rotated (while placed over a guidewire), and the hinged assembly pivoting arm hinge comprises a ball and socket configuration. Device 100 includes hinged assembly 120 comprising pivoting arm 140 and a bone anchoring portion including screw head 125 and bone threads 126. Screw head 125 includes one or more surfaces configured to engage with a tool, such as a percutaneously inserted socket wrench or screwdriver, to engage and rotate hinged assembly 120. Screw head 125, and all the similar screws of the present invention, are preferable polyaxial screw heads, such as the heads included in polyaxial pedicle screws commonly used in spine surgery. A lumen, not shown, passes through arm 140 and inside the tube surrounded by threads 126 such that hinged assembly 120, in the orientation shown in FIG. 4, can be placed into the patient through a cannula and over a previously placed guidewire, such as a “K-wire” commonly used in bone and joint procedures.

At the end of arm 140 is ball end 141, which is rotationally received and captured by screw head 125. Arm 140 can be inserted into screw head 125 by an operator, or may be provided in a pre-attached state. Arm 140 can be removable from screw head 125, or may be permanently, though rotatably, attached, whether provided in a “to-be-assembled” or a pre-assembled state. The ball and socket design of FIG. 4 allows multi-directional rotation of pivoting arm 140. Alternative designs, may allow a single degree of freedom, and/or may allow more sophisticated trajectories of travel for the distal end of arm 140.

System 100 further includes receiving assembly 150, which similarly includes a bone anchor comprising screw head 155, preferably a polyaxial screw head, and bone threads 156. Within the tube surrounded by bone threads 156 is a guidewire lumen that is configured to allow carrier assembly 150 to be placed through a cannula and over a guidewire that has previously been placed into the bone of a patient. Screw head 155 includes one or more surfaces configured to engage with a tool, such as a percutaneously inserted socket wrench or screwdriver, to engage and rotate receiving assembly 150. Cradle 170 comprises a “U” shaped groove that is sized and configured to accept and capture the distal end of pivoting arm 140. Cradle 170 may include positive engagement means such as threads 157, or other securing means such as a projecting member that is configured to provide a snap fit, magnetic holding means, pivoting engagement means such as a rotatable holding arm, adhesive holding means, or other retention elements all not shown.

Referring specifically to FIG. 4A, pivoting arm 140 is shown in multiple stages of rotation, including the starting position of FIG. 4 in which pivoting arm 140 and threads 126 are linearly aligned to allow over-the-wire insertion. After threads 126 are properly engaged with bone, pivoting arm 140 is rotated, in a clockwise direction as shown, to a point in which it engages with receiving assembly 150, preferably a near ninety degree rotation as shown, but alternatively a smaller or greater angle as determined by the orientation of the two bone segments to be stabilized. Arm 140 may be rotated with the guidewire removed, or may include a slot, not shown, that allows arm 140 to “separate” from the guidewire as arm 140 is rotated. In an alternative embodiment, hinged assembly 120 includes a cannulated screw, but arm 140 is not cannulated, traveling along side the guidewire during insertion, and rotating about the guidewire during rotation and bone anchoring of threads 126. In this alternative embodiment, a slot is not required to rotate arm 140, in a direction away from central axis of the in-place guidewire.

Referring now specifically to FIG. 4B, pivoting arm 140 has been rotated and engaged with cradle 170 of receiving assembly 150. In the preferred method of placing system 100 components through cannulae and over previously placed guidewires, pivoting arm 140 distal end passes through an arc that resides under the skin of the patient. Rotation of arm 140 is preferably accomplished with one or more pivoting tools, such as a percutaneous tool placed through the in-place cannula through which hinged assembly 120 was inserted. Detailed descriptions of a preferred percutaneous insertion method is described in reference to FIGS. 6A through 6H described herebelow. In the embodiment of FIG. 4B, both screw head 125 and screw head 155 include securing means, threads 127 and 157 respectively, into each of which a set screw, not shown, is placed to “lock in place” pivoting arm 140 and provide high levels of stabilization forces, including axial forces, radial forces and torsional forces. Threads 127 and 157 as well as the corresponding set screws, are configured to provide sufficient anti-rotation properties to prevent loosening over time, such as anti-rotation achieved with specific thread patterns and/or included adhesive. In an alternative embodiment, the engagement shown in FIG. 4B, without additional set screws into either threads 127 or threads 157, provides the necessary stabilization forces. In another alternative embodiment, an automatic anti-rotation mechanism engages when sufficient rotation of arm 140 is achieved, simplifying the procedure for the operator, such as by simplifying the placement of a set screw into threads 157 with an already locked in place pivoting arm 140.

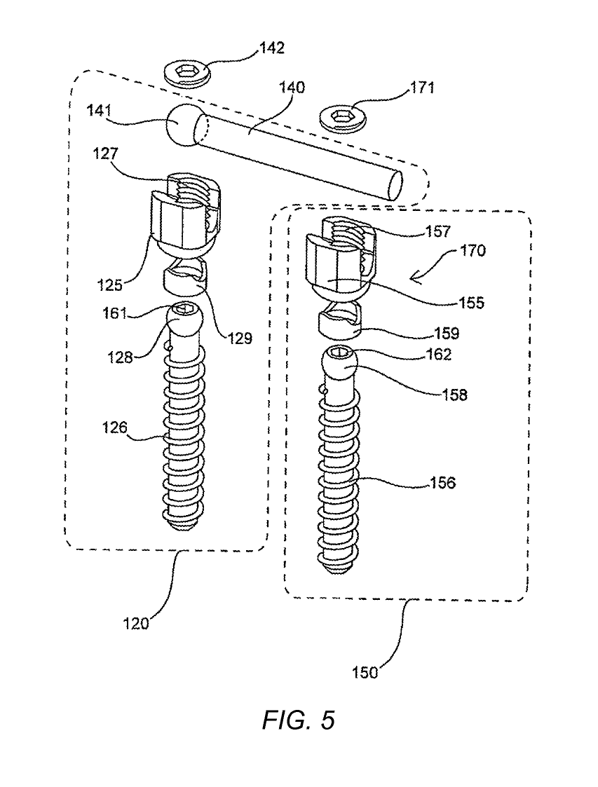

Referring now to FIG. 5, an exploded view of a preferred construction of the bone stabilization device of the present invention is provided. Hinged assembly 120 includes multiple components captured by the dashed line of FIG. 5. Pivoting arm 140 includes ball end 141 at its proximal end. Ball end 141 is sized and configured to be received by screw head 125 such that a rotatable hinge is formed, allowing the distal end of arm 140 to be rotated in numerous directions. Ball end 141 may be inserted by the operator, such as during a sterile procedure prior to insertion into the patient, or be provided pre-assembled by the manufacturer. Hinged assembly 120 further includes a bone anchor comprising an elongate tube with bone threads 126, ball end 128 and thru lumen 161, a lumen sized and configured to facilitate placement of hinged assembly 120 over a guidewire, such as a guidewire placed into a bone segment to be stabilized. Ball end 128 is sized and configured to be securedly engaged with pivoting element 129, which in turn securedly engages with screw head 125, such that polyaxial rotation of screw head 125 is achieved, such as rotation which simplifies insertion of hinged assembly 120 in a vertebra or other bone structure during an over-the-wire, through-a-cannula, percutaneous procedure.

The bone stabilization device of FIG. 5 further includes receiving assembly 150, also including multiple components captured by the dashed line of FIG. 5. Receiving assembly 150 includes cradle 170, an attachment point for the distal end of pivoting arm 140 of hinged assembly 120. Cradle 170 comprises screw head 155 that includes a “U” shaped groove for slidingly receiving the distal end of arm 140. In a preferred embodiment, the geometry of the “U” shape groove provides a snap fit to (permanently or temporarily) maintain the pivoting arm in place such as behind held in place during a further securing event. Receiving assembly 150 further includes a bone anchor comprising an elongate tube with bone threads 156, ball end 158 and thru lumen 162, a lumen sized and configured to facilitate placement of receiving assembly 150 over a guidewire, such as a guidewire placed into a bone segment to be stabilized. Ball end 158 is sized and configured to be securedly engaged with pivoting element 159, which in turn securedly engages with screw head 155, such that polyaxial rotation of screw head 125 is achieved, such as rotation which simplifies insertion of hinged assembly 120 in a vertebra or other bone structure during an over-the-wire, through-a-cannula, percutaneous procedure.

Screw head 155 of receiving assembly 150 includes means of securing the distal end of pivoting arm 140, threads 157 which are configured to accept a set screw after arm 140 is slidingly received by the groove of screw head 155, thus locking the distal arm in place. Set screw 171 can be inserted and engaged by an operator into threads 157, such as in an over-the-wire placement procedure through the lumen of screw 171 shown, Additional stabilization can be attained by inserting an additional set screw, set screw 142, into threads 127 of screw head 125 of the hinged assembly. Set screw 142 is also configured to be delivered in an open surgical procedure, or preferably an over-the-wire percutaneous procedure as placed through a similar lumen in screw 142. When threads 126 of hinged assembly 120 and threads 156 of receiving assembly 150 are anchored in bone, and pivoting arm 140 is secured within cradle 170, stabilization between hinged assembly 120 and receiving assembly 150 is achieved. In a preferred embodiment, pivoting arm 140 is configured to provide one or more of numerous parameter of stabilization, including but not limited to: rigid or fixed stabilization, and dynamic stabilization such as stabilization that allows controlled or limited motion in one or more directions. Pivoting arm 140 may be rigid, or have some degree of flexibility. Pivoting arm 140 may include one or more functional elements, such as a spring to resists but permits motion. Functional elements may include one or more engaging surfaces, such as surfaces that permit motion in one or more directions, yet limit motions in other directions, or surfaces which allow motion in a particular direction within a finite distance. Functional elements may provide other functions, such as an agent delivery element which provides an anti-infection agent or an agent targeted at reducing bone growth that otherwise would limit motion. These and other functions of pivoting arm 140 are described in detail in reference to subsequent figures herebelow.

Referring now to FIGS. 6A through 6H, a preferred method of stabilizing one or more patient bone segments, specifically vertebral segments, is illustrated. Referring to FIG. 6A, a guidewire placement procedure is illustrated in which a puncture has been made through the patient's skin 80, and into the pedicle 3a of patient vertebra 2. A guidewire 212, such as a K-wire, is shown in place, allowing subsequent devices to be passed over guidewire 212, using standard over-the-wire techniques. Referring now to FIG. 6B, a sequential dilation is being performed for the purpose of having a sufficiently sized cannula, dilating cannula 220, in place over guidewire 212. Dilating cannula 220 is positioned above, and with its central axis aligned with, vertebra 2 such that additional devices can be inserted over guidewire 212 and within a lumen of cannula 220 to access pedicle 3a and surrounding areas. The sequential dilation is performed to minimize tissue trauma that would result from initial insertion of the final, large sized cannula to be used.

Referring now to FIG. 6C, a cannulated drill bit 231 has been placed through cannula 220, over guidewire 212 and is in operable connection with cannulated drill 230. Drill bit 231 is near completion of drilling an appropriately sized hole into pedicle 3a of vertebra 2, such that an anchoring screw can be placed in a subsequent step. Referring now to FIG. 6D, cannulated drill bit 231 has been removed, using an over-the-wire removal or exchange technique, and receiving assembly 150 of the bone stabilization device of the present invention has been placed through cannula 220 and over guidewire 212. Receiving assembly 150 has been inserted with its bone anchoring portion and its attaching cradle 170 in an aligned, linear configuration. Guidewire 212 has been passed through a lumen, not shown but within both the anchoring portion and attaching cradle 170 of receiving assembly 150. In an alternative embodiment, guidewire 212 passes through a lumen of the anchoring portion, but then passes alongside attaching cradle 170 of receiving assembly 150. Receiving assembly 150 has been rotated, such as with a screwdriver tool or socket wrench tool passed through cannula 220 and engaging one or more portions of receiving assembly 150, tool not shown, such that its threads 156 are fully engaged with pedicle 3a of vertebra 2. In a preferred embodiment, these rotating tools include a thru lumen and are also inserted and manipulated over-the-wire.

Referring now to FIG. 6E, an adjacent vertebra, patient vertebra 4, has undergone similar access techniques, including guidewire placement, sequential dilation and pedicle drilling. As shown, receiving assembly 150 remains in place with threads 156 anchoring receiving assembly 150 to vertebra 2, and cradle 170 positioned to receive one or more pivoting arms of the present invention. Dilating cannula 220b has been inserted, such as the same cannula as previous figures or an additional cannula with cannula 220 remaining in place, not shown but as depicted in FIG. 6D. Guidewire 212b, preferably a K-wire, passes within cannula 220b, through the patient's skin 80 and into pedicle 3b of patient vertebra 4. Vertebra 4 is shown as an adjacent vertebra but in an alternative embodiment, vertebra 4 may be separated from vertebra 2 by one or more additional vertebrae, with the associated pivoting arm sized accordingly.

Referring back to FIG. 6E, cannula 220b is positioned above, and with its central axis aligned with, vertebra 4 such that additional devices can be inserted over guidewire 212b and within a lumen of cannula 220b to access pedicle 3b and surrounding areas. Hinged assembly 120 has been inserted with its bone anchoring portion, its pivoting arm 140 and hinge 130 in an aligned, linear configuration as shown. Prior to its insertion, hinged assembly 120 may have been assembled by the operator, such as an operator in the sterile field connecting the pivoting arm to the anchor portion, or may have been provided by the manufacturer in an assembled state. Guidewire 212b has been passed through a lumen, not shown but within both the anchoring portion and pivoting arm 140 of hinged assembly 120. In an alternative embodiment, guidewire 212b passes through a lumen of the anchoring portion, but then passes alongside attaching pivoting arm 140 of hinged assembly 120. Hinged assembly 120 has been rotated, such as with a screwdriver tool or socket wrench tool passed through cannula 220b and engaging one or more portions of hinged assembly 120, tool not shown, such that its threads 126 are fully engaged with pedicle 3b of vertebra 4. In a preferred embodiment, these rotating tools include a thru lumen and are also inserted and manipulated over-the-wire. In another preferred embodiment, the rotating tool includes an open lumen on its distal end sized to slide over the distal end of pivoting arm 140 and engage one or more engagable surfaces integral to hinged assembly 120 and located at or near hinge 130.

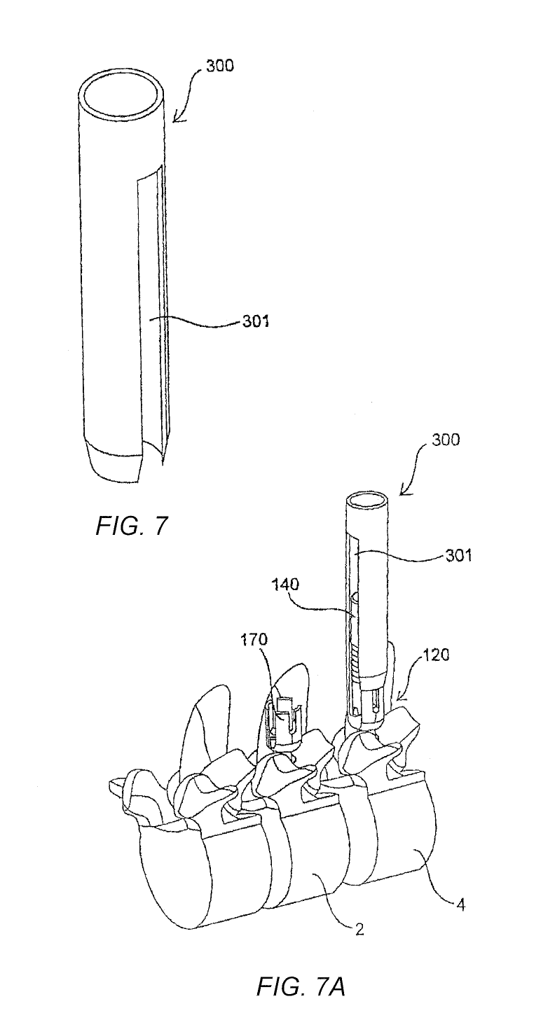

Referring now to FIG. 6F, hinged assembly 130 is securely attached to vertebra 4, a pivoting arm 140 is being rotated, such that the distal end of arm 140 forms an arc that remains under patient's skin 80, and is slidingly received into a groove of attaching cradle 170 of receiving assembly 150. Pivoting arm 140 may rotatably pass through a slot in cannula 220b, not shown but described in detail in reference to FIGS. 7 and 7A. Alternatively, cannula 220b can be retracted a sufficient distance to allow pivoting arm 140 to swing below the distal end of cannula 220b. In the embodiment shown in FIG. 6F, guidewire 212b has been removed to allow pivoting arm 140 to freely swing toward cradle 170. In an alternative embodiment, pivoting arm 140 includes a slot from its thru lumen to it's outer surface such that arm 140 can be pivoted away from a guidewire. In another alternative embodiment, hinged assembly 120 is inserted such that pivoting arm 140 is not over-the-wire, i.e. does not include a guidewire lumen and is inserted with pivoting arm alongside the guidewire. In this embodiment, arm 140 can also be rotated with the guidewire in place.

Referring now specifically to FIG. 6G, a percutaneous screwdriver 240 of the present invention has been inserted within the lumen of cannula 220b and is rotatably engaging a set screw, now shown but as has been described in reference to FIG. 5 hereabove, to secure pivoting arm 140 to prevent or limit rotation. In a preferred embodiment, screwdriver 240 and inserted set screws include lumens such that each can be inserted over an in-place guidewire. In another preferred embodiment, not shown, percutaneous screwdriver 240 is similarly inserted within the lumen of cannula 220, not shown but aligned with receiving assembly 150 as shown in FIG. 6D, such that another engaging set screw can be inserted, into cradle 170, to securedly attach pivoting arm 140 to cradle 170. Referring now to FIG. 6H, the cannulae and guidewires have all been removed, and bone stabilization device 100 is implanted in the patient. Receiving assembly 150 is securedly attached to vertebra 2, and hinged assembly 120 is securedly attached to vertebra 4. Pivoting arm 140 is securedly attached to receiving assembly 150 thus providing stabilization between vertebra 2 and vertebra 4. The type and amount of stabilization achieved between the two vertebrae can take on the various forms described throughout this application, including but not limited to: fixed or fused stabilization, and dynamic stabilization.

Referring now to FIG. 7, a slotted cannula of the present invention is illustrated. Slotted cannula 300, preferably a sequential dilating cannula, additional sliding tubes not shown, includes a longitudinal slot, starting from its distal end, the end that is inserted into the patient, and extending proximally. Slot 301, and any additional slots included in any slidingly received tubes not shown, are sized and positioned such that a device contained within cannula 300 can be passed through the slot, such as to a location within the body of a patient. Referring now to FIG. 7A, slotted cannula 300 is shown passing through the skin of a patient, skin not shown, and aligned with vertebra 4 of the patient. Hinged assembly 120 of the present invention is included within the lumen of cannula 300 and has been securedly attached to vertebra 4. Also shown is the receiving assembly of the present invention with attaching cradle 170 having been securedly attached to vertebra 2 of the patient. Slot 301 of cannula 300 has been aligned such that pivoting arm 140 of hinged assembly 120 can be rotated to the orientation in which the distal end of arm 140 is slidingly received by the groove of cradle 170 without having to reposition cannula 300. In a preferred embodiment, the proximal end of slotted cannula 300 includes one or more markings that indicate the location of slot 301 such than when inserted in the body, slot 301 position can be oriented and/or confirmed. In an alternative embodiment, dilator 300 includes multiple slots along its length.

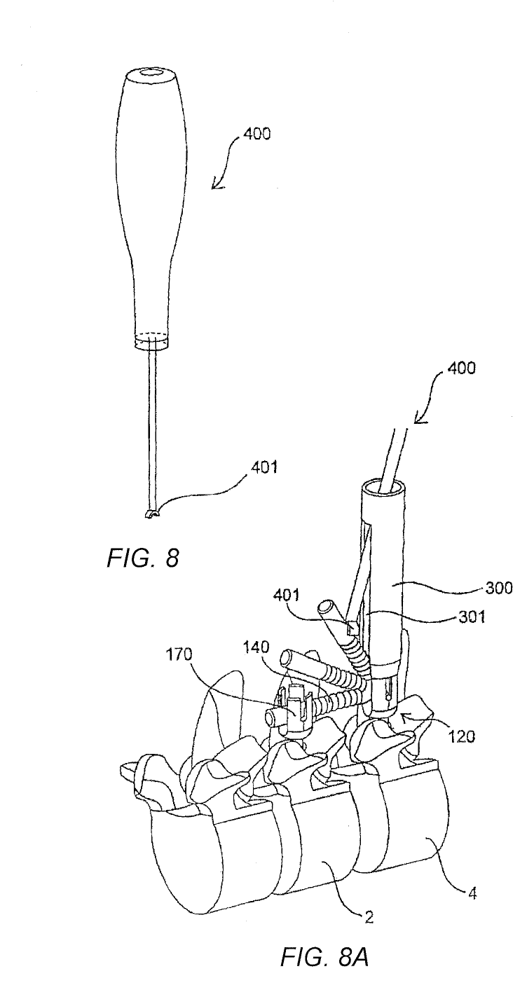

Referring now to FIG. 8, a pivoting tool of the present invention is illustrated. Pivoting tool 400 includes engagement end 401, configured to operably engage a pivoting arm of the present invention, such as to rotate the pivoting arm through one or more cannulae during a percutaneous procedure. Referring now to FIG. 8A, slotted cannula 300 is shown passing through the skin of a patient, skin not shown, and aligned with vertebra 4 of the patient. Hinged assembly 120 of the present invention is included within the lumen of cannula 300 and has been securedly attached to vertebra 4. Also shown is the receiving assembly of the present invention with attaching cradle 170 having been securedly attached to vertebra 2 of the patient. Slot 301 of cannula 300 has been aligned such that pivoting arm 140 of hinged assembly 120 can be rotated using pivoting tool 400 to the orientation in which the distal end of arm 140 is slidingly received by the groove of cradle 170. Pivoting arm 140 is rotated by first engaging end 401 of pivoting tool 400 with arm 140, and then advancing and potentially pivoting end 401 until arm 140 is engaged with cradle 170. In a preferred embodiment, the proximal end of pivoting tool 400 includes one or more markings that indicate the orientation of engaging end 401, such as when engaging end 401 has an non-symmetric geometry.

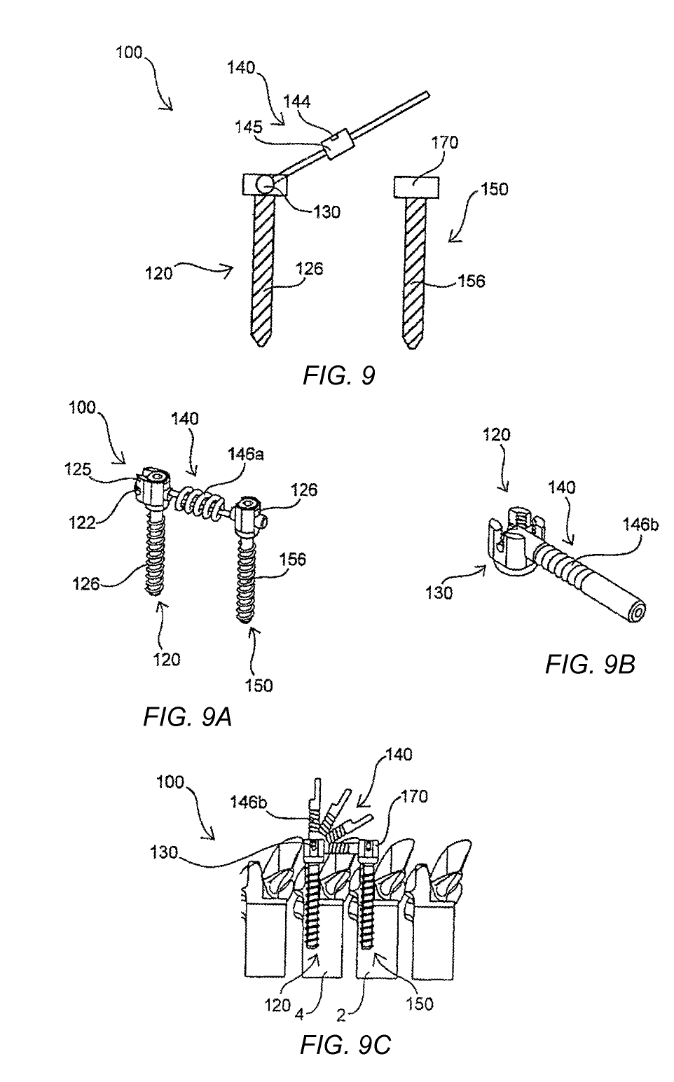

Referring now to FIG. 9, another preferred embodiment of the bone stabilization device of the present invention is illustrated. FIG. 9 depicts a schematic view of bone stabilization device 100 comprising hinged assembly 120 and receiving assembly 150. Hinged assembly 120 includes a bone anchoring portion including bone threads 126, that is fixedly or rotatably attached to hinge 130. Hinge 130 provides a rotatable connection, such as a single or multi-axis rotatable connection, to pivoting arm 140. Receiving assembly 150 includes a bone anchoring portion including bone threads 156, that is fixedly or rotatably attached to cradle 170. Cradle 170 is configured to be securedly attached, intraoperatively, to pivoting arm 140 to achieve stabilization between a first bone location and a second bone location. The type and amount of stabilization can be greatly specific and customized as is provided in the multiple embodiments of the present invention.

As depicted in the schematic representation of FIG. 9, pivoting arm 140 includes functional element 145, depicted at the midpoint of pivoting arm 140 but existing anywhere along its length or comprising the entirety of pivoting arm 140. Also included in pivoting arm 140 is adjustment means 144, shown as part of functional element 145 but alternatively a separate component or components of functional element 145. Adjustment means 144 is an engageable assembly, preferably engageable via cannulae as has been described in reference to FIGS. 6A through 6H, placed during the procedure implanting bone stabilization device 100 or a subsequent procedure in which bone stabilization device 100 is to be adjusted. Numerous parameters of device 100 may require adjustment, at the time of implantation or thereafter, including but not limited to: force adjustments such as forces resisting translation, rotation and bending of vertebral segments; length adjustments; position adjustments; and combinations thereof. In a preferred embodiment, pivoting arm 140 is slidable within a component of device 100 or includes two slidable arms, and adjustment means 144 is a screw driven assembly that causes controlled sliding and resultant length adjustment of pivoting arm 140. In another preferred embodiment, device 100 includes one or more springs which provide compressive forces for stabilization, and adjustment means 144 is a screw driven assembly to adjust the forces exerted by the springs. In yet another preferred embodiment, device 100 includes one or more pneumatic or hydraulic assemblies and adjustment means 144 is a screw driven assembly to adjust those assemblies.

Functional element 145 can provide functions that enhance therapeutic benefit and/or reduce complications and adverse side effects. In a preferred embodiment, functional element 145 comprises one or more flexible joints and provides dynamic stabilization to mimic a health joint such as a vertebral segment. In another preferred embodiment, functional element 145 comprises an artificial facet or partial facet, and serves the function of replacing or supporting a facet of a patient's vertebral segment. In yet another preferred embodiment, functional element 145 provides a function selected from the group consisting of: single axis flexion; multi-axis flexion; force translation such as providing a force to hinder motion in one or more directions; motion limiting such as limiting a maximum relative motion between the first location and the second location; agent delivery such as anti-bone proliferation drugs; radiation delivery percutaneous access; facet replacement; facet enhancement; and combinations thereof. In yet another preferred embodiment, functional element 145 provides multiple functions such as those described above. Drug delivery or radiation exposure might be advantageous to limit the body's reaction to the surgery and/or the implant, such as bone proliferation which may limit joint movement that has been dynamically stabilized. Drug delivery, such as a coating on one or more components of device 100, or an eluding drug depot such as a refillable drug depot integral to functional assembly 145 or another component, may alternatively or additionally be used to deliver an agent such as an anti-biotic delivered to prevent infections not uncommon to implants and implant procedures. In another preferred embodiment, functional element 145 is a flexible band, such as a band that provides a tensioning force between the two bone locations to be stabilized. In another preferred embodiment, the band is included to provide a ligament function. In yet another preferred embodiment, functional element 145 provides multiple functions, such as two or more functions selected from the numerous functions described immediately hereabove.

In another preferred embodiment, device 100 includes a valve assembly, such as a valve assembly integral to adjustment means 144. The valve assembly can be used to provide one-way fluid access to one or more components of device 100, such as to refill a drug depot, adjust a hydraulic or pneumatic assembly, or other valve function. In an alternative embodiment, a valve is included which opens at a pre-determined pressure, such as a pressure relief valve which opens to prevent undesirable forces from being generated by device 100.

Referring now to FIG. 9A, a bone stabilization device of the present invention is depicted with a functional element configured to provide dynamic stabilization. Hinged assembly 120 includes axle 122, a pin projecting from pivoting arm 140 that is captured and rotatably received a receiving hole in screw head 125 to form a single degree of freedom hinge. Pivoting arm 140, shown secured with set screws to cradle 170 of receiving assembly 150, includes a functional element along its length, torsion-compression spring 146a that is configured to provide appropriate torsion and compressive forces for dynamic stabilization of two bone structures.

Referring now to FIG. 9B, another preferred hinge assembly of the present invention is depicted. Hinge assembly 120 includes hinge 130, of similar construction to the hinge of FIG. 9A, and pivoting arm 140, which includes a functional element, compression spring 146b along its length. Compression spring 146b is configured to provide appropriate forces for dynamic stabilization of two bone structures when hinge assembly 120 and pivoting arm 140 are securedly attached to a receiving assembly of the present invention.

Referring now to FIG. 9C, device 100, consisting of the hinge assembly 120 of FIG. 9B, is shown secured to vertebra 4 of a patient. Also implanted is receiving assembly 150 shown secured to vertebra 2 of the patient. Pivoting arm 140 is shown in various rotational positions, rotating clockwise, as shown, until fully engaged with cradle 170. Pivoting arm 140 includes compression spring 146b along its length to provide dynamic stabilization between vertebra 4 and vertebra 2 of the patient.

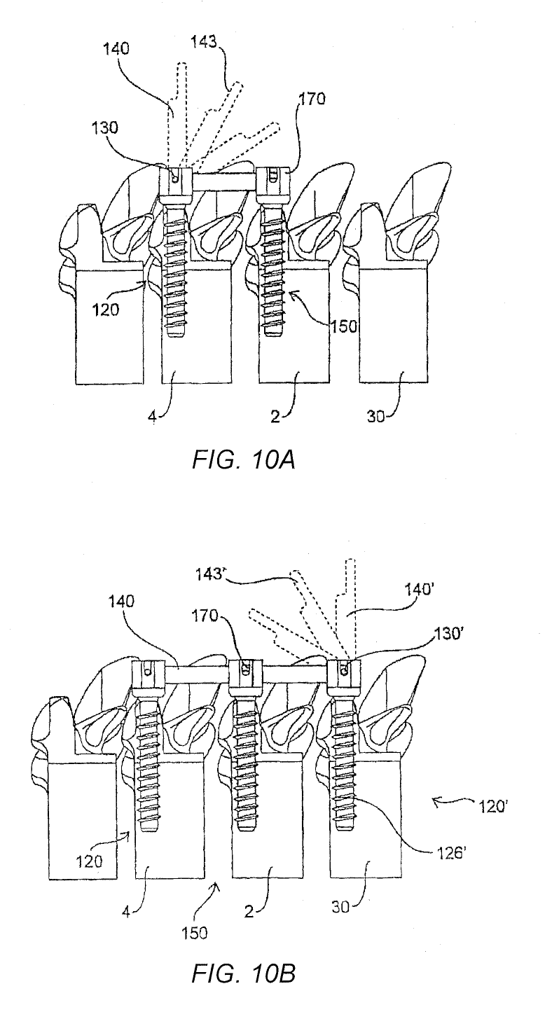

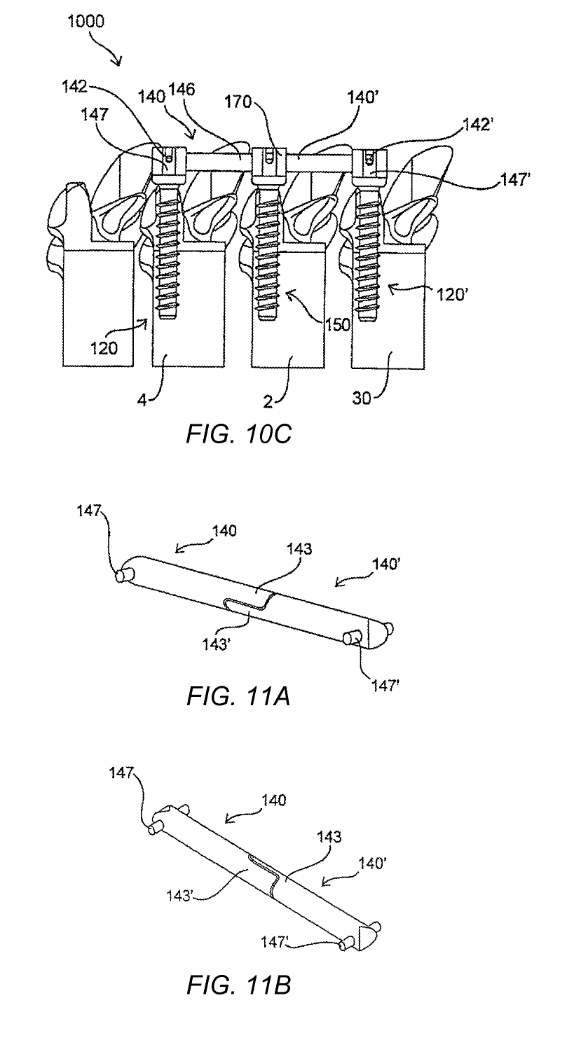

Referring now to FIGS. 10A, 10B and 10C, another preferred device and method of the present invention is illustrated in which three vertebral segments are stabilized relative to each other. Referring specifically to FIG. 10A, a hinged assembly 120 has been securedly attached to vertebra 4 and a receiving assembly 150 has been securedly attached to adjacent vertebra 2, such as by using similar percutaneous tools and techniques described in reference to FIGS. 6A through 6H. Pivoting arm 140 is being rotated in a clockwise direction, as shown, via hinge 130, to a location in which its distal end resides within cradle 170 of receiving assembly 150. In the preferred embodiment of FIGS. 10A and 10B, the distal end of pivoting arm 140 includes a reduced segment, recess 143, which is configured to geometrically mate with an end portion of a separate pivoting arm. Referring now to FIG. 10B, a second hinged assembly, hinged assembly 120′ has been inserted into a vertebra 30, a vertebra adjacent to vertebra 2 but opposite the side adjacent to vertebra 4, such as by using similar percutaneous tools and techniques described in reference to FIGS. 6A through 6H. Hinged assembly 120′ is shown with its pivoting arm 140′ being rotated in a counterclockwise direction, as shown, via hinge 130′ to a location in which it's distal ends also resides within cradle 170 of receiving assembly 150. The distal end of pivoting arm 140′ also includes a reduced segment, recess 143′, which is configured to geometrically mate with the end portion of recess 143 of pivoting arm 140 of hinged assembly 120.

Referring now specifically to FIG. 10C, poly-segment (more than two segments) bone stabilization device 1000 includes first hinged assembly 120, second hinged assembly 120′ and receiving assembly 150. Receiving assembly 150 has slidingly receiving and is not securedly attached to the distal ends of pivoting arm 140 and pivoting arm 140′ or hinged assembly 120 and hinged assembly 120′ respectively. Stabilization, such as dynamic stabilization or fixed stabilization, has been achieved between vertebra 4 and vertebra 2 and vertebra 30. The numerous enhancements, such as functional elements including one or more spring included in a pivoting arm, or other enhancements, can be included in first hinged assembly 120, second hinged assembly 120′ and/or receiving assembly 150 to provide more therapeutic benefit, improve safety and/or longevity of the implanted device.

The distal ends of the pivoting arms 140 and 140′ each have a reduced segment such that the combined cross-sections is relatively equivalent to the cross-section of either arm prior to the reduction. This mating portion allows a similar cradle 170 to be used that would be used to securedly engage a single pivoting arm without a reduced segment. Various geometries of the reduced cross sections can be employed. In a preferred embodiment, a fixation means, such as a set screw, not shown, is placed through each reduced portion and into cradle 170 to secure both pivoting arms to the receiving assembly.

Referring now to FIGS. 11A and 11B, two preferred geometries of the reduced portions of FIGS. 10A through 10C are illustrated. A pair of pivoting arms is shown, pivoting arm 140 and pivoting arm 140′. On each proximal end, a pin, axle 147 and axle 147′ extends radially out from the tubular structure, each pin configured to rotate in a bushing of the appropriate hinge assembly to perform a hinge function. FIG. 11A represents a geometry including two half-circular cross sections that are stacked on top of each other, when engaged, as viewed from the top of the cradle (looking down on the anchoring means). FIG. 11B represents a geometry also consisting of two half-circular cross sections, these sections aligned in a side-by-side orientation as viewed from the top of the cradle.

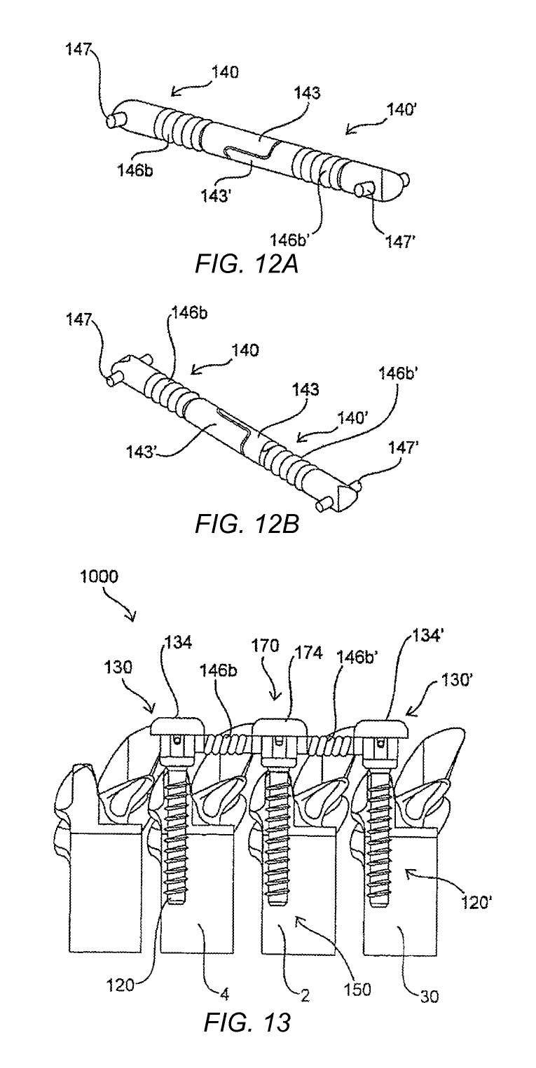

Referring now to FIGS. 12A and 12B, two additional preferred geometries of pairs of pivoting arms are illustrated. The cross sectional geometries of pivoting arms 140 and 140′ are the same as those of arms 140 and 140′ of FIGS. 11A and 11B respectively. The pivoting arms of FIGS. 12A and 12B further each include a functional element, coil springs 146b and 146b′, along their length, to provide dynamic stabilization forces when a poly-segment stabilization device of the present invention is implanted. Referring now to FIG. 13, poly-segment bone stabilization device 1000 includes first hinged assembly 120 and second hinged assembly 120′ which include the pivoting arms 140 and 140′ of FIGS. 12A and/or 12B. In the preferred embodiment of FIG. 13, multiple caps are placed on engagable portions of components of device 1000, such as cap 134 placed on top of the hinge of hinged assembly 120, cap 174 placed on top of cradle 170 of receiving assembly 150, and cap 134′ placed on top of the hinge of hinged assembly 120′. These caps are made of a biocompatible metal or plastic, and prevent tissue in-growth and other contamination from entering engagement means such as slots and other engagable surfaces. The caps are preferably a pressure fit or screw cap, and can be easily removed with minimally invasive means. In an alternative embodiment, one or more of the caps are biodegradable.

Referring now to FIGS. 14A, 14B and 14C, hinge mechanisms of the hinged assemblies of the present invention are illustrated. Referring specifically to FIG. 14A, an operator assembled hinge is illustrated. Hinge 130 includes a projecting pin, axle 147, that extends from pivoting arm 140. Axle 147 is configured to be snapped in place into slot 131 of screw head 125. Screw head 125 is fixedly or rotatably connected to an anchoring portion of hinge assembly 120, anchor portion not shown. Screw head 125 further includes threads 127, which are configured to accept a set screw to prevent inadvertent disassembly of hinge 130. Threads 127 can also be used to lock-down, or otherwise prevent rotation of arm 140. A set can be partially inserted to capture the pin yet allow rotation, such as prior to implantation in the patient, or a set screw can be inserted after insertion into the body of the patient.

Referring specifically to FIG. 14B, another preferred embodiment of a hinge of the present invention is illustrated. Hinged assembly 120 includes pivoting arm 140, which is pivotally attached to base 124 via hinge 130. Pivoting arm 140 includes a projecting pin 147, which is permanently captured by a bushing included in housing 132. Pivoting arm 140 can be fixed in place by one or more mechanisms described in detail throughout this application.

Referring specifically to FIG. 14C, an alternative embodiment of a hinge is provided in which a portion of pivoting arm 140 includes a flexible portion, such as two metal rods connected with a elastic or otherwise deformable section. Pivoting arm 140 is fixedly mounted to base 124, and hinge 130 consists of flex point 139 of arm 140. Pivoting arm 140 and flex point 139 may be resiliently biased, either in the final secured position, or starting (linearly aligned with the anchor portion) position, or a position in between. Alternatively, pivoting arm 140 may be plastically deformable, changing its biased position as it is rotated.

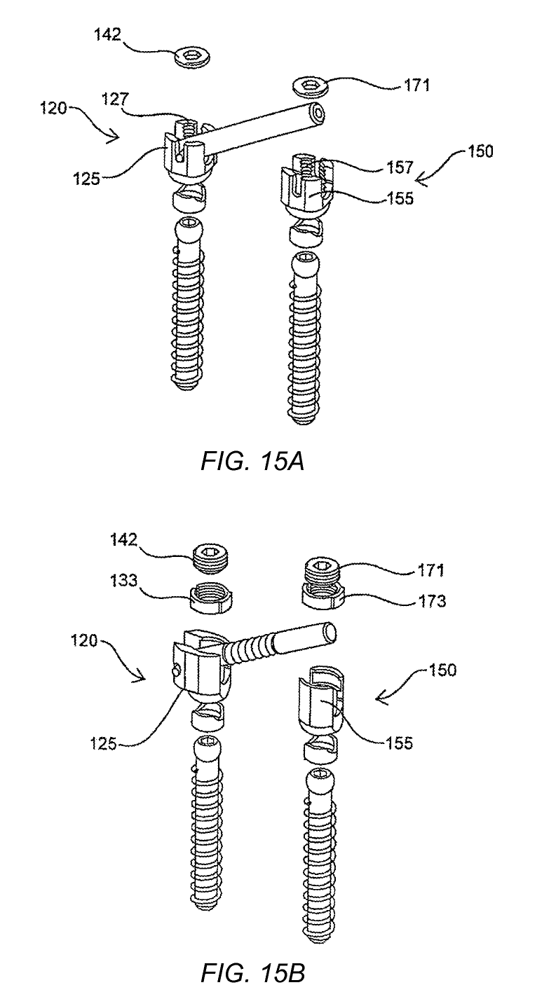

Referring now to FIGS. 15A and 15B, means of securing the pivoting arm of the present invention are illustrated. FIG. 15A illustrates sets screws 142 and 171, configured to be operatively engaged with threads 127 and 157 respectively. Threads 127 are integral to screw head 125 of hinged assembly 120 and threads 157 are integral to screw head 155 of receiving assembly 150. Both screw 142 and 171 include a thru-lumen, which allows over-the-wire insertion, such as insertion performed by an operator using an over-the-wire screwdriver of the present invention. Referring now to FIG. 15B, an alternative securing means is illustrated, including a two-piece assembly comprising a screw and an expandable ring. Ring 133 is inserted to screw head 125 of hinge assembly 120 after which screw 142 is rotatably engaged with ring 133, causing ring 133 to radially expand and provide a high compression, reliable connection. Similarly, ring 173 is inserted into screw head 155 of receiving assembly 150 after which screw 171 is rotatably engaged with the threads of ring 173, causing ring 173 to radially expand and provide high compression, reliable connection.

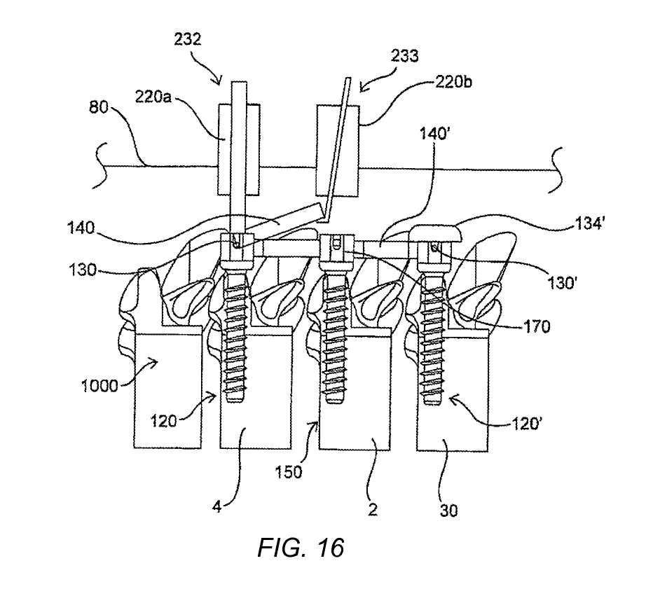

Referring now to FIG. 16, a method of accessing a bone stabilization device is illustrated. Two cannula, cannula 220a and 200b are shown as having been inserted through the patient's skin 80 at locations directly above vertebra 4 and vertebra 2 respectively. A poly-segment hinged assembly device 1000 of the present invention has been planted at an earlier date, such as a time period of months or more earlier. Device 1000 is configured to stabilize vertebra 4, vertebra 2 and vertebra 30 in a fixed or fused configuration, or in a dynamically stabilized configuration. Device 1000 includes a first hinged assembly 120 securedly attached to vertebra 4, a receiving assembly 150 securedly attached to vertebra 2 and a second hinged assembly 120 securedly attached to vertebra 30. Pivoting arm 140′ of hinged assembly 120′ is shown in secure attachment with cradle 170 of receiving assembly 150. Hinge 130′ is covered with cap 134′ attached during the original implantation procedure of device 1000. Caps that were originally attached in the original implantation procedure, such as a cap on hinge assembly 130 and cradle 170 have been removed in the accessing procedure of FIG. 16. Percutaneous grasping and ply tools, as well as percutaneous rotational tools such as screwdrivers are preferably used to detach these caps and extract through either cannula 220a or 220b.

The method depicted in FIG. 16 involves the unsecuring of pivot arm 140, already completed, and the reverse rotation of pivot arm 140, depicted as partially rotated by using lifting tool 233 inserted through cannula 220b. Screwdriver 232 has been inserted through cannula 220a and used to loosen and/or remove engagement means such that pivoting arm 140 can rotate, engagement means already removed and not shown. Subsequent steps may include the complete removal of hinge assembly 120, and reinsertion of a new hinged assembly, such as when hinged assembly 120 is damaged or when a hinged assembly with different properties, such as a differently configured pivoting arm 140 is desirable. In an alternative embodiment, hinge 120 is adjusted, and pivoting arm 140 again secured to cradle 170. Numerous combinations of adjustments and replacements of one or more components of system 1000 can be accomplished utilizing the percutaneous tools and methods depicted in FIG. 16. Use of one or more caps, such as cap 134′, make subsequent engagement of tools with system 1000 components easier to accomplish since the covered surfaces are free from material that would compromise engagement.

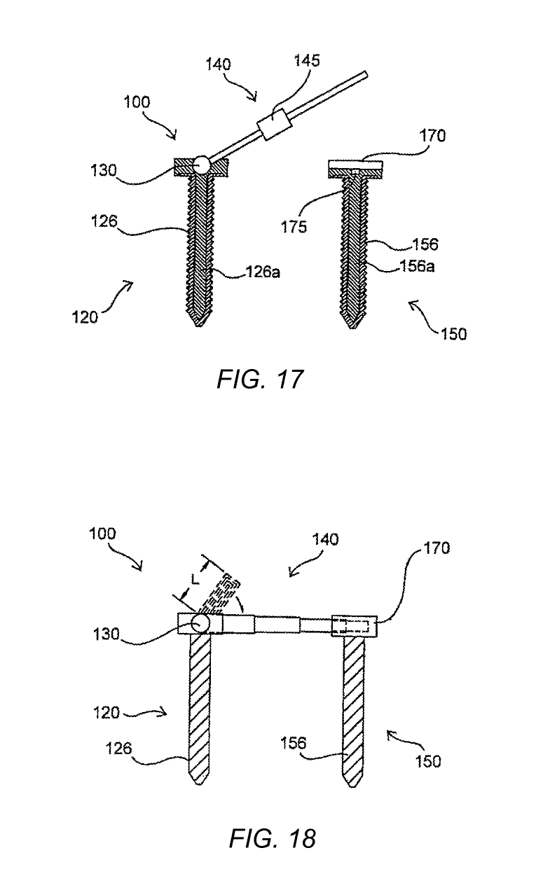

Referring now to FIG. 17, another preferred embodiment of bone stabilization device of the present invention is illustrated wherein anchor portions consist of an outer tube and a removable core. Device 100 includes hinged assembly 120 including a bone anchor and pivoting arm 140 which attaches to the bone anchor portion via hinge 130. Pivoting arm 140 includes function element 145, such as a spring or other flexible element that provides a flexion point for dynamic stabilization of two bone structures. Device 100 further includes receiving assembly 150 which includes a bone anchor portion which is attached to surface 170. Surface 170 is configured to securedly attach to the distal end of pivoting arm such as via a screw, not shown, but preferably inserted through the distal end of arm 140 and into threads 175. Both hinged assembly 120 and receiving assembly 150 include anchor portions which have external threads for engaging and securing in bone, and a removable inner core, configured to be removed via one or more means such as the threaded engagement depicted in FIG. 17. Internal threads 126a and internal threads 156a of the hinged assembly and receiving assembly anchor portions respectively, allow the remaining portion of these assemblies to be removed, such as after a period of implantation, while leaving the outer threaded portions in place, such as for insertion of a subsequent assembly or otherwise.

Referring now to FIG. 18, another preferred embodiment of the bone stabilization device of the present invention is illustrated wherein the pivoting arm can be telescopically extended or retracted, such as to rotate with a minimal radius of curvature. Device 100 includes hinged assembly 120 including a bone anchor and pivoting arm 140 which attaches to the bone anchor portion via hinge 130. Device 100 further includes receiving assembly 150 which includes a bone anchor portion which is attached to cradle 170. Cradle 170 is configured to securedly attach to the distal end of pivoting arm such as by the various engagement means described throughout this application. Both hinged assembly 120 and receiving assembly 150 include anchor portions which have external threads for engaging and securing in bone, external threads 126 and 156 respectively. Pivoting arm 140 consists of a series of interlocking slidable tubes configured to telescopically be advanced, such as to be long enough to engage with cradle 170. In a preferred embodiment, hinged assembly 120 is percutaneously inserted into the body, and pivoting arm 140, in a telescopically retracted state, is pivoted an amount such that it's axis is pointing at the engagement portion of cradle 170, such as a ninety degree rotation in the configuration shown. Subsequently, using a push tool, an integral extending assembly such as a hydraulic or pneumatic extending assembly, or other means, the distal end of an inner, such as the innermost, telescopic section is advanced until properly seated for engagement in cradle 170. The telescoping tubes of pivoting arm 170 are preferably made of a rigid metal, sufficient to provide sufficient force to achieve the desired stabilization.

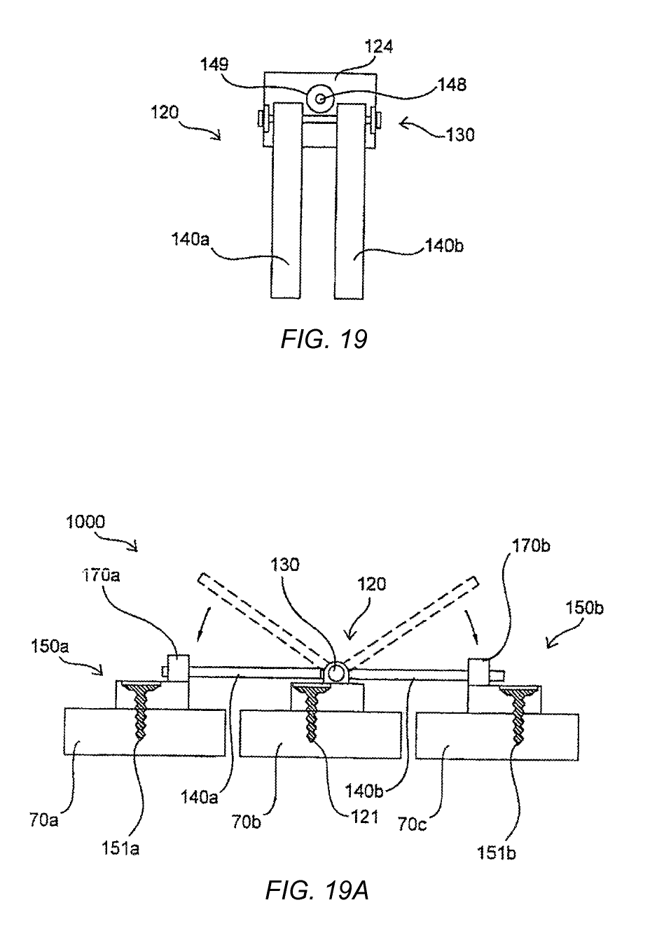

Referring now to FIG. 19, a preferred embodiment of the hinged assembly of the present invention is illustrated wherein multiple pivoting arms are included. Hinged assembly 120 includes thru lumen 148, such as a lumen for a guidewire and/or bone screw, and recess 149 which can accommodate the screw head of such a bone screw. Hinged assembly 120 further includes hinge 130, which rotatably attaches base 124 to two pivoting arms, 140a and 140b. In an alternative embodiment, more than two pivoting arms are rotatably attached by hinge 130. These multiple arms can be used to stabilize the particular bone segment to which hinged assembly 120 is attached to a single additional bone segment, or multiple bone segments wherein each arm is connected by an operator to a component on the different bone segments. Referring now to FIG. 19A, a preferred configuration of a poly-segment stabilization device 1000 and attachment method is illustrated. Device 1000 includes the dual arm hinged assembly 120 of FIG. 19, and two receiving assemblies 150a and 150b. Hinged assembly 120 is securedly attached via screw 121 to second bone segment 70b, such as a fractured bone in the patient's arm or leg, or a vertebra of the patient's spine. Receiving assembly 150a is securedly attached to bone segment 70a with screw 151a and receiving assembly 150b is securedly attached to bone segment 70c with screw 151b, the three bone segments aligned as shown. Hinged assembly 120, preferably inserted in the over-the-wire percutaneous technique described in reference to FIGS. 6A through 6H, such as wherein one or none of the pivoting arms includes a thru lumen for advancement of the percutaneous guidewire. As shown, pivoting arm 140a is rotated such that it can be securely engaged with cradle 170a of receiving assembly 150b and pivoting arm 140b is rotated such that it can be securely engaged with cradle 170b of receiving assembly 150b. Upon dual engagement of each pivoting arm, fixed or dynamic stabilization is achieved between the three bone segments, 70a, 70b and 70c. Additional dual arm and single arm hinged assemblies, as well as dual or single cradle receiving assemblies, can be added, in the linear arrangement shown, and/or with hinged assemblies and/or receiving assemblies placed in a side-by-side configuration. These poly-component (more than 2) devices and methods can be useful in treating complex bone fractures and other poly-location stabilization procedures. In an alternative embodiment, the multiple arms of the hinged assembly have different lengths, such as to securedly engage with components separated from the hinged assembly by different displacements. Each of the multiple arms can rotate to a single receiving assembly, or different receiving assemblies.

Referring now to FIGS. 20, 20A and 20B, a preferred embodiment of the present invention is illustrated wherein the receiving assembly automatically engages the pivoting arm of the hinged assembly. Referring specifically to FIG. 20, an end view of hinged assembly 150 is shown wherein cradle 170 is securedly mounted to plate 154, via fixed or movable engagement means. Cradle 170 includes a circular notch for maintaining a pivoting arm of the present invention, the diameter chosen to be slightly larger than the diameter of the appropriate pivoting arm. At the top of the notch is projection 176, wherein the size of notch 176 and the materials of construction of cradle 170 are chosen such that the distal end of a pivoting arm can snap into place, being maintained in place by projection 176 under certain load conditions. In a preferred embodiment, the forces are chosen such that no additional securing means are required to achieve the desired therapeutic function (stabilization of bone structures). In an alternative, also preferred embodiment, an additional securing function is included, such as the retraining set screws described throughout this application. Referring to FIG. 20A, pivoting arm 140 of hinged assembly 120 is shown rotating in a clockwise direction about hinge 130. Receiving assembly 150, of FIG. 20, is included and provides a snap-fit function that retains the distal end of arm 140 when full rotated to be constrained within cradle 170 as shown in FIG. 20B.

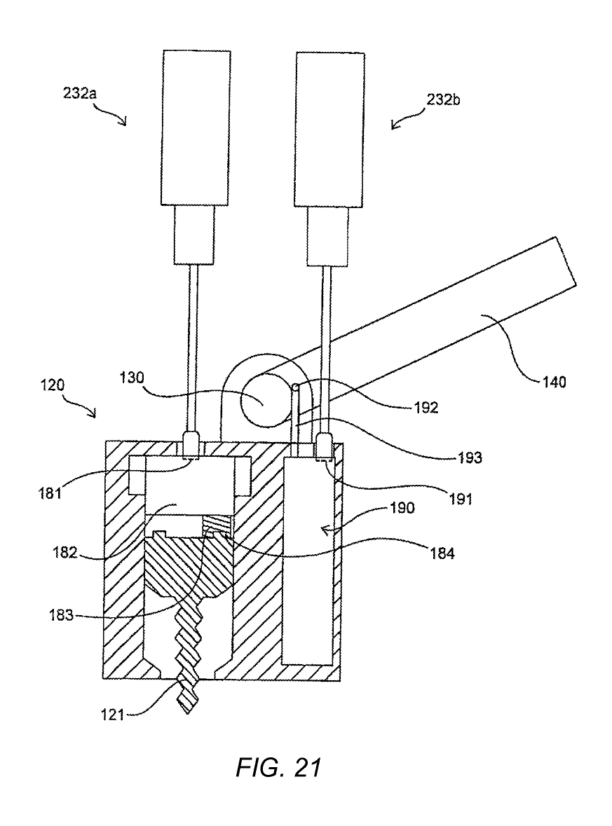

Referring now to FIG. 21, a preferred embodiment of the hinged assembly of the present invention is illustrated wherein assemblies are included that provide a mechanical advantage to perform one or more functions, such as functions performed during or post implantation. Hinged assembly 120 includes pivoting arm 140, which is rotatably attached to hinge 130. Pivoting arm 140 is also rotatably attached to piston 193 via pin 192. Piston 193 is a hydraulically or pneumatically driven piston of piston assembly 190. Piston assembly 190 includes engagable activation means 191, shown in operable attachment to screwdriver 232b, such as a percutaneous screwdriver than can be advanced through a percutaneous cannula. Rotation of means 191 is used to advance and retract piston 193, which in turn causes pivoting arm 140 to rotate in counterclockwise and clockwise directions, respectively. Hydraulic and pneumatic assemblies can be used to generate large amounts of force, perform precise movements, and provide other mechanical advantages.

Hinged assembly 120 further includes another mechanical advantage assembly, a precision, high-torque screw advancement and/or screw retraction assembly including linear advancement element 182, rotational element 183, and engagement means 181. The screw advancement assembly is shown as engaged by percutaneous screwdriver 232a on its input end, and engages screw 121, preferably a screw configured for advancement into bone, such as a screw with polyaxial head pedicle screw construction. Linear advancement element 182 includes an expandable bellows construction, expandable via an internal gear train mechanism, not shown, such that as screwdriver 232a is engaged and rotated, the bottom surface of element 182 expands in the direction opposite the surface including hinge 130. Rotation element 182 is operably engaged with a circular array of teeth integral to screw 121, teeth 184. Rotation of screwdriver 232a when engaged with engagement means 181 causes both downward expansion of element 182, and rotation of screw 121 via rotational element 182's engagement with teeth 184. Configuration of the included gear train can provide numerous benefits, including but not limited to: high levels of torque; precise advancement and/or rotation of screw 121; and other advantages.

It should be appreciated that numerous forms and varied configurations of mechanical advantage assemblies can be incorporated, to provide one or more functions, especially to overcome the limitations imposed by small implantable assemblies that are preferably accessed with miniaturized tools. Hydraulic and pneumatic assemblies can be employed to generate large forces and provide other benefits. Gear trains and lever arm assemblies can be employed to create precision control of motion and also provide other benefits. These mechanical advantage assemblies of the present invention can be integrated into one or more components of the bone stabilization device, such as the hinged assembly, the receiving assembly, or a separate component also configured to be implanted. These mechanical advantage assemblies can perform numerous functions including but not limited to: rotation of the pivoting arm; extension such as telescopic extension of the pivoting arm such as a hydraulically advanced pivoting arm; rotation and/or longitudinal advancement of a bone anchoring component such as a bone screw, application of one or more forces to a bone segment, such as a variable force stabilizing function such as a shock absorber for two bone segments; and combinations thereof.

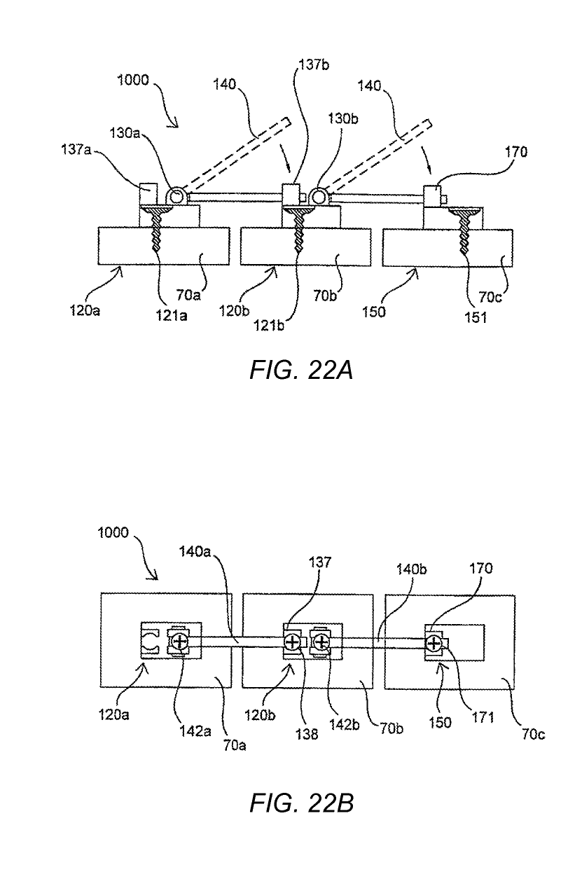

Referring now to FIGS. 22A and 22B, another poly-segment bone stabilization device and method of the present invention is illustrated, in which two hinged assemblies are implanted at adjacent locations, and at least one hinged assembly includes an attaching cradle for receiving a pivoting arm of the other hinged assembly. System 1000 includes first hinged assembly 120a securedly attached to first bone segment 70a via attachment screw 121a, second hinged assembly 120b attached to second bone segment 70b via attachment screw 121b, and receiving assembly 150 attached to third bone segment 70c via attachment screw 151. Bone segments 70a, 70b and 70c, such as three adjacent vertebra of a patient, receive device 1000 in order to provide stabilization between the segments. Both hinged assembly 120a and 120b include means of receiving a pivoting arm, the receiving means comprising cradles 137a and 137b respectively. In the figure shown, hinged assembly 120b receives, in cradle 137b, the pivot arm of hinged assembly 120a. Cradle 137a of hinged assembly 130a is implanted with no secured pivoting arm, an acceptable configuration especially as it would result in fewer variations of components (hinged assemblies with and without cradles).

The pivoting arm of hinged assembly 120b is received by cradle 170 of receiving assembly 150 as shown. Each of the receiving arms can provide fixed or dynamic stabilization, through inclusion of one or more flexing means as has been described in detail hereabove. In an alternative embodiment, a single component, a universal component consisting of a hinged assembly with a cradle, and a detachable (or attachable) pivoting arm, can be used, in multiplicity, to recreate the three-segment scenario depicted in FIGS. 22A and 22B, as well as any other two-segment or poly-segment stabilization scenario such as the other embodiments described hereabove. In a preferred embodiment, this universal component includes multiple types of pivoting arms, such as arms that provide different amounts and/or directions of stabilizing forces and or limit ranges of motions in varied distances and orientations.

It should be understood that numerous other configurations of the systems, devices and methods described herein may be employed without departing from the spirit or scope of this application. The pivoting arm of the stabilization device can be attached to bone anchors at its proximal, hinged end, and/or at its translating distal end, with a secured connection that is static (fixed), or it can be secured with a movable, dynamic connection. The pivoting arm and securing connections can be configured to prevent motion of the bone segments, limit motion such as limiting a specific direction or type of motion, or apply specific resistive forces to motion.

The components of the devices of the present invention are preferably configured for percutaneous placement, each device sized for placement through a percutaneous cannula. Each device preferably includes a lumen or sidecar through which a guidewire can be placed, or allowing placement along side a percutaneously placed guidewire. The pivoting arm of the present invention can preferably be rotated, such as with the inclusion of a slot allowing the guidewire to exit a lumen, while a guidewire is in place. The pivoting arm and attached components are preferably configured such that the pivoting arm can be secured, such as with insertion of multiple set screws, also with a guidewire in place. Other components may include slot exits from guidewire lumens such as to allow over-the-wire delivery and subsequently escape the guidewire while leaving the guidewire in place. The devices and methods of the present invention are configured to be inserted without resection of tissue, however procedures including or requiring resection are also supported.

The pivoting arm of the present invention preferably includes one or more functional elements. In a preferred embodiment, an artificial facet or facet portion is included and built into the pivoting arm or other component of the bone stabilization device. Each component may include one or more articulating surfaces, such as one located at the end of the pivoting arm and one on either the receiving assembly or hinged assembly of the present invention, such that pre-defined motion between the two attached bone segments can be achieved.

One difficulty occasionally associated with driving bone screws according to certain embodiments of the present invention is that the pre-assembly of the rod onto the head of the screw eliminates or severely limits the use of current driving mechanisms, as the head of the screw is generally rendered difficult to access or non-accessible.

Certain other embodiments of the invention address this difficulty. It should be noted that such embodiments may in particular refer to assemblies such as element 100 of FIG. 4, but that the same may also be employed in the receiving assembly of element 150.

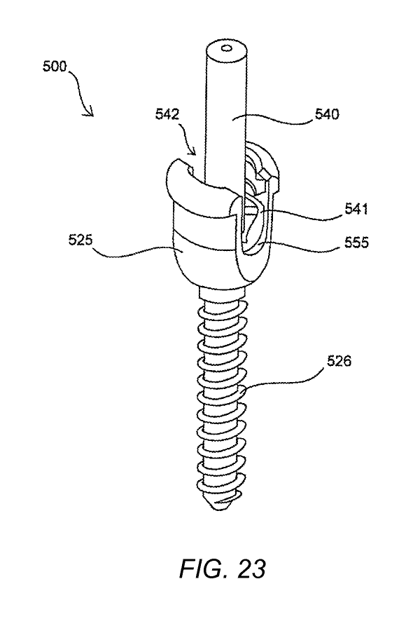

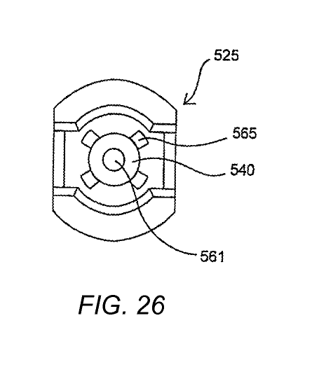

Referring in particular to FIGS. 23-26, a device 500 includes a pivoting arm 540 and a bone anchoring portion including a seat 525. Seat 525 may be a polyaxial seat, such as the seats included in polyaxial pedicle screws commonly used in spine surgery. A lumen 561 (shown in FIG. 24) passes through arm 540 and inside the tube surrounded by screw 526 such that the assembly may be passed, in the orientation shown in FIG. 24, into a patient through a cannula and over a previously-placed guidewire, such as a “K-wire” commonly used in bone and joint procedures.

At the end of arm 540 is ball end 541, which is rotationally received and captured by seat 525. The arm 540 can be inserted into seat 525 by an operator, or may be provided in a pre-attached state. The arm 540 can be removable from seat 525, or may be permanently, though rotatably, attached, whether provided in a “to-be-assembled” or a pre-assembled state. The ball and socket design of FIG. 23 allows multi-directional rotation of pivoting arm 540. Alternative designs may allow a single degree of freedom, or may allow more sophisticated trajectories of travel for the distal end of arm 540. “U”-shaped grooves 542 are provided to allow the rod 540 to be pivoted in a perpendicular (or other angular) fashion relative to screw 526.

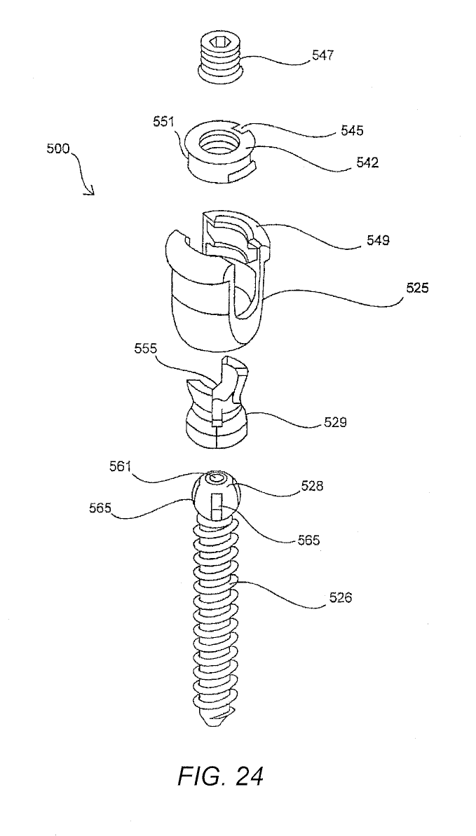

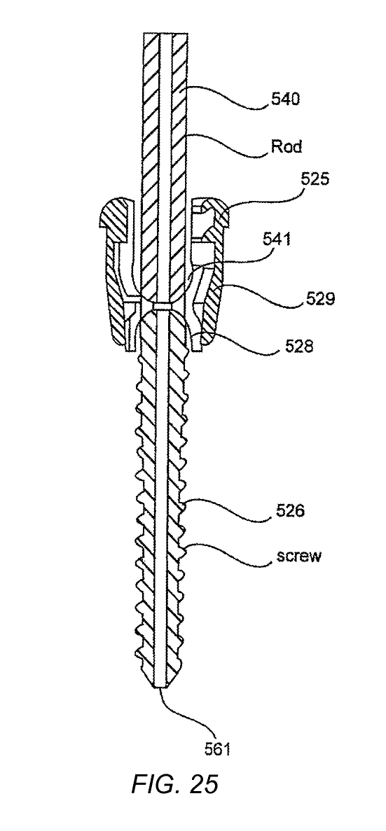

Referring now to FIG. 24, an exploded view of a construction of the bone stabilization device is shown. The system 500 includes screw 526 with screw head 528 which matingly engages with a pivoting element or coupler 529 in, e.g., a ball-and-socket arrangement. The pivoting element 529 engages with the seat 525 via a friction-fit, as seen in FIG. 25. Other ways in which the pivoting element 529 can engage the seat 525 include a snap-fit or other such clearance fit. The pivoting element 529 can also be captured by other means, including a C-ring. In general, any geometric features which can cooperatively engage may be employed, including lugs, recesses, etc. The pivoting element 529 is provided with a hole therethrough to accommodate a guidewire within lumen 561. The pivoting element 529 has two partially-spherical voids formed within, as seen in FIG. 25, to accommodate the base 541 of the rod 540 and the screw head 528.

After the rod has been pivoted to a position for use in a patient, the rod may be held in that position by use of a closure element or cap 542 and a set screw 547. The closure element 542 may be snap-fitted into the seat 525 by interaction of closure element tabs 551 and seat grooves 549. Instead of grooves and tabs, lugs may also be employed. Lugs have the benefit of preventing the seat from splaying and releasing the rod. Furthermore, besides the snap-fit of closure element 542, the same may also be dropped in and captured with set screws or other capture devices. One particular other such capture device includes an integral locking nut/plug combination, which eliminates the need for a plug and set screw set.

A closure element slot 545 may be disposed in the closure element 542 so that the same may be further tightened along the groove 549. Of course, various other techniques may also be used to keep closure element 542 within seat 525. The set screw 547 may then be tightened to secure the rod 540 against movement.

The screws such as screw 526 are generally driven into place in the bone when the rod 540 is in the position shown in FIG. 25, that is, coaxial with respect to the axis of the screw thread. The top of the screw head 528 is then rendered inaccessible, although that is where slots for the driving of such screws are generally disposed. For this reason, at least one peripheral slot 565 may be disposed so that a driver with a cooperating element may be used to rotate the screw 526. As even peripheral slots 565 would be rendered inaccessible by the above-described assembly, one or more corresponding pivoting element slots 555 may be disposed in the pivoting element 529.

In use, the screw 526, the pivoting element 529, the seat 525, the rod 540, and the corresponding intermediate elements, e.g., couplers or rod-capturing elements, are assembled prior to implantation in the patient. The device is inserted over the guidewire. The screw is then driven into the desired bone by use of a driver (not shown) generally having one or more protrusions which are long enough to pass through the seat 525, through intermediate elements, couplers, or rod-capturing elements, and to cooperatively engage with peripheral slots 565. The configuration of the driver protrusions is such that the same can cooperatively engage or mate with corresponding peripheral slots 565. Any number of protrusions and slots may be employed. In certain embodiments, 2, 3, 4, or 5 slots 565 and a corresponding number of protrusions on the driver may be employed. The slots 565 may be equidistantly disposed about the screw head 528 or may be otherwise disposed arbitrarily. Once the screw is driven into the bone, the rod 540 may be pivoted and the closure element 542 and set screw 547 applied.

Further details of the above embodiment may be seen by reference to the previously-described embodiments, in which similar elements have similar descriptions and functions. In particular, over-the-wire drivers may be employed such as described above in connection with FIG. 6.

In some of the embodiments shown in FIGS. 3-22 above, the bone stabilization system was seen to include a first bone anchor with a pivoting rod pre-attached. It should be noted that in some embodiments, the first bone anchor may be inserted without the pivoting arm attached. Once the bone anchor is installed, or at a point during the installation thereof, the pivoting arm may be attached.

Attachment of the pivoting arm may be accomplished using any of the configurations described above. Generally, such attachment is preferably performed in a manner in which minimal force is applied to the bone anchor. One method is to employ a “snap-ring” disposed into the seat to retain the pivoting rod after the same is installed in the seat. In this method, application of the snap-ring into the seat should not put undue or an otherwise significant amount of pressure on the bone anchor.

Various advantages inure to this non-pre-attached pivoting rod embodiment. In particular, the same allows customization of various properties of the assembly, including: length, diameter, curvature, dynamic stabilization performance characteristics, etc., to meet the requirements of the patient's spine.

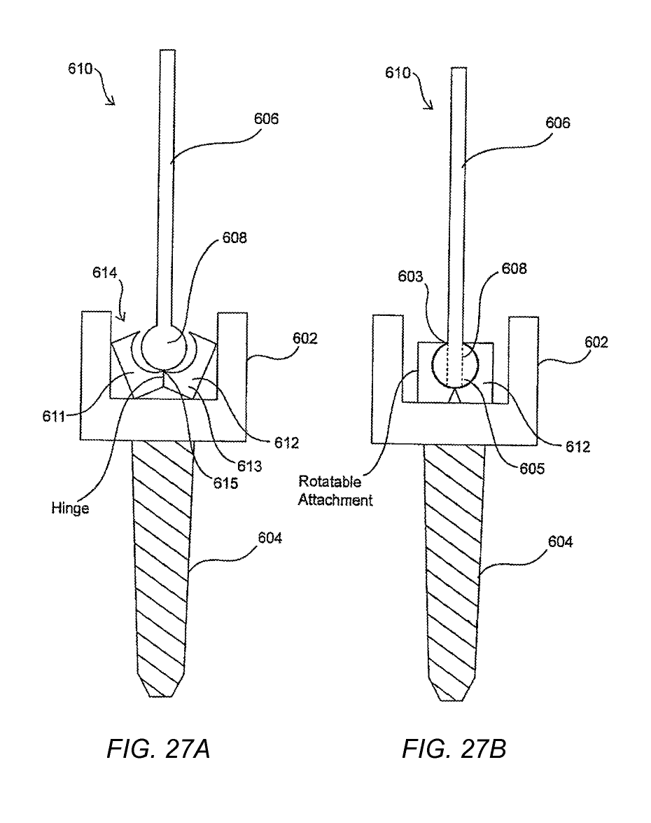

Besides snap-fit or other sorts of frictional attachment mechanisms to connect the pivoting arm to the first bone anchor, a “clam-shell” capture mechanism may also be employed. Referring to FIG. 27, a system 610 is shown with a bone screw 604, a seat 602 having a void 614 formed therein, and a pivoting rod 606 having a distal end 608. Prior to, during, or following installation of the bone screw 604 into the desired bone segment, the distal end 608 is inserted into the void 614 and more particularly into a clam-shell capture mechanism 612. Clam-shell capture mechanism 612 includes a first shell 611, a second shell 613, and a hinge 615 for connecting the first shell 611 and the second shell 613. The first shell 611 and the second shell 613 are coupled to the seat 602 within its void 614.

The shells may be attached to the seat via various means. There may be a cap over the shell. The shell may be slitted to allow expansion for a snap-fit. The shell may also be attached via a friction-fit or hinge, or via a combination of these techniques and devices.

FIG. 27A shows the system during installation of the pivoting rod 606 into the clam-shell capture mechanism 612, and FIG. 27B shows the system following installation. To allow a degree of pivot, the clam-shell capture mechanism 612 may have a varying shape and size of the outlet 603 through which the pivoting rod 606 extends. The overall shape of the interior of the clam-shell capture mechanism 612, when closed, must be such that the pivoting rod 606 is held in a secure fashion. However, the same may be provided with a slit (seen as dotted line 605) through which the rod can pivot. The outlet 603 may also be somewhat larger than the diameter of rod 606 so a degree of movement is provided in the plane of the figure, if desired.

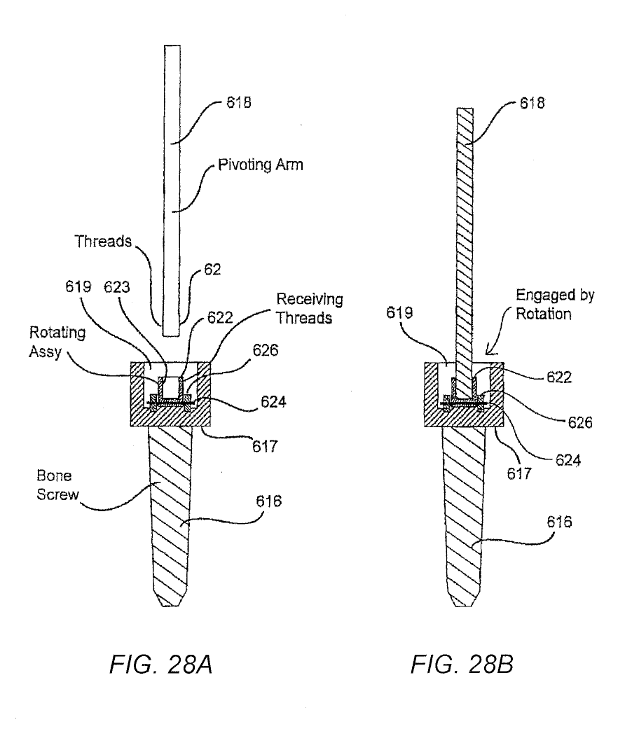

In another system, shown in FIGS. 28A-B, a system is shown with a bone screw 616, a seat 617 having a void 619 formed therein, and a pivoting rod 618 having a threaded distal end 621. Prior to, during, or following installation of the bone screw 616 into the desired bone segment, the threaded distal end 621 is inserted into the void 619 and more particularly into a threaded receiving assembly 622. Threaded receiving assembly 622 includes receiving threads 623, bearings 626, and an axle 624 about which the assembly rotates on the axle. Alternatively, lugs which mate with recesses may be employed. The threaded receiving assembly 622, and in particular bearings 626, are coupled to the seat 617 within its void 619 in known fashion.

FIG. 28A shows the system prior to installation of the pivoting rod 618 into the threaded receiving assembly 622, and FIG. 28B shows the system following installation. Following installation, the pivoting arm 618 may rotate and its distal end captured by a receiving assembly as described above.

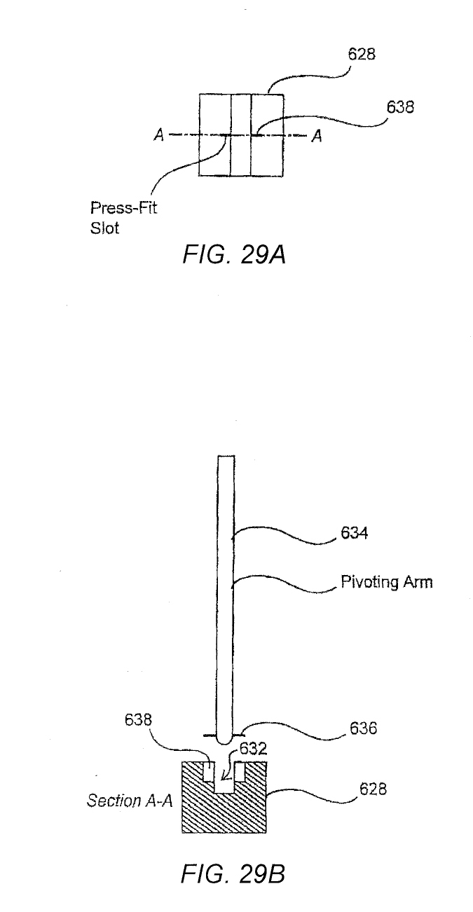

FIGS. 29A-B show top and side views of a frictional-fit engagement for a pivoting rod 634 to attach to a seat 628 of a bone anchor (not shown). Pivoting rod 634 is shown with a small axle 636 therethrough. Of course, axle 636 could also be constituted of two small pins (or one pin which passes all the way through) disposed on opposing sides of the pivoting rod 634. Seat 628 has a void 632 formed therein, with press-fit slots 638 on two sides thereof. Pivoting arm 634, and in particular axle 636, press-fits into the slots 638 and is held in place by the frictional engagement of the axle and the slots. Despite being held in place, the placement of the axle and the slots allows a rotational degree of freedom, in this case out of the plane of the figure. The pivoting arm may then be captured by a receiving assembly as described above.

The slots may have a larger separation opening at the bottom to allow the rod to “snap-in”. In addition, the slots may have a larger separation at the top for ease of insertion. In either case, the slots may be tapered to the larger separation. Both of these tapering may be employed in combination or separately.

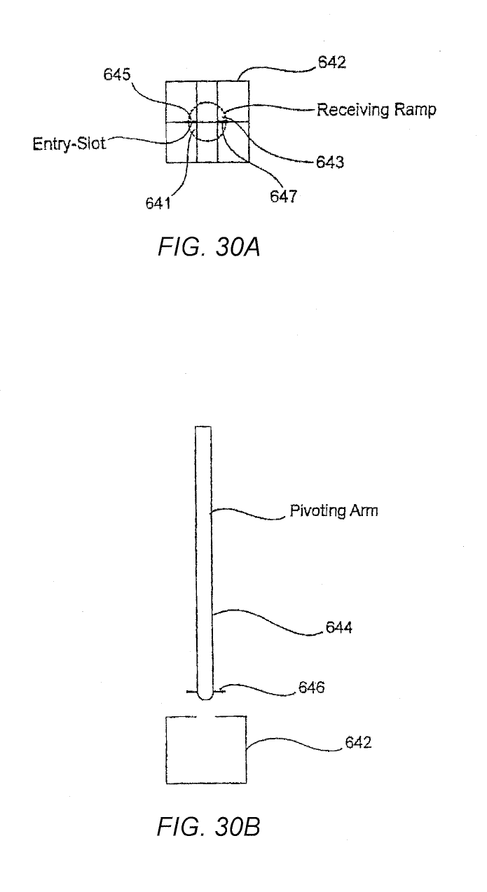

FIGS. 30A-B show top and side views of a related embodiment of a bayonet-fit engagement for a pivoting rod 644 to attach to a seat 642 of a bone anchor (not shown). Pivoting rod 644 is shown with a small axle 646 therethrough, the nature of which is similar to axle 636 above. The seat 642 has two entry slots 645 and 647, which are respectively adjacent receiving ramps 641 and 643. Pivoting arm 644, and in particular axle 646, is disposed in the entry slots 645 and 647 and then twisted to securedly engage the seat 642, in a bayonet-fit fashion. Despite being held in place, the placement of the axle and the slots allows a rotational degree of freedom, in this case out of the plane of the figure. The pivoting arm may then be captured by a receiving assembly as described above (the ramps have a hole in the middle to accommodate rotation of the rod).

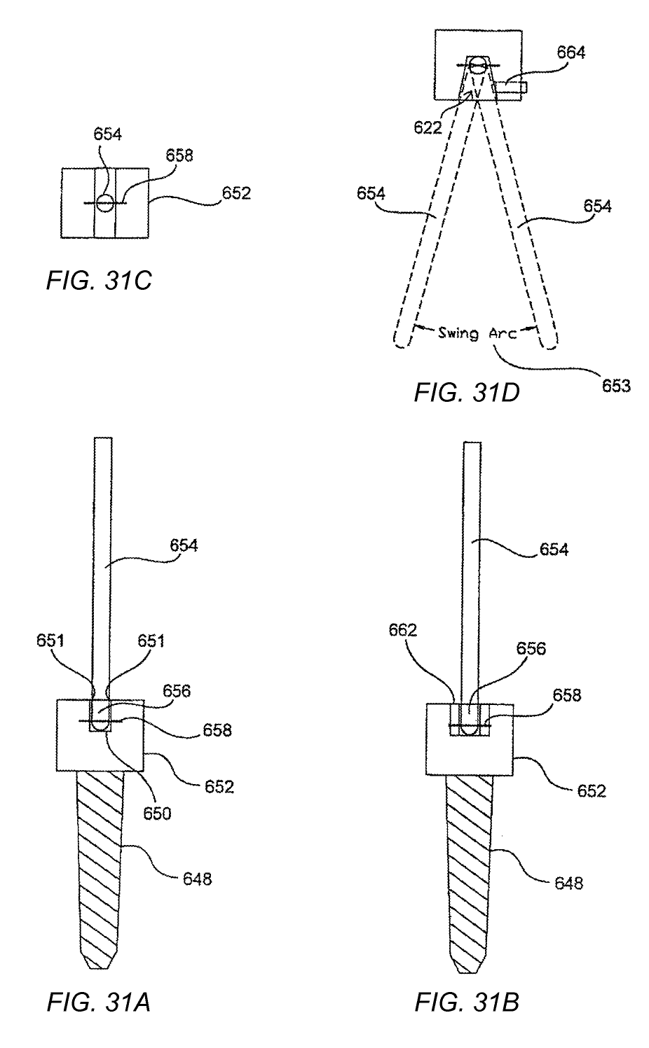

FIGS. 31A-D show assemblies for frictional-fit engagements for a pivoting rod to attach to a seat of a bone anchor, where the degree of range of motion is controllably adjusted. The degree of range of motion may be in travel, angle, or other sort of motion.

In particular, referring to FIG. 31A, pivoting rod 654 is shown with a small axle 658 through a distal end 656 thereof. In a manner similar to that of FIGS. 29 and 30, the pivoting rod is securedly attached to a seat 652, within a groove 650, which in turn is attached to bone screw 648. The side walls 651 of groove 650 may be closely fit to the distal end 656 of the pivoting rod 654 or they may be spaced more apart. If they are closely-fit, as shown in FIGS. 31A-C, then the swing of pivoting rod 654 is substantially limited to a single plane. On the other hand, if the side walls 651 of groove 650 are spaced apart to form a void 662 in which sits the distal end 656 of the pivoting rod 654, as shown in FIGS. 31B and D, then the swing of pivoting rod 654 has considerably more movement or motion. In this case, the swing of pivoting rod 654 is defined by an arc 653. A set-screw 664 may be disposed to control the size of arc 653. Note that the void 662 may be generally trapezoidal in shape, and that the size of the slots in which the axle 658 is disposed may also be somewhat enlarged to accommodate movements of the axle and rod.

Further, while production of an arc-allowed movement for a pivoting rod is shown, analogous alterations in the side walls and axles and slots would allow additional movements such as: flexion, extension, axial rotation, lateral bending, etc.

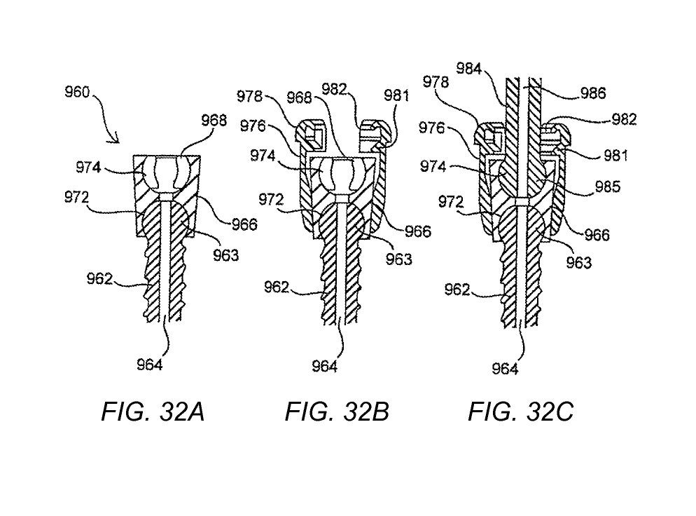

Referring ahead to FIGS. 32A-C, another way of frictionally engaging a pivoting rod to a seat of a bone anchor is shown, as well as a way of frictionally engaging a seat to a bone anchor.

Referring to FIG. 32A, a system 960 is shown where a bone screw 962 has a guide lumen 964. Following, during, or before installation of the bone screw 962, a snap-in tapered screw retainer 966 is attached to the bone screw 962, in particular by frictionally engaging the screw head 963 to a first screw void 972 formed in screw retainer 966. In one embodiment, slots (not shown) may be formed in the screw retainer 966 around first screw void 972 in order to allow a portion of the screw retainer 966 to “flare” outwards to accept and frictionally engage the screw head 963. A second screw void 974 is formed in the screw retainer 966 generally opposite the first void. The second screw void 974 is configured to accept a pivoting rod following, during, or before installation of the bone screw 962. The second screw void 974 includes an elastic member 968 to assist the securing of the pivoting rod.

Following installation of the screw head 963 into the screw retainer 966, the screw retainer 966 is inserted into a seat 976. Seat 976 includes two lips, lip 981 for securing the screw retainer and lip 982 for securing the pivoting rod. The top end of the screw retainer 966, due to its inherent elasticity, compresses somewhat as it passes lip 981. Following insertion, the top end springs back to its original configuration. The screw retainer 966 outer diameter is greater than the inner diameter of the seat 976, preventing the screw retainer from coming out of the seat. Moreover, a force pulling the screw downward would likewise cause the first void to tighten around the screw head because the first void would itself be caused to decrease in radius due to the inner diameter of the seat. In other words, a force pulling the screw downward also prevents the screw from coming out because any such force pulls the capturing element in such a way as to make the capturing element tighten around the head of the screw, preventing removal.

Once the seat is installed, the pivoting rod 984 with guide lumen 986 and ball end 985 can then be snap-fit into the second void 974. A clearance or space is provided adjacent the second void such that the same can flare out and securely accept the rod.

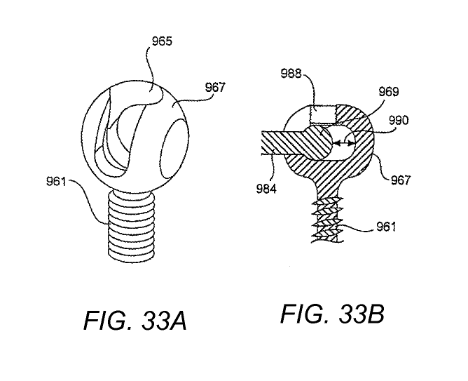

FIGS. 33A-B show an alternative embodiment of a rod and bone anchor assembly. In particular, referring to FIG. 33A, a bone screw 961 is shown with a seat 967 having a void 965 therein. Referring to FIG. 33B, a pivoting rod 984 with ball end 969 has been disposed into the void 965 of the seat 967. A plug 988, which may have threads that engage corresponding threads on the opening of the void, is used to secure the pivoting rod in place. The rod is disposed such that a space 990 is left within void 965 which allows the rod to slide back and forth once the rod is rotated into position, approximately at a 90 degree angle with the screw 961.

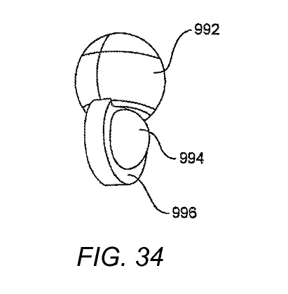

FIG. 34 shows a device that may be employed in the above embodiments of a rod and bone anchor assembly. In particular, a connector 991 is shown having a tip 992 for capturing a rod (not shown) or a screw retainer which then in turn connects to a rod (not shown). Connector 991 also has a tip 994 having ridge 996 that connects to a bone screw. The ridge 996 allows a rotational force to be transmitted through to the bone screw if desired.

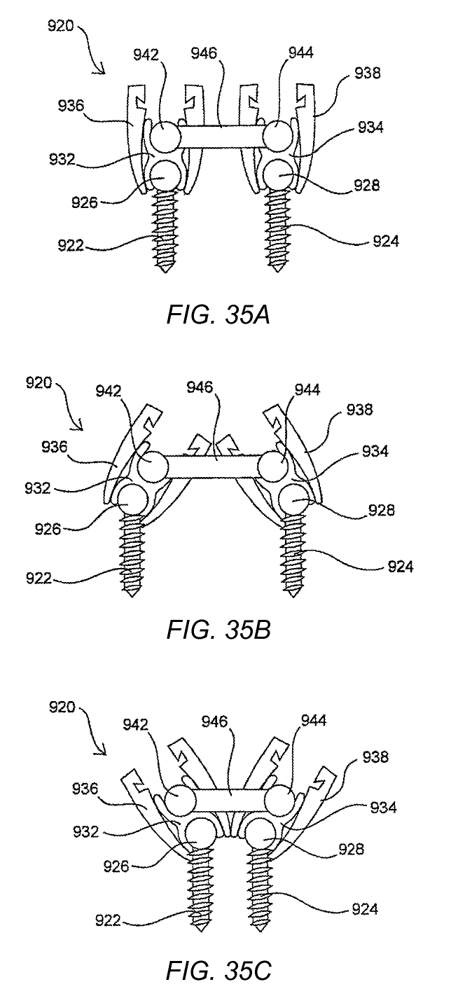

Systems according to the invention may also include those that can provide a degree of flexibility to allow a more convenient capture of a pivoting rod. Referring to FIGS. 35A-C, a system 920 includes two bone screws 922 and 924 that are shown with respective screw heads 926 and 928. Each screw head is disposed in a first void formed in respective retaining members 932 and 934. Retaining members or seats 932 and 934 each have a second void formed therein substantially opposite the first void. The second void contains the ball-shaped ends 942 and 944 of rod 946. Seats 936 and 938 contain respective retaining members 932 and 934. Seats 932 and 934 perform functions similar to those shown in FIG. 32.

The ability of the retaining members or seats to pivot and rotate about the screw head allows the retaining members or seats to be disposed in a number of different positions relative to the axis of the screws. This is important as the screw axes are generally non-parallel as the same depends on the orientation of the pedicle in which they are installed. The retaining members or seats can thus be oriented arbitrarily and independently, and can in particular be oriented such that the pivoting rod can be conveniently installed. In so orienting the retaining members or seats, a degree of compression or distraction is often imparted to the spinal segments.

In an actual installation, typically the rod would be disposed between the retaining members or seats, and a set screw would be started in each to retain the rod. Then a degree of distraction or compression would be imparted to better seat the rod, and the set screw would then be tightened. In this way, the set screw is always properly placed in the retaining members.

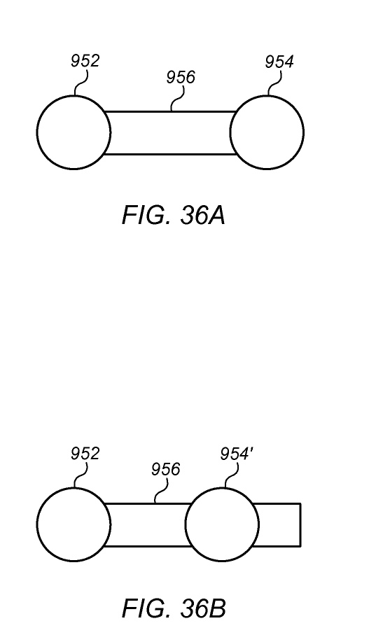

FIGS. 36A-B show an alternative embodiment 950 of a rod 956 that may be employed in the system of FIG. 35. Rod 956 has a stationary ball end 952 and a movable ball end 954. Movable ball end 954 can slide back-and-forth along rod 956. The same can be secured by methods and devices described here, including set screws, friction-fits, crimping, etc. As the ball end 954 must still be disposed in the void within retaining member 934 (which in turn sits within seat 938), retaining member 934 and seat 938 may be configured with a slot substantially opposite to the slot facing seat 936. This slot, opposite to the slot facing seat 936, allows an excess rod portion 955 to exit the retaining member 934 and seat 938 in the case where the ball end 954 is not at the extremity of the rod 956.

It should be noted with respect to this embodiment that the ball end 954 may be deployed such that it can slide easily along rod 956, or can slide with effort along rod 956, or cannot slide along rod 956. Moreover, a universal joint-type end may be situated at either ball end, or may also be disposed at an intermediate position along rod 956.

While numerous varieties of pivoting rod have been disclosed above, even more types may also be employed. For example, a locking cone system, as shown in FIG. 18 above, may allow a single device to accommodate a continuous range of sizes of pivoting rods.

Further, while numerous varieties of capture and receiving assemblies have been disclosed above, even more types may also be employed. For example, the pivoting rod may be swaged into place or otherwise captured. In any case, the initial attachment of the pivoting rod to the initial seat may be permanent or detachable. Moreover, the secondary attachment of the pivoting rod to the capture seat or other receiving assembly may also be permanent or detachable. Following rotation of the pivoting rod, the same may be fixed in place with, e.g., set screws or other means.

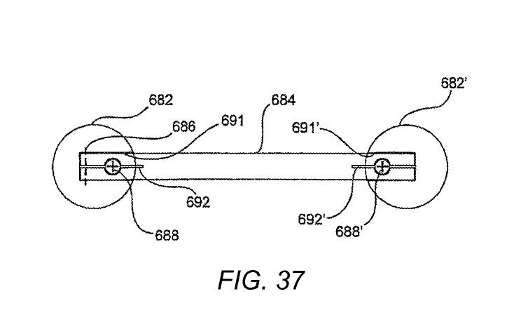

As another example, referring to FIG. 37, a system is shown with a pivoting rod 684 which pivots about axle 686 such that the pivoting rod 684 extends from a seat 682 to a seat 682′. Slots 692 and 692′ are provided in the pivoting rod 684 at extremities thereof. A screw 688 is disposed which intersects slot 692, and correspondingly a screw 688′ is disposed which intersects slot 692′. When the pivoting rod 684 is in a deployed configuration, as shown, screws 688 and 688′ may be tightened, which in turn widens slots 692 and 692′ respectively. As the slots widen, the extremities of rod 684 bow outward and are forced against sidewalls 691 and 691′, frictionally engaging the same. Once the frictional engagement is great enough, pivoting rod 684 is secured between the seats, and bone stabilization occurs. Again, it is noted that the screws 688 and 688′ need not provide a force normal to the plane of the figure, frictionally securing the rod against the seat. Rather, the screws bow the rod ends outward, parallel to the plane of the figure, frictionally securing the rod against the sidewalls.

Of course, a set screw may also be used that does provide a force normal to the plane of the figure, frictionally securing the rod against the seat.

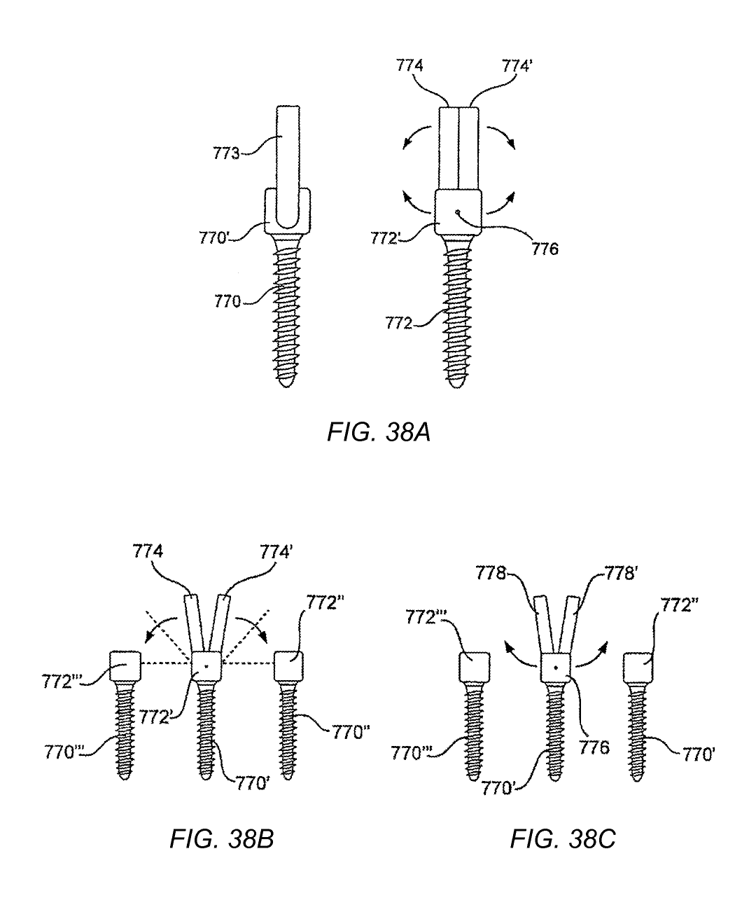

As noted above in connection with the discussion corresponding to FIGS. 10-13, 16, 19, and 22, embodiments of the invention may not only be used to provide stabilization to two adjacent vertebrae, but indeed can be used in a multi-level fashion to stabilization three or more vertebrae. Additional details concerning these designs may be seen by reference to FIGS. 38-43.

Referring to FIGS. 38A-C, a system is shown in which two bone screws 770 and 772 are shown, each with an associated respective seat 770′ and 772′. Seat 770′ houses one pivoting rod 773, while seat 772′ houses dual pivoting rods 774 and 774′. Seat 772′ with dual pivoting rods further has an axle 776 about which each rod pivots. Rod 773 also has an axle (not shown). The dual rod system can be loaded into the seat at any time, before, during, or after installation of the bone anchor, to allow connection to adjacent screws, e.g. at seat 770′.

Referring to FIG. 38B, a system is shown in which the dual-rod system of FIG. 38A (right hand side) is shown between two bone anchors. These two bone anchors are not shown with their own rods, but the same may also be incorporated. To the right of bone anchor 770′ and seat 772′ is bone anchor 770″ and seat 772″. To the left of bone anchor 770′ and seat 772′ is bone anchor 770″ and seat 772″. In FIG. 38B, the dual rod system is connected to the seat at their distal end, in which case the rods rotate down to be captured by receiving assemblies, one rotating clockwise and the other counter-clockwise.

Referring to FIG. 38C, a system is shown in which a related dual-rod system is shown between two bone anchors. As before, these two bone anchors are not shown with their own rods, but the same may also be incorporated. The dual-rod system has a bone anchor 770′, seat 776, and two rods 778 and 778′. To the right of bone anchor 770′ and seat 776 is bone anchor 770″ and seat 772″. To the left of bone anchor 770′ and seat 772′ is bone anchor 770″ and seat 772″. In FIG. 38C, the dual rod system is configured such that the rods slide outward, from their distal ends, such that the distal ends then become the portions captured by receiving assemblies.

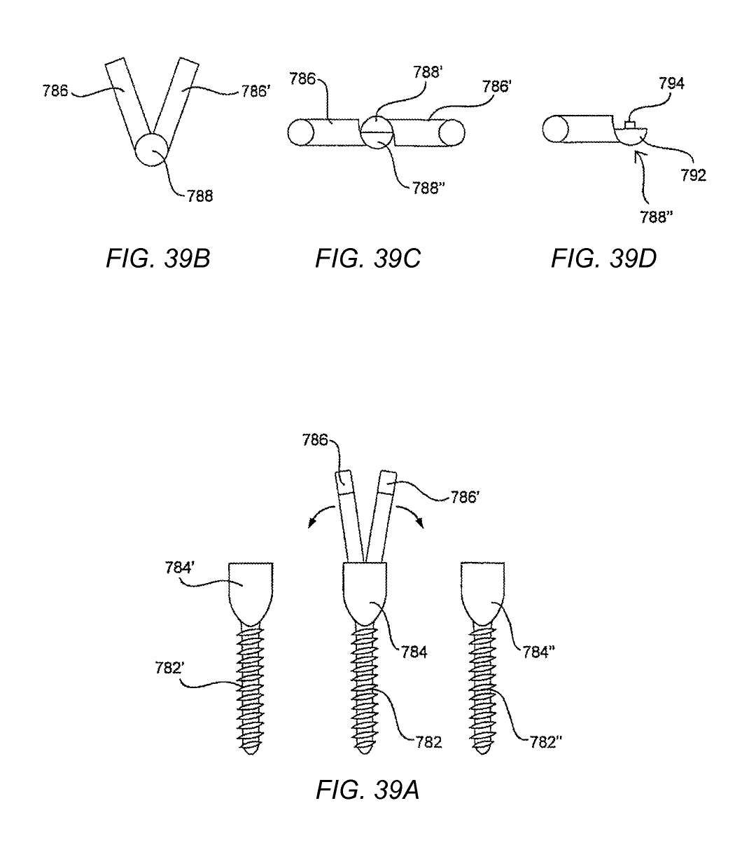

FIGS. 39A-D show an embodiment related to that of FIGS. 38A-C. In particular, referring to FIG. 39A, a bone screw 782 is shown with a seat 784 and a dual-rod assembly having rods 786 and 786′. On the left side of bone screw 782 is a bone screw 782′ with a seat 784′, and on the right side of bone screw 782 is a bone screw 782″ with a seat 784″. Rod 786′ rotates in a clockwise direction to engage a capture mechanism (not shown) within seat 784″, and rod 786 rotates in a counter-clockwise direction to engage a capture mechanism (not shown) within seat 784′.

FIG. 39B shows additional details. In particular, the figure shows a rotation mechanism 788 through which rods 786 and 786′ rotate. In particular, referring to FIG. 39C, rotation mechanism 788 has a first half 788′ and a second half 788″. First half 788′ and second half 788″ matingly engage, e.g., each can form half of a sphere, and the two combined can approximately form a complete sphere. FIG. 39D shows a plug 794 formed on an interior wall of half-sphere 792 of second half 788″ which can matingly engage a corresponding hole (not shown) in 788′. Other rotation mechanisms can also be employed.

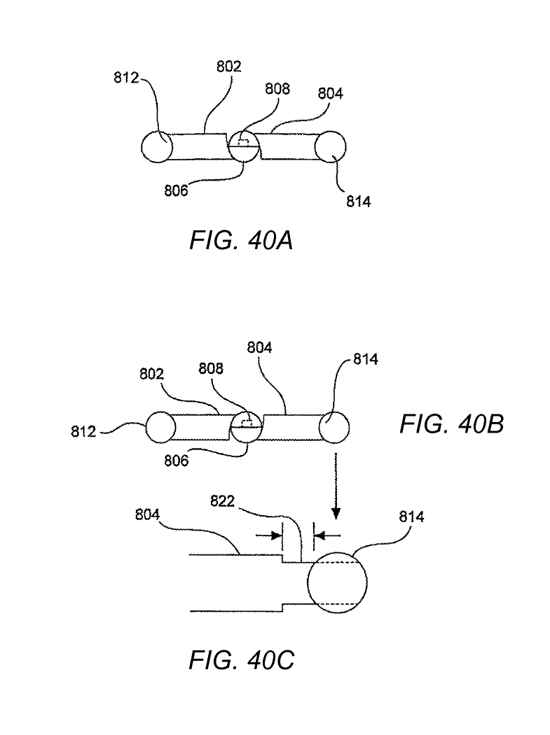

Other systems can also provide multilevel stabilization. FIGS. 40-44 show additional embodiments of systems employing dual arms on a single hinged assembly.

In particular, FIGS. 40A-C show a dual arm system with a unitary hinged assembly employing adjustable-length rods. In this embodiment, pivoting rods 802 and 804 meet at a rotation mechanism having first half 806 and second half 808. The rotation mechanism may be like that disclosed above. The rotation mechanism snaps into place in a seat like those disclosed above. A first ball 812 is disposed at an end of rod 802 opposite that of first half 806, and a second ball 814 is disposed at an end of rod 804 opposite that of second half 808.

In some of the above-described capture mechanisms, a pivoting rod is that which is captured, and the same is secured by a threaded plug, set screw, or other such retainer. Accordingly, the system is per se adjustable because the rod may be captured at any point along its length. In FIGS. 40A-C, if the ball is that which is to be captured, then the length of the rod becomes much more important. Accordingly, in FIGS. 40A-C, the ball 814 is attached to an inner rod 822 (see FIG. 40C) which is slidably and telescopically disposed within rod 804. Inner rod 822 may become immovable with respect to rod 804 in a number of ways, including via use of a set screw, by rotation of inner rod 822 on which a cam is biased to engage the inner wall of rod 804, etc. Alternatively, the same may be left to slidably move relative to rod 804, depending on the desires of the physician.

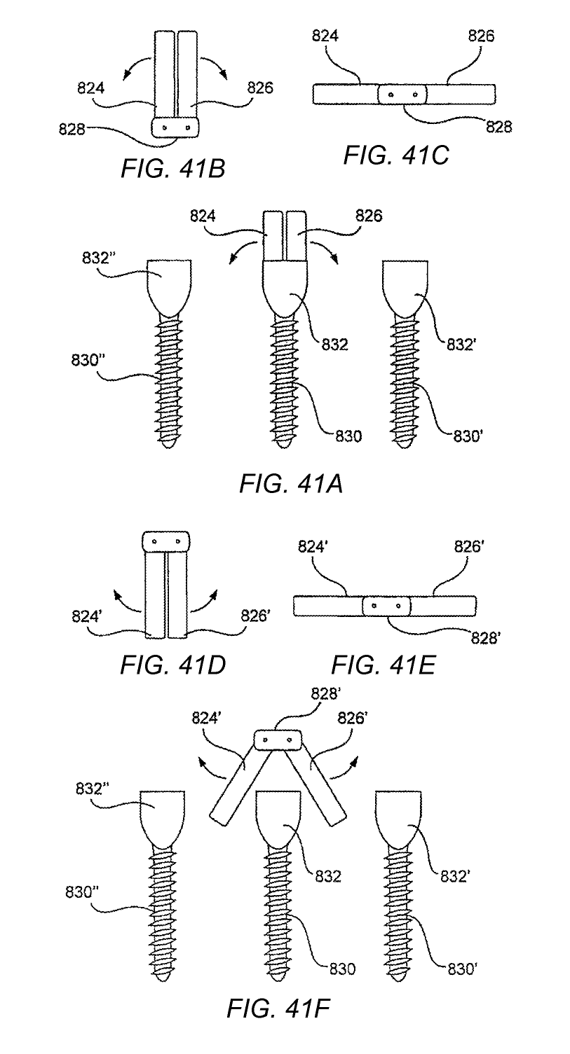

FIGS. 41A-F show a dual arm system with a unitary hinged assembly employing multiple axles for the pivoting rods. Referring to FIGS. 41A-F, a bone screw 830 is shown with a seat 832 and a dual-rod assembly having rods 824 and 826. On the left side of bone screw 830 is a bone screw 830″ with a seat 832″, and on the right side of bone screw 830 is a bone screw 830′ with a seat 832′. Rod 826 rotates in a clockwise direction to engage a capture mechanism (not shown) within seat 832′, and rod 824 rotates in a counter-clockwise direction to engage a capture mechanism (not shown) within seat 832″.

FIG. 41B shows additional details. In particular, the figure shows a rotation mechanism 828 through which rods 824 and 826 rotate. In particular, the rotation mechanism includes dual parallel axles, each attached to one of rods 824 and 826.

FIG. 41B shows the rods in a parallel alignment, such as during insertion. FIG. 41C shows the rods in an anti-parallel alignment, such as following deployment.

FIG. 41F shows the same set of bone screws and seats, this time being engaged by pivoting rods 824′ and 826′ which are coupled together via rotation mechanism 828′. In this embodiment, the step of pushing the rod assembly down acts to automatically open the rods, swinging the same into position where they may be captured by an appropriate receiving assembly. In a manner similar to that of FIGS. 41B-C, FIG. 41D shows the rods in a parallel alignment, such as during insertion, while FIG. 41E shows the rods in an anti-parallel alignment, such as following deployment.

In all of these embodiments, it should be noted that the rod can be pre-attached to the seat or alternatively the same can be installed in the seat following installation of the bone screws into the spine of the patient.

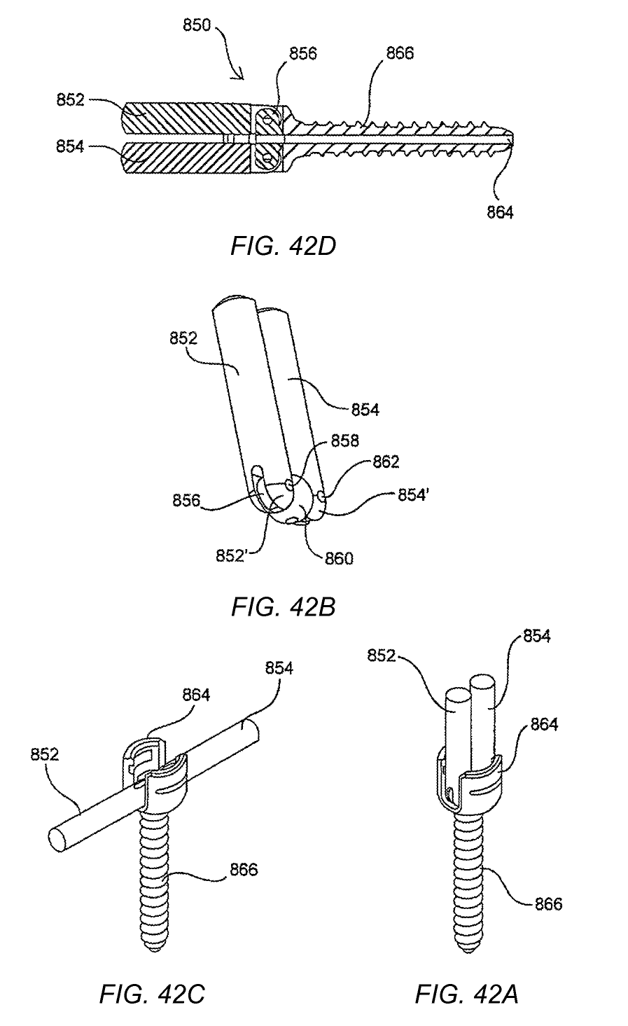

FIGS. 42A-D show an alternative dual arm system 850 with a unitary hinged assembly employing multiple axles for the pivoting rods. In particular, rods 852 and 854 are shown with distal ends 852′ and 854′ (see FIG. 42C), respectively. These distal ends each have a groove into which a flat extension 856 is disposed. Flat extension 856 (and a corresponding flat extension (not shown) within rod 854 are attached to central assembly 860. Moreover, through the flat extensions axles 858 and 862 are disposed, which extend from one side of the distal ends 852′ and 854′ to a side diametrically opposite. In this way, rods 852 and 854 are hingedly attached to central assembly 860.

The distal ends of the rods are disposed within a seat 864 attached to a bone screw 866 having a guidewire lumen 864 disposed therein.

FIG. 42A shows the rods in a position for insertion and FIG. 42B shows the rods in a deployed configuration.

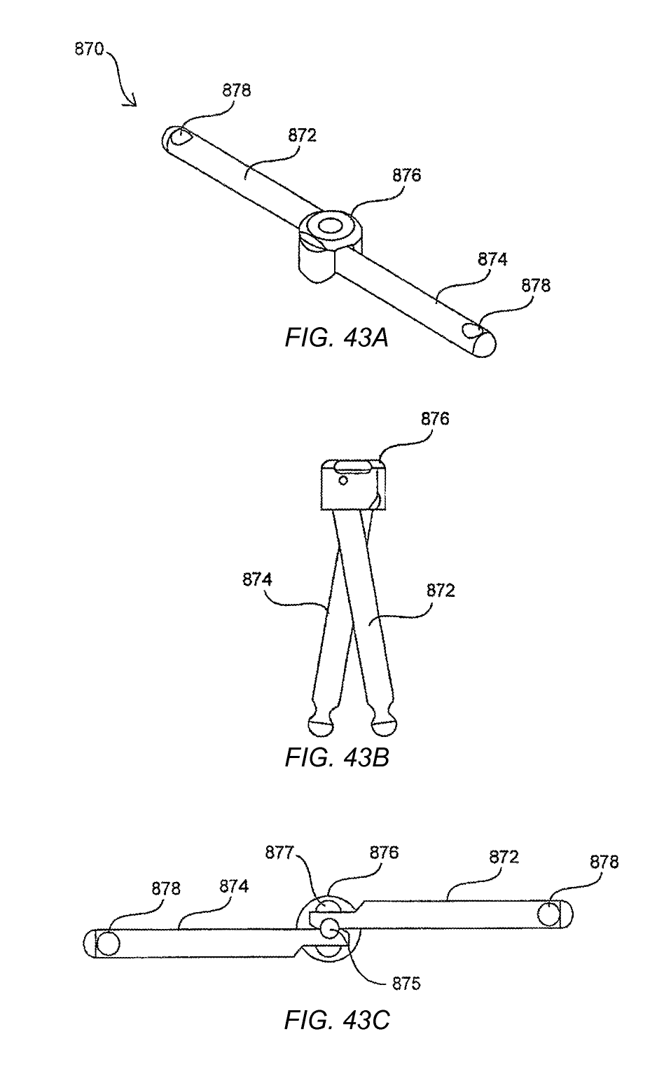

FIGS. 43A-C show a dual arm system 870 with a unitary hinged assembly employing pivoting offset rods. In particular, rods 872 and 874 are shown with distal ends having indentation features 878. Indentation features 878 allow for secure connection to other seats on a multilevel system.

Rods 872 and 874 are joined at a rotation mechanism 876 which includes an axle 877 about which both rods rotate. Multiple axles may also be employed. When the rods are in an insertion configuration, they are generally parallel to each other. When the rods are deployed, they are anti-parallel to each other. A guide lumen 875 may be employed for placement.

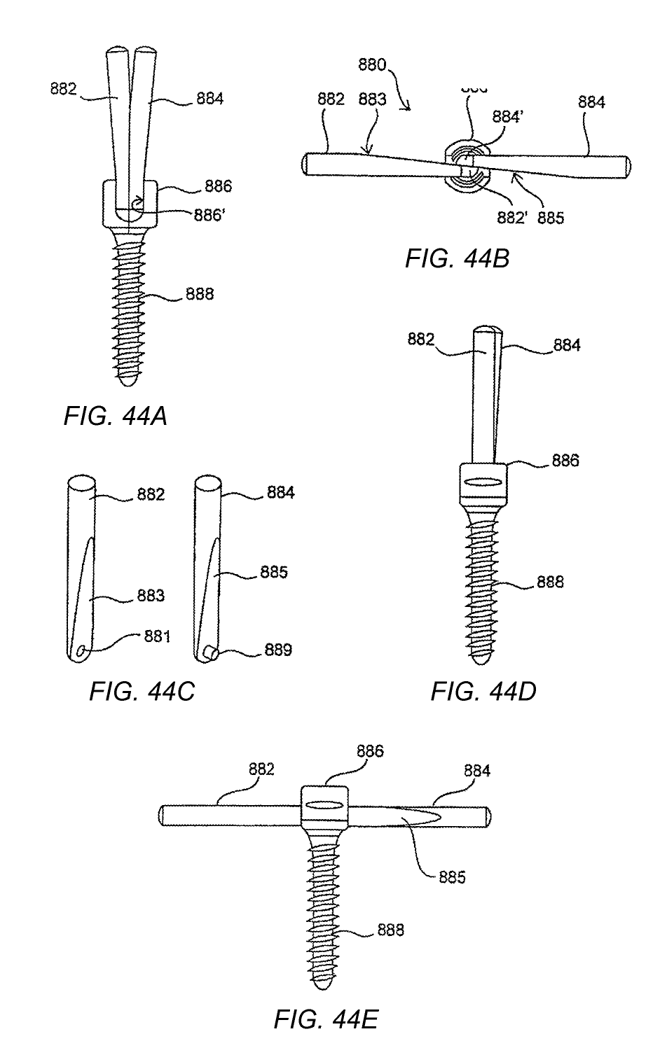

FIGS. 44A-E show a dual arm system 880 with a unitary hinged assembly employing pivoting rods, each with a complementary taper. In particular, rods 882 and 884 are shown joined within seat 886 attached to bone screw 888. The rods may rotate relative to each other via an axle or other mechanism (not shown). For example, referring to FIG. 44C, the rod 884 may have a plug 889 formed on a end 882′ which matingly engages a hole 881 formed on an end 884′ of rod 882. When the plug 889 engages the hole 881, the ends 882′ and 884′ of rods 882 and 884 adjacent the plug and hole form a substantially spherical head which may be securely and rotatably inserted within seat 886. A slot 886′ may be formed within the seat 886 into which the rods rotate when deployed. To allow the rods to align in a substantially parallel manner during, e.g., insertion, each rod may be formed with a cooperating taper. In the figures, rod 882 is formed with a taper 883 and rod 884 is formed with a taper 885. The tapers are formed in a manner such that the face each other when the rods are disposed in the seat, either before, during, or after installation of the bone screw.

When the rods are in an insertion configuration, they are generally parallel to each other, as shown in FIGS. 44A-D. When the rods are deployed, they are generally anti-parallel to each other, as shown in FIG. 44E. Of course, they are still deployed through the cannula.

Other multi-level systems have been disclosed above, in particular, dual attaching cradles on a single receiving assembly are shown in FIGS. 12 and 13, and a sequential arrangement, having a hinged assembly and an attaching cradle coupled to a bone anchor, is shown in FIG. 22.

Many of the dual arms disclosed above show two arms attached to a single seat on a bone screw, i.e., dual pivoting rods on a unitary hinged assembly, these rods then linking to two receiving assemblies diametrically opposed from each other. However, it is noted that a receiving assembly itself may also include a rotatably attachable pivoting rod. In this case, clearance should be allowed for the rotation, typically via a ball-and-socket or hinge, while still allowing secure attachment of the first pivoting rod. One way of configuring this is for each bone anchor to include a receiving assembly (for a first pivoting rod) and a separate seat for attachment of a second pivoting rod (which is then received by another receiving assembly). An advantage of this configuration is that the bone screw/seat/pivoting rod/receiving assembly systems can all have the same or a similar construction, easing manufacture. There is no need to have a separate construction for the hinged assembly vis-a-viz the receiving assembly. Such an embodiment is shown above in FIG. 22B with particular reference to assemblies 70a and 70b.

The above description has disclosed devices and methods for minimally-invasive surgery. Certain additional complementary features may apply to many or all of the above.

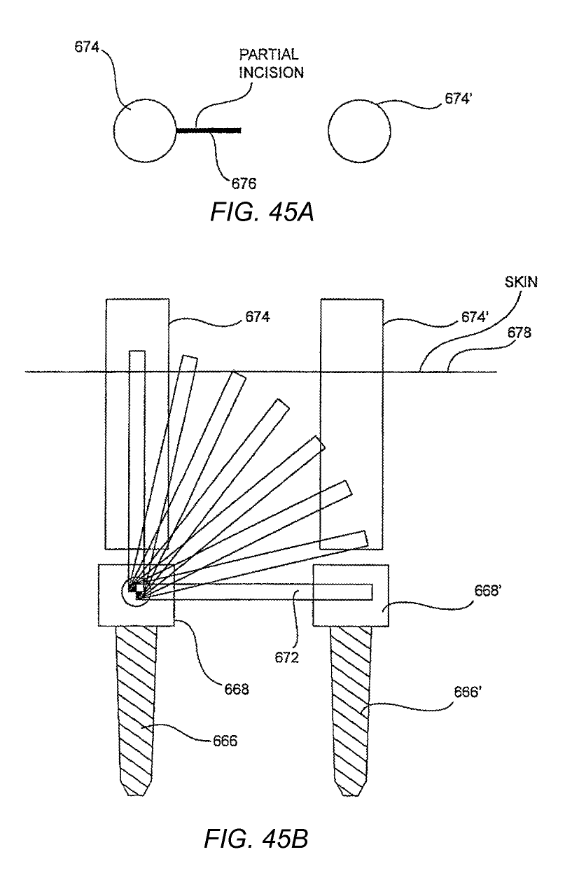

For example, referring to FIGS. 45A-B, two bone screws 666 and 666′ are shown below skin 678. Seats 668 and 668′ are attached, or integral with, respectively, bone screws 666 and 666′. A pivoting rod 672 has a proximal end attached to seat 668 and when deployed extends to and is captured by seat 668′. Insertion cannulae 674 and 674′ are shown above their respective seats and bone screws. As may be seen, when in the insertion configuration, and due to the length of the pivoting rod 672, pivoting rod 672 extends a distance above skin 678. A shorter pivoting rod would not extend above the skin, and could be immediately rotated into the receiving assembly. However, due to the length, the pivoting rod cannot be rotated into seat 668′. In this case, a partial incision 676 may be made to accommodate a partial amount of the rotation of the pivoting rod 672. The first part of the rotation of the pivoting rod passes through the skin 678 through the partial incision 676. In this way, the partial incision 676 allows use of a longer pivoting rod, as may be desired for certain procedures. The same may also accommodate sites that are located closer to the skin.

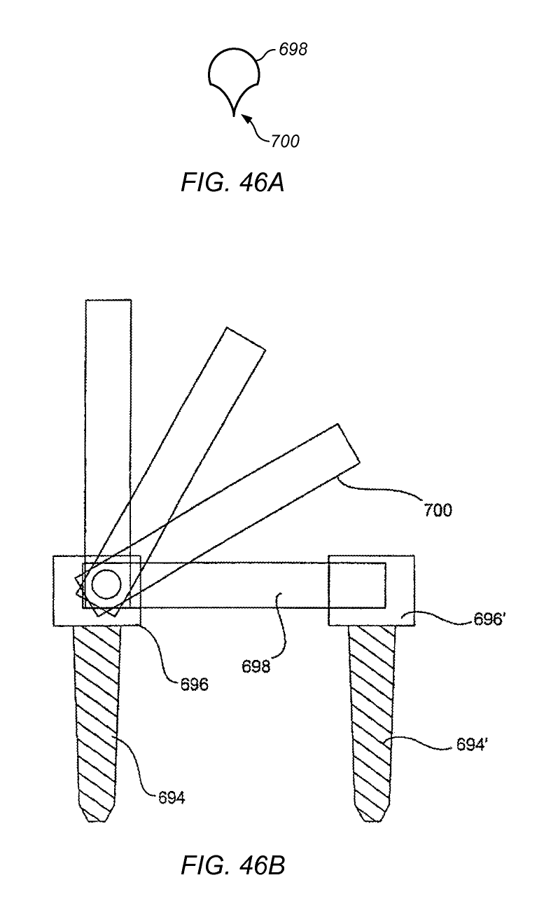

Systems may also be employed that nearly-automatically perform a level of dissection per se. Referring to FIGS. 46A-B, a system is seen with two bone screws 694 and 694′, respective seats 696 and 696′, and pivoting rod 698. The pivoting rod 698 is constructed with an anterior facing edge 700 that is sharpened to reduce the forces required to pass through tissue during the rotation of the pivoting rod 698 into the receiving assembly such as seat 696′. In other words, during rotation, sharpened edge 700 can improve dissection to allow passage of the pivoting rod 698 through the skin and surrounding tissues.

In an alternative embodiment to FIGS. 46A-B, sharpened edge 700 may be blunted prior to the closing procedure. Alternatively, the sharpened edge itself, though not the pivoting rod, may be made biodegradable such that, over time, it would dissolve in the body. The sharpened edge could also be filed off or otherwise dulled by the physician, or a collar may be slid onto the edge so that the sharpened edge is not unsheathed while maintained in the body.

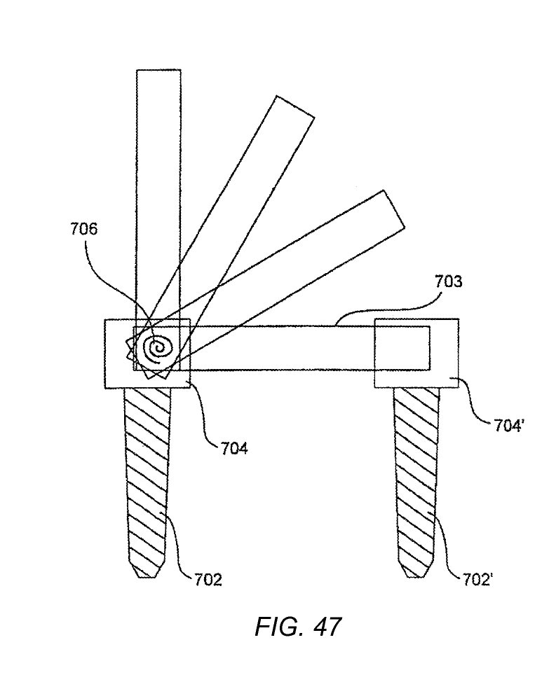

To assist in insertion and installation or in maintenance in a deployed position, the pivoting rod can be combined with a torsional spring to bias the pivoting arm in various positions. Referring to FIG. 47, a system is seen with two bone screws 702 and 702′, respective seats 704 and 704′, and a pivoting rod 703. The end of pivoting rod 703 that is initially disposed within a seat, i.e., seat 704, is also coupled to a torsional spring 706. The torsional spring 706 may resiliently bias the pivoting rod 703 in a position parallel to bone screw 702, perpendicular to the axis of the bone screw 702, or at any angle in between as may be desired.

In the case where the torsional spring 706 resiliently biases the pivoting rod 703 in a position perpendicular to bone screw 702, the rotation procedure may be simplified as the pivoting rod will naturally move to the “captured” or “received” configuration. In the case where the torsional spring 706 resiliently biases the pivoting rod 703 in a position parallel to bone screw 702, the insertion procedure may be simplified as the pivoting rod will move more easily down the cannula. The parallel position will also result in a more convenient removal or readjustment following the pivoting action, if necessary or desired. The angular position of torsional spring 706 may be reset at any time to change the bias, i.e., the “rest” position. This bias may be adjustable by the physician. For example, the spring may be attached to the seat with a screw such that rotation of the screw alters the rest position of the spring.

Of course, the torsional spring 706 may be biased at any point between the two extremes discussed above, and many different functional elements may be employed to resiliently bias the spring in one or more positions. For example, different types of springs or other elastic members may be employed.

Other systems which may maintain a pivoting rod in one configuration or another are shown above. In particular, the above-described FIGS. 31A-D show a system in which the frictional engagement between the rod 654 and the groove walls 651 allow a degree of maintenance of the rod in a desired position. In other words, if the groove walls 651 fit the rod 654 tightly, the same is resiliently held in a given position. This embodiment has an advantage that the any position may be the “resiliently-biased” position, as placement of the rod in any rotational position naturally becomes the “rest” position (or which may be set by the physician via an adjustment), and any movement out of that position is met with a return force, unless and until the movement out of that position becomes so great that a new “rest” position is attained. This embodiment also has the advantage that the rod is secured against small movements, as may occur if the connection between the seats is not tight.

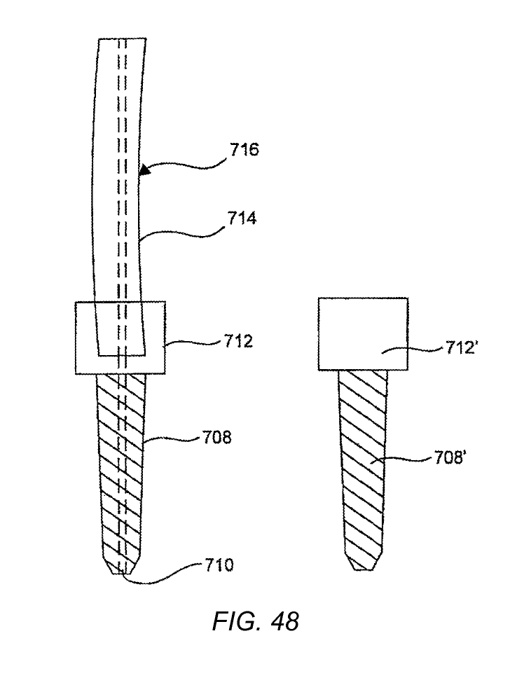

The pivoting rod may be curved or otherwise contoured to approximately mimic the curvature of the spine. Referring to FIG. 48, a system is seen with two bone screws 708 and 708′, respective seats 712 and 712′, and a pivoting rod 714. The pivoting rod 714 has a curved shape 716, which somewhat matches the curve of the spine. However, a guidewire lumen 710 may be provided that is maintained straight throughout the bone screw 708, the seat 712, and the pivoting rod 714. The straightness of the guidewire lumen 710 allows use of even a relatively stiff K-wire. The guidewire lumen can form a slot, open on one side, rather than a hole, so that the guidewire can be left in place even during rotation of the rod into the capture or receiving assembly.

In a related embodiment, the guidewire lumen may also be curved, but may be curved such that the same has a larger radius of curvature than the radius of curvature of the rod. That is, the guidewire lumen is straighter than the rod. In this way, a guidewire may more easily pass through, i.e., with less bending. In another related embodiment, the guidewire lumen may have a greater inner diameter than usual, i.e., much larger than the guidewire diameter, and again this would result in minimized bending of the guidewire as the same passes through.

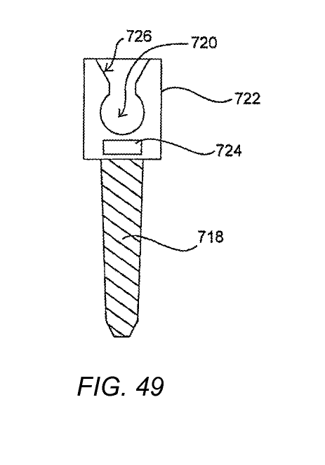

Embodiments may include assistance or confirmation of proper engagement with the receiving assembly or attaching cradle. Referring to FIG. 49, a system is shown with a bone screw 718 capped by a seat 722. This system has a flared opening 726 leading to a capture void 720 that receives the pivoting rod (not shown). The taper of the flared opening 726 provides a snap-fit for the pivoting rod that in turns lead to audible and/or tactile feedback for the physician. An optional magnet 724 may also be employed to assist in the alignment of the rod, which would include a magnetic element in this embodiment. The flared opening further has the advantage of serving to self-align the pivoting rod as the same is guided into place.

In this embodiment the magnetic material may either be a separate piece attached to the rod, or the rod itself may have some magnetic character. Stainless steel has only very low ferromagnetic properties, and titanium lacks any. Thus, suitable design considerations must be employed in this design.

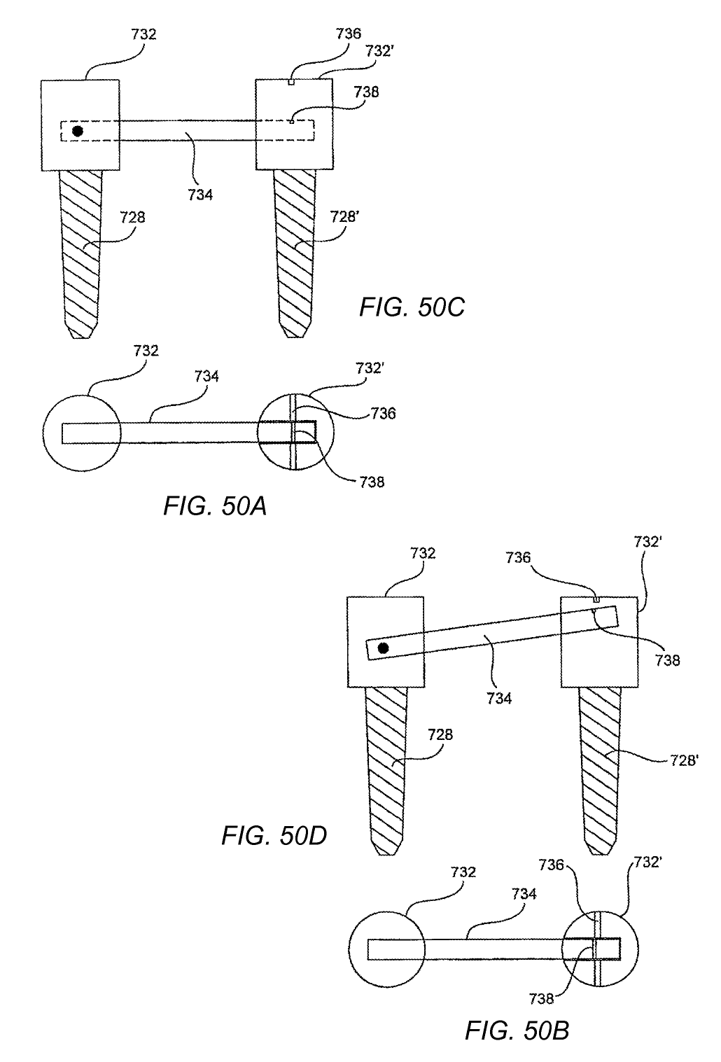

Other systems may employ radiopaque markings or markers to identify placement of the bone screws and the pivoting rod, and to confirm proper alignment of the distal end of the pivoting rod and the receiving assembly or cradle. In this case, of course, the other components would preferably be made of polymers to make the markers distinct. Referring to FIG. 50A-D, a system is shown with two bone screws 728 and 728′, each with a respective seat 732 and 732′. A pivoting rod 734 extends between the seats. A radiopaque marker 738 is shown on the pivoting rod 734 which, when in a deployed configuration, is disposed substantially in the center of seat 732′. Another radiopaque marker 736 is disposed in the center of the top face of seat 738. Each of the radiopaque markers extends linearly a predetermined distance. When viewing the system from the top, proper deployment of the pivoting rod is seen by co-linearity of the two radiopaque markers 736 and 738. If the radiopaque markers are parallel but not collinear, as seen in FIG. 50B, the pivoting rod may be determined to be not in a properly-deployed configuration. Of course, numerous other arrangements of radiopaque markers may be envisioned by those of ordinary skill in the art given this teaching.

The radiopaque markings or markers may include radiopaque fillers or dyes, tantalum beads or strips, etc. Alternative types of markers may also be employed, including those that are evident on MRI or ultrasound scans. These may include magnetic markers and ultrasonically reflective markers, respectively. Such markers may be employed to confirm proper placement, configuration, etc.

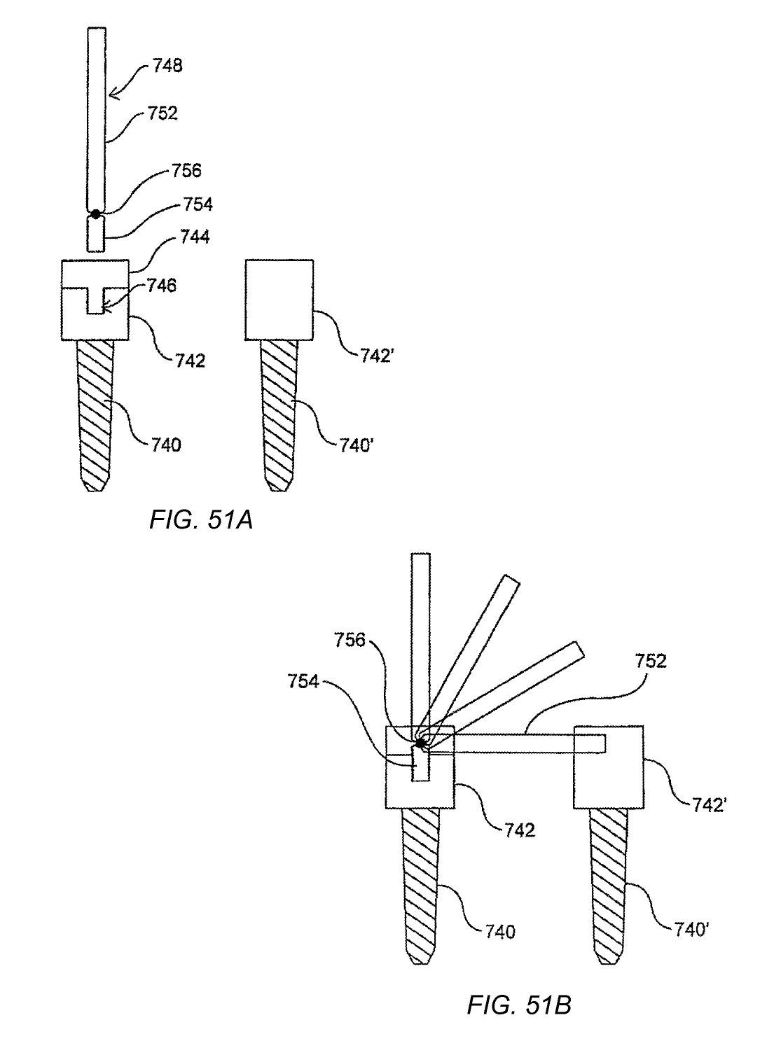

Several of the above systems describe configurations in which a hinge for a pivoting rod is provided in the seat attached to a bone screw. However, such a hinge may also form a part of the pivoting rod. Referring to FIG. 51A-B, two bone screws 740 and 740′ are shown with respective seats 742 and 742′. Seat 742 has a receiving assembly 744 including a threaded section 746. Of course, the threaded section could be integral with the seat 742 in an alternative embodiment.

Hinges in the embodiment of FIG. 51A-B may be designed with one degree of freedom or multiple degrees of freedom, and can include elements that limit travel such as various restricting devices. Such hinges can be adjustable by the physician, e.g., via a sliding rigid collar or partial collar, etc. In general, other hinge designs described, where the hinge forms part of a base or is formed in the attachment of the rod to the base or seat, may be carried over into this design.

A pivoting rod 748 is shown with an integral hinge 756. The pivoting rod has a pivoting section 752 and a threaded rod section 754. The threaded rod section 754 screws into the threaded section 746 to secure the rod into the seat. Following the securing, the pivoting rod may be pivoted and captured by a receiving assembly within seat 742′.

In an alternative embodiment, as noted above, the threaded rod section 754 could screw directly into the seat 742 or into a portion of the bone screw 740 (not shown). In this case, the threading of the threaded rod section 754 into the bone screw 740 could serve to further expand the bone screw, further anchoring the same into the pedicle.

The embodiment of FIG. 51A-B has the manufacturing advantage that the same screw design may be used for all pedicle screw and seat systems.

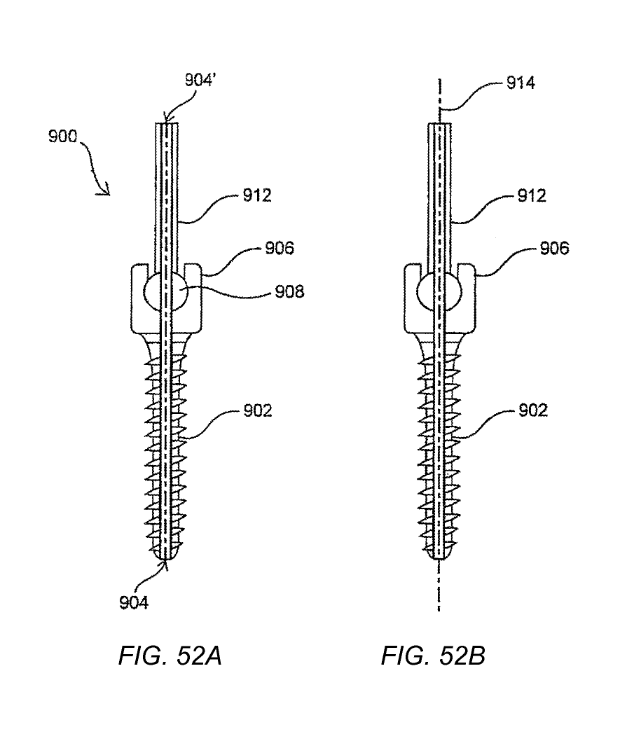

In all of the above systems, a guidewire lumen such as for a K-wire may be employed to assist in the installation of the system. Referring to FIGS. 52A-B, a system 900 is shown with a bone screw 902, a seat 906, a rod 912 coupled to a ball end 908 that is rotatably but fixedly installed in the seat 906, and a guidewire lumen having a distal end 904 and a proximal end 904′. The guidewire is shown as guidewire 914 in FIG. 52B.

In this system, the guidewire lumen extends from the proximal tip of the pivoting rod 912 to the distal tip of the screw 902. In other words, the assembled device is cannulated to allow the acceptance of a guidewire such as a K-wire. Generally, the lumen may have a uniform inner diameter through its length.

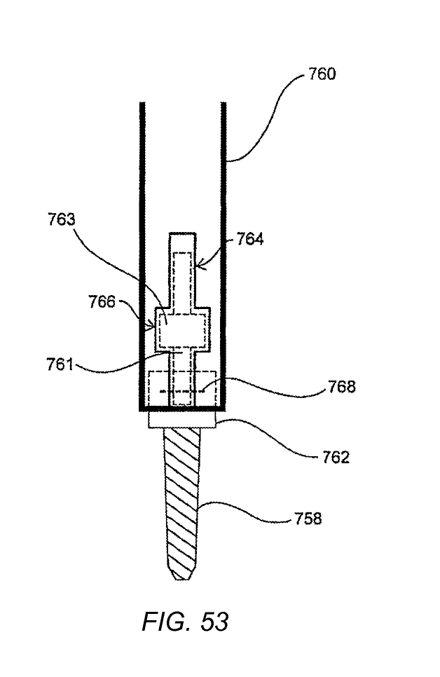

Systems as have been described may employ pivoting rods that have dynamic stabilization elements. Certain such “dynamic rods” may incorporate non-cylindrical or otherwise non-uniform shapes, such as a bulge, and as such may encounter difficulty when rotating out of an installation cannula for deployment. For example, referring to FIG. 53, a bone screw 758 is shown with a seat 762 having an axle 768 for rotation of a pivoting arm 761 having disposed within a dynamic stabilization element 763. While pivoting arm 761 and dynamic stabilization element 763 are shown with cylindrical cross-sections, the dynamic stabilization element 763 “bulges” with respect to pivoting arm 761, and thus would be difficult to slide down a cannula in a secure fashion. To address this situation, a cannula 760 is shown that has a void section 764 for a rod and a void section 766 that is substantially in the shape of the “bulge” of the dynamic stabilization element 763. Enough clearance should be provided between the dynamic stabilization element 763 and the void section 766 such that the pivoting rod 761, along with the dynamic stabilization element 763, may be rotated out of the cannula. In this case, the pivoting rod 761 would be rotated into or out of the plane of the figure for deployment.

The nature of dynamic stabilization element 763 may vary, and may include any functional such element. Of course, the system may be used with any pivoting rod that has a nonuniform part—it is not limited to dynamic rod systems.

It should be noted that the description above refers to specific examples of the invention, but that the scope of the invention is to be limited only by the scope of the claims appended hereto. Moreover, the sizes and materials shown for the components of the system may vary, but certain ranges of sizes and materials have been shown to be of particular use.

For example, the bone anchors, i.e., pedicle screws, shown may have exemplary lengths ranging from 25 to 80 mm, and may, e.g., be available within that range in 5 mm increments. The diameters of the same may be, e.g., 5.5 mm, 6.0 mm, 6.5 mm, etc. They may be made of metal, such as a titanium alloy, e.g., Ti-6Al-4V, ELI, etc. They may also be made of stainless steel, e.g., 316LSS or 22-13-5SS. The holes into which the same are inserted may be pre-tapped, or alternatively the pedicle screws may be self-tapping. If the bone anchor has a receiving slot, such as a hex head or other such head, then a screwdriver may be used to attach to the bone anchor directly. Once the pivoting rod is in place, a screwdriver may attach to the pivoting rod for further rotation. The pivoting rod itself may be used to further drive the screw.

The bone anchors may further have either fixed or polyaxial heads. Their threads may be standard, may be cutting threads, may incorporate flutes at their distal end, or may be any other type of thread.

The bone anchors need not be purely of a screw-type. Rather they may also be soft-tissue-type anchors, such as a cylindrical body with a Nitinol barb.

The pivoting rods or arms shown may have exemplary lengths ranging from 30 to 85 mm, and may, e.g., be available within that range in 5 mm increments. The diameters of the same may be, e.g., 5.5 mm, etc. They may be made of metal, such as CP Titanium Grade 2, stainless steel, etc.

The pivoting rods may be rigid or may also include a dynamic element, as is shown in FIGS. 9, 12, 13, 15, 17, and 18. In many of these embodiments, a spring or a spring-like mechanism forms a portion of the dynamic rod.

Moreover, the rod, whether dynamic or rigid, may be contoured prior to insertion. In other words, to more closely match the curvature of a spine, or for increased strength, i.e., to accommodate the geometry of the pedicle bone screws, or to accommodate the geometry of the spinal segment in which it is installed, a curve or other contour may be designed into the rod prior to insertion. Alternatively, a physician may bend the rod or put another such contour into the rod, either manually or with the aid of a device, prior to insertion.

While the multi-level systems have been shown with rods that are substantially the same size and shape, there is no inherent need for such similarity. The rods can vary in length, diameter, or both. Moreover, the rods can be non-dynamic or can employ dynamic elements.

Further, systems according to the disclosed embodiments may be disposed not only on multiple levels of the vertebrae but also on different sides of the spinous process. In other words, two systems may be disposed in a single segment, one on each pedicle. Moreover, the use of the disclosed pedicle-screw-based systems may be employed in combination with various spacer systems. The guidewire lumen configuration of FIG. 52 can be used with other spinal systems, such as facet devices, dynamic linking devices, etc.

Cannulae such as those described in connection with FIG. 53, or indeed any cannulae, should generally be such that the last, largest, cannula, is as small as possible but large enough to accommodate passage of the large OD device within. A large dilator such as this may have a outer diameter of, e.g., 13.0 mm. The first cannula, that initially slides down the K-wire or other guide, may have an inner diameter of, e.g., 1.6 mm.

The first or a later cannula may be configured to mate with the hinged assembly, i.e., the pivoting rod assembly, in order that the cannula can be used to direct the slot (for the pivoting rod) into the proper orientation. To this end as well, the cannulae may have markings on their proximal end to indicate the orientation of the slot. The second or later-used cannulae need not have a slot to allow movement of the pivoting rod—rather they may be withdrawn a short distance, e.g. a distance slightly greater than the length of the pivoting rod, to allow the rod to pivot through the tissue and into a deployed configuration and into a receiving assembly.

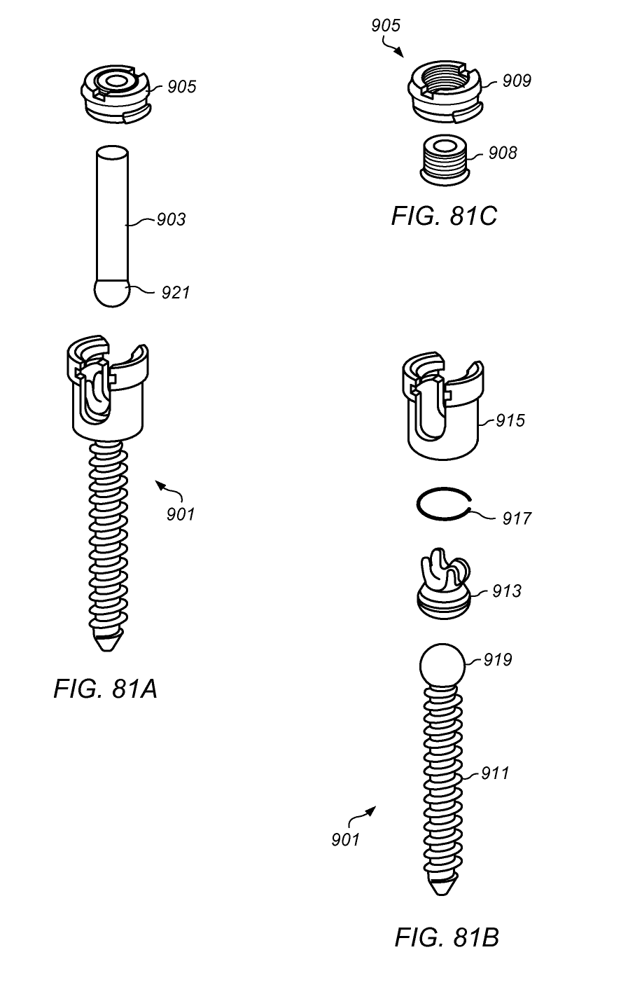

FIG. 81A shows an exploded view of one embodiment of the bone stabilization device, which is similar to the embodiment depicted in FIG. 24. The bone stabilization device includes a screw assembly 901, pivoting rod 903 and cap assembly 905. As shown in FIG. 81B, the screw assembly includes a screw 911 with screw head 919 which matingly engages with a pivoting element or coupler 913. The coupler 913 engages with the seat 915 using retaining ring 917. The seat 915 has two partially-spherical voids formed within to accommodate a hinge pin 921 located at the base of the rod 903. After the rod is pivoted into position for use in a patient, the rod is held in that position by a cap assembly 905 shown in FIG. 81C, which is defined by cap 907 and setscrew 909. The cap assembly 905 may be fitted into seat 915 using grooves or the like. Further details of the embodiment shown in FIG. 81 may be seen by reference to the previously described embodiments, in which similar elements have similar descriptions and functions. Prior to installing the bone stabilization device into a patient, the cap assembly 905 and the screw assembly 901 are pre-assembled for each of the pedicles in which they are to be installed.

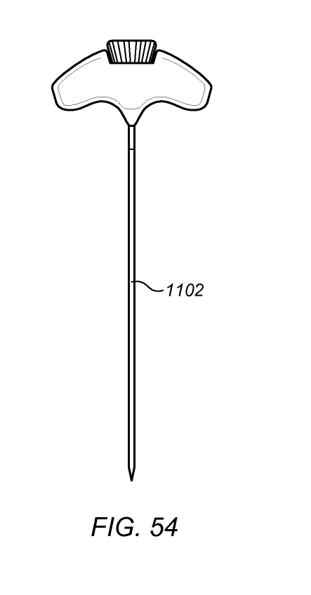

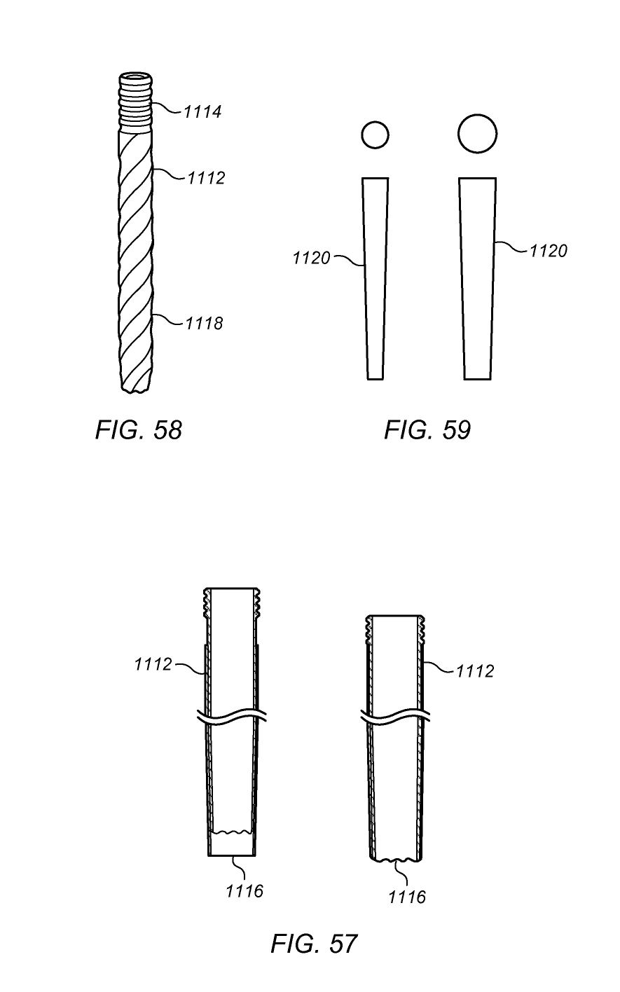

FIGS. 54-82 illustrate a system of tools that may be used to place the bone stabilization device of FIG. 81 in a minimally invasive percutaneous procedure. A procedure using these tools will then be presented to further facilitate an understanding of the systems, tool, and procedures of the present invention.

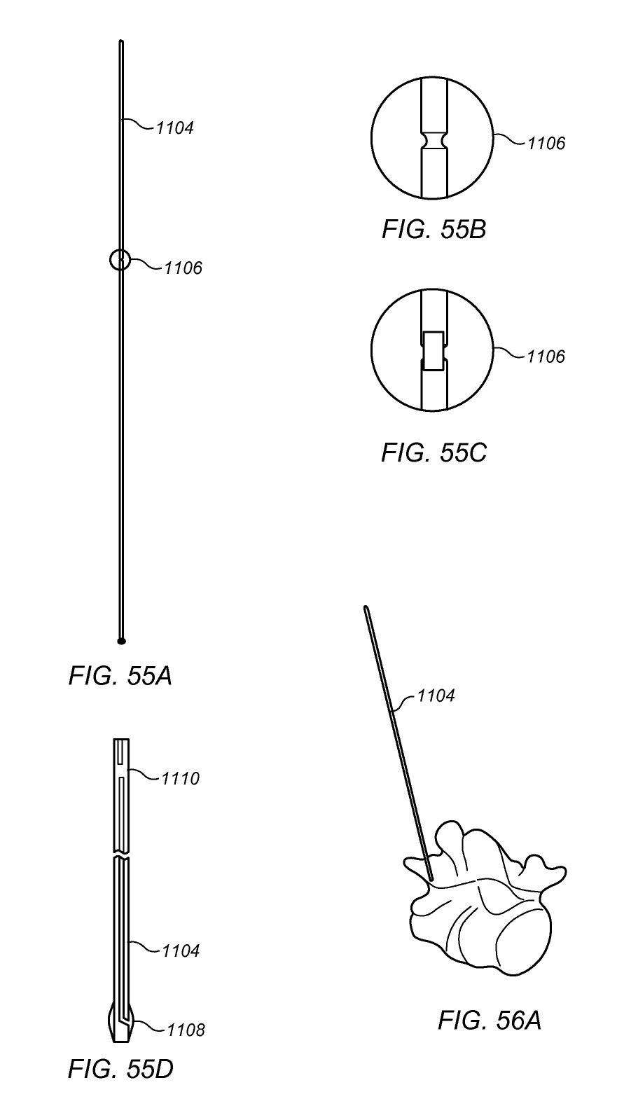

The procedure begins with a guidewire placement procedure depicted in FIGS. 54-55. FIG. 54 shows a target needle 1102 that is used to penetrate through the skin up to and through the pedicle. The target needle 1102 has an inner needle portion that is removable while leaving an outer guide in place. A guidewire 1104 is inserted through the outer guide of the target needle 1102. In an alternative embodiment, the inner needle portion of the target needle 1102 may be cannulated, allowing the guidewire to be inserted through it without removal. In this alternative embodiment, the needle may be partially withdrawn, e.g. to retract the sharp tip, prior to guidewire advancement The guidewire 1104, shown in FIG. 55A, may be similar to a conventional guidewire that is used for over-the-wire insertion and exchange of various cannulated devices. The guidewire 1104 may include a depth marker 1106 (e.g., a groove or band such as depicted in FIGS. 55B-C, respectively) to indicate how far it has penetrated. Alternatively or additionally, markers may be included in guidewire 1104 or target needle 1102, such as visible markers, radiopaque markers, ultrasonically reflective markers, magnetic markers and other markers. In one alternative embodiment, depicted in FIG. 55D, the guidewire 1104 may include an expandable tip 1108 such as a balloon or cage. The expandable tip 1108 serves as an anchor in the vertebra, thereby preventing the guidewire 1104 from advancing through the anterior side of the vertebra and/or pulling out of the vertebra. If a balloon is employed, the guidewire 1104 may employ a thru-lumen with a valve 1110 on its proximal end to releasably maintain the pressure in the balloon. The guidewire 1104 may also have a flexible tip to prevent advancement through the anterior side of the vertebra and a retractable sharp tip for purposes of advancement. In an alternative embodiment, guidewire 1104 includes a retractable, sharpened tip, which can be selectively advanced to assist in penetration through bone. After the guidewire 1104 has been properly placed, the target needle 1102 can be removed from the patient.

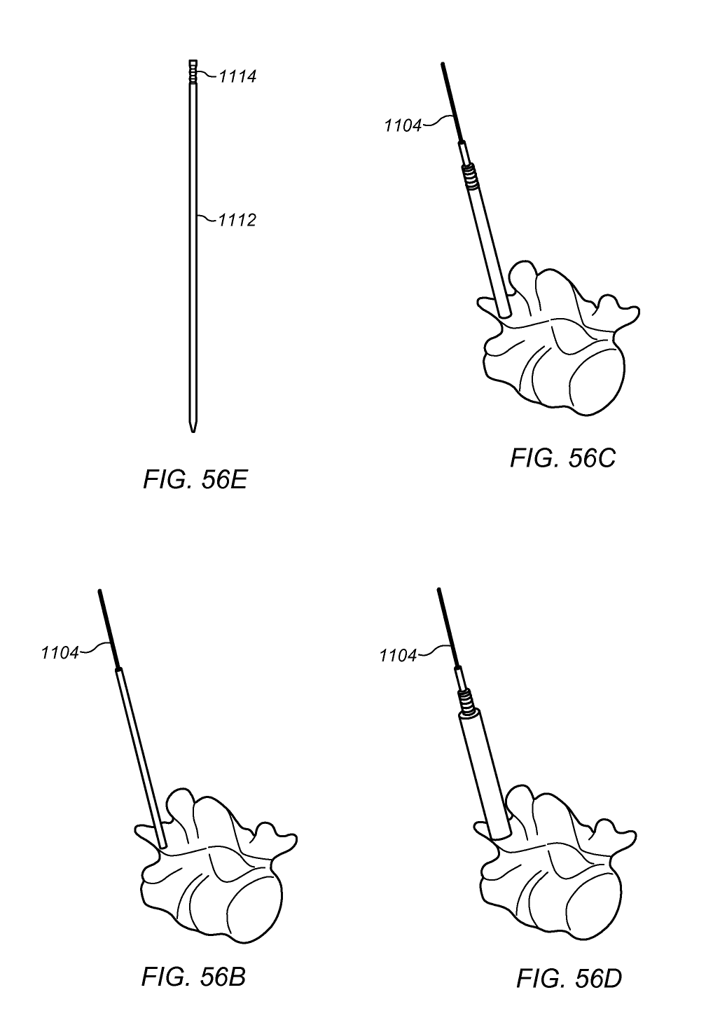

A series of cannulated dilators are employed to sequentially dilate and expand the tissue between the entry site established by the target needle 1102 and the pedicle. An example of such a dilator is shown in FIGS. 56A-E. The dilator 1112 may be provided with a knurled end 1114 for the operator to grip. The dilators fit one over the other in increasing order of diameter. For instance, if three dilators are employed, the dilator with the smallest diameter advances over the guidewire 1104, the dilator with the intermediate diameter advances over the smallest diameter dilator and the dilator with the largest diameter advances over the intermediate diameter dilator. Each dilator has an ID/OD selected so that it mates with both the corresponding smaller and larger dilators. As shown in FIGS. 56A-E and 57, some or all of the dilators 1112, particularly the largest dilator, may have advancable grippers such as retractable teeth 1116 on their distal ends to provide a gripping force when pushed against bone or other tissue. In an alternative or additional embodiment, the teeth 1116 can be used to cut through tissue as the dilator 1112 is advanced. The grippers are preferably configured to be deployed only when needed. In some embodiments, depicted in FIG. 58, the dilators 1112 may have helical grooves 1118 on their outer diameters to assist in advancement through tissue. The dilators 1112 may also be provided with depth, tip or other markings, which may include, for example, visible, radiopaque, ultrasonically reflective, or magnetic markers. Alternatively, the markers may be formed from grooves or bands formed in the dilator 1112. In other embodiments, an expandable or tapered dilator is provided. As shown in FIG. 59, the expandable dilator 1120 increases in diameter from its distal end to its proximal end. The expandable dilator can be formed from a rolled sheet such as a flexible metal (e.g., nitinol, spring-steel, etc), which has preferably been rolled into a tube that may or may not be tapered. During or after insertion, the tube is “unrolled”, manually or with an end-gripping, torque tool (not shown) that causes the outside end of the sheet to rotate relative to the inside end of the sheet), thus increasing the diameter of the tube. This embodiment allows insertion of a small diameter dilator, OD increase of the dilator and further dilation of tissue while the dilator is in place, which transforms to a larger dilator without insertion of a 2^{nd} dilator. The expandable dilator 1120 may include any of the aforementioned features such as advancable grippers, retractable teeth and the like.

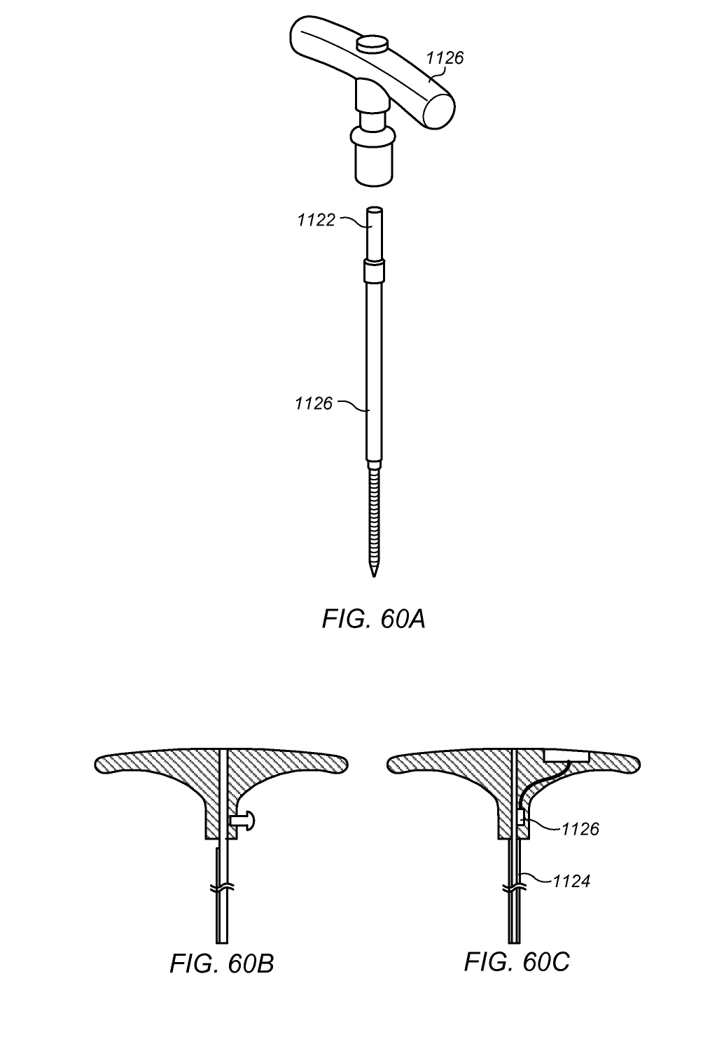

FIG. 60A shows a tap device 1122 that is used to tap a hole in the bone in which the screw 901 will be implanted. The tap device is placed over-the-wire and through the large diameter dilator and positioned up to the pedicle surface. The tap device 1122 is a two part assembly comprising a handle 1124 and a tap drive 1126. A variety of different handle types may be employed such as a T-handle, axial and ratchet, for example. Alternatively, the handle 1124 and tap drive 1126 may be formed as an integral unit. The tap 1126, which may be available in multiple sizes, is cannulated for over-the-wire use. Alternatively, the tap 1126 may be a solid structure so that it can be used with smaller size screw e.g., 4.0-5.0 mm). Rotation of the tap device 1122 creates a threaded hole for insertion of the pedicle screw assembly 901. The tap 1126 contains a trocar style point. The trocar creates a slightly undersized hole in the bone to help ease the cutting flutes into the bone to start the tapping process. This way bone is removed incrementally in a way that reduces stress so the bone or pedicle is not fractured. This provides a snug and secure fit between the bone and the screw. The thread of the tap may be slightly undersized so that the self tapping flute of the screw cuts the final path into the bone for a snug and secure fit. Alternatively, instead of the tap 1126, self-tapping pedicle screws may be employed. The tap device 1126 may include an operator releasable clamp to prevent undesired movement of the guidewire and avoid the need for a separate guidewire clamp. In some embodiments the tap handle 1124 and/or tap device may include a measurement assembly such as an optical motion sensor and a visual display to indicate the relative movement of the device relative to the guidewire. Among other things, the measurement assembly can allow measurement of the drilled hole to determine an appropriate pedicle screw length. In FIGS. 60B-C the handle 1124 is shown with an integrated optical motion sensor 1126 and a visual display 1128. The tap 1126 may also be provided with markings such as to indicate the depth to which the tap has been inserted, which can be correlated to the appropriate pedicle screw length. The markers may include, for example, visible, radiopaque, ultrasonically reflective, or magnetic markers.

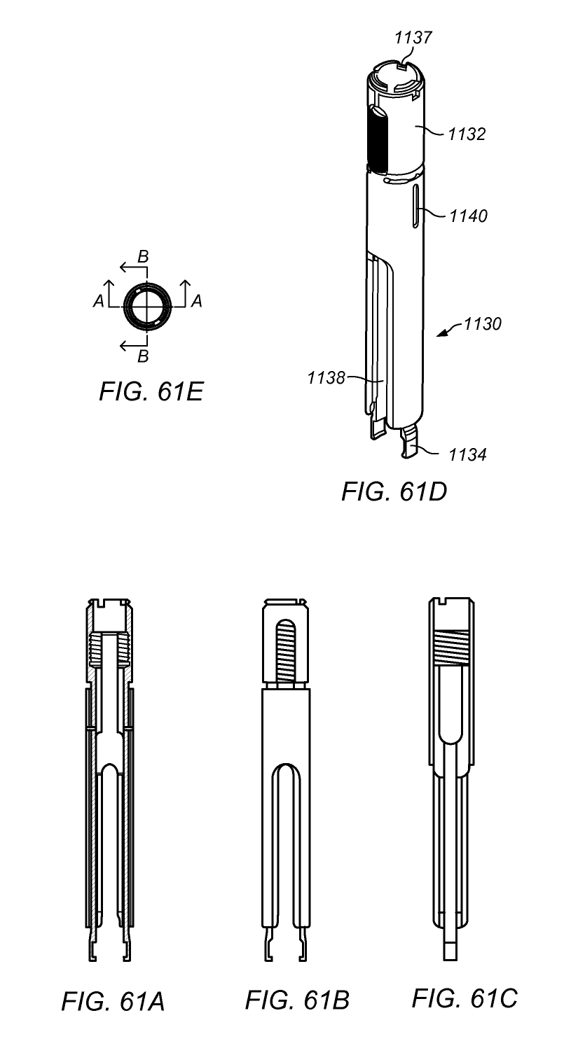

FIGS. 61A-E show a screw tower assembly (STA) tool 1130 that is used to insert the pedicle screw assembly 901. The STA effectively becomes a working channel through which the remaining components (e.g., rod 903, cap) of the bone stabilization device will be inserted. The STA 1130 has a generally tubular configuration with an externally threaded bushing 1132 in its proximal end and extendable/retractable tangs 1134 on its distal end to which the screw assembly 901 is secured. The proximal end of the tower and the bushing 1132 has two or more notches 1137 (four are shown in FIGS. 61A-E) that allow for the keyed insertion of various other devices such as a locking tool and a screwdriver, both of which will be described below. Alternative attachment mechanisms may be included on the proximal end of STA 1130, such as an internally threaded bushing, frictional engagement collar, bayonet lock, magnetic attachment assemblies, and other mechanisms used to attach a hand-held device to the tubular structure of the STA 1130. The bushing 1132 and tangs are arranged in a mechanically cooperative manner so that rotation of the collar 1132 extends and retracts the tangs 1134, which secure the screw to STA 1130. The distal end of the STA 1130 may also be sharpened, include grippers, or the like. A rod channel 1138 is formed in the tubular body of the STA 1130 and extends to the distal end of the STA 1130. The rod channel 1138 provides an exit pathway for the rod 903 so it can be pivoted about its base 921 from a location within the STA 1130 and into the adjacent screw assembly 901. The rod channel 1138 can also serve as an alignment marker and is preferably oriented in a cephalad-caudal alignment through the procedure. A vertical line 1140 or other marker may be provided on the proximal end of the STA 1130 that allows the rod channel 1138 to be properly aligned with the primary and secondary alignment guides 1154 and 1160, which are described below.

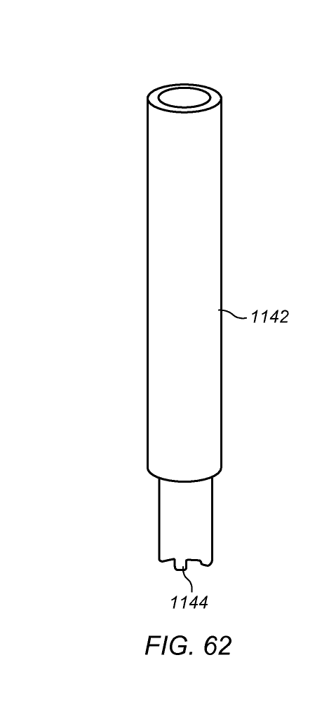

FIG. 62 shows a locking tool 1142 having a tubular body that includes engaging lugs 1144 on its distal end. The engaging lugs 1144 mate with the notches in the STA 1130 (see FIG. 61) so that the locking tool 1142 is operatively attached to the STA 1130. The locking tool 1142 serves as a rotational device that allows relatively large torsional forces to be exerted on various tubular devices to which it connects. The locking tool 1142 can also be operatively attached to the primary and secondary access guides and the rod introducer, all of which will be described below. In some case the locking tool 1142 may be integrally formed with the STA 1130 or any of the other devices to which it connects. Instead of the engaging tangs 1144 the locking tool 1142 may employ other attachment means such as threads, a male-female slip fit engagement arrangement, or the like so that it can be operatively attached to the various other devices. The locking tool 1142 may also be provided with markings to indicate depth, orientation, alignment or other information. The markers may include, for example, visible, radiopaque, ultrasonically reflective, or magnetic markers.

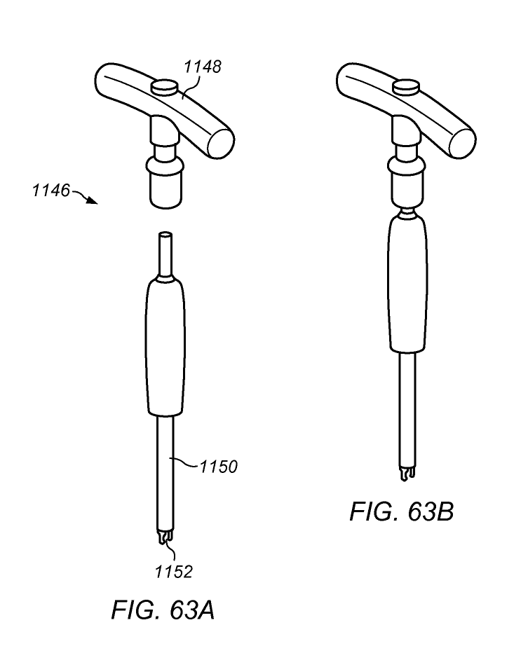

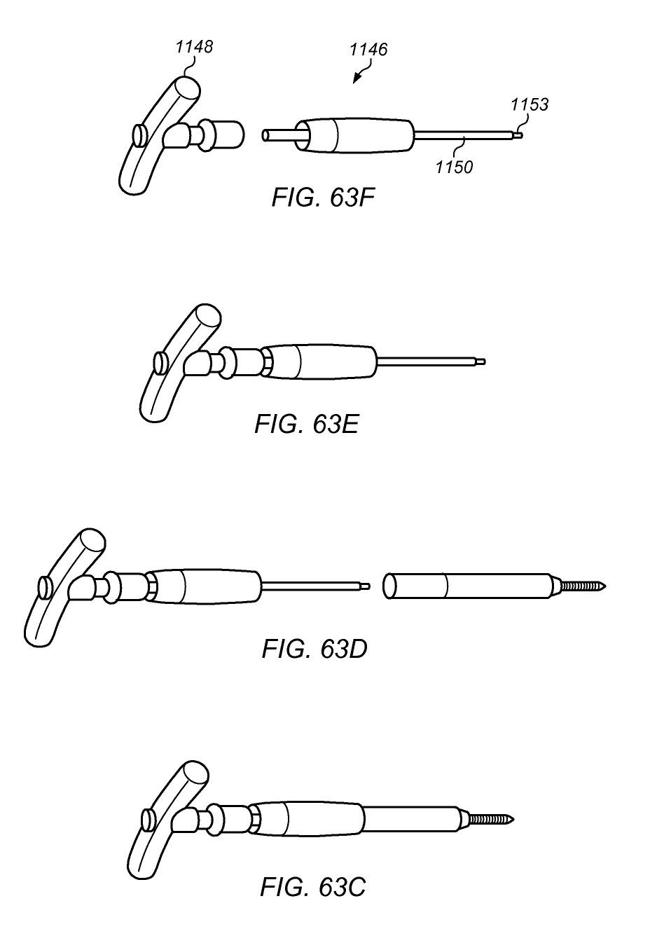

FIGS. 63A-F show a polyaxial screwdriver 1146 that includes a handle 1148 and a tubular body 1150 to which the handle 1148 attaches. The engagement mechanism employed by the screwdriver 1146 may comprise tangs (FIG. 63A) or a hex driver 1153 (FIG. 63B). The tubular body 1150 can act as an operator grip location, which allows the operator to hold screwdriver 1146 while the handle 1148 and tubular body 1150 are being turned. Gripping along the tubular body 1150 allows the operator to independently orient the channel in the STA while turning the handle 1148 and shaft to insert the screw. The handle 1148 may include an operator engageable/releasable clamp to prevent movement of the guidewire, thereby avoiding the need for a separate guidewire clamp. The polyaxial screwdriver 1146 is inserted through the proximal end of the STA 1130 and engages with the screw assembly 901 that is held in place at the distal end of the STA 1130 by the tangs 1144 (see FIG. 62). The screwdriver 1146 is inserted over-the-wire with the STA 1130 and the screw assembly 901. Rotation of the screwdriver 1146 inserts the screw assembly 901 into the pedicle. The tubular body 1150 has a proximal end that allows for quick connect with the handle 1148 and a mid-portion that can serve as an operator grip point that also is used to orient the channel of the STA, such as to pivot the rod from screw to screw. The tubular body 1150 is cannulated. The distal end of the screwdriver 1146 has an inner diameter sized to slidingly receive the proximal end of the STA 1130. A tang may be provided so that the distal end of the screwdriver 1146 mates with the notches 1137 in the proximal end of the tubular body 1132 of the STA 1130. A locking mechanism may be provided to lock the screwdriver 1146 to the STA 1130. The locking mechanism can hold the STA 1130 to prevent it from disengaging as the screwdriver 1146 is passed over the guidewire. The distal end of the screwdriver 1146 has a generally smaller diameter than its proximal end. The engagement mechanism (e.g., tangs 1152 or hex driver 1153) located on the distal end of the screwdriver 1146 pass though the coupler 913 of the screw assembly 901 (see FIG. 81A). The engagement mechanism engages with the spherical head 919 of the screw 911. Both the handle 1148 and the tubular body 1150 may include linear markers so that after the final rotation of the screw assembly there is proper alignment with the rod channel 1138 of the STA 1130. That is, the linear markers can be used to confirm that the screw heads are appropriately aligned with the spine such that when the pivoting rod is inserted into the first screw assembly it 903 will pivot towards the second screw assembly. The screwdriver 1146 may also be provided with depth, tip and other markings. The markers may include, for example, visible, radiopaque, ultrasonically reflective, or magnetic markers.

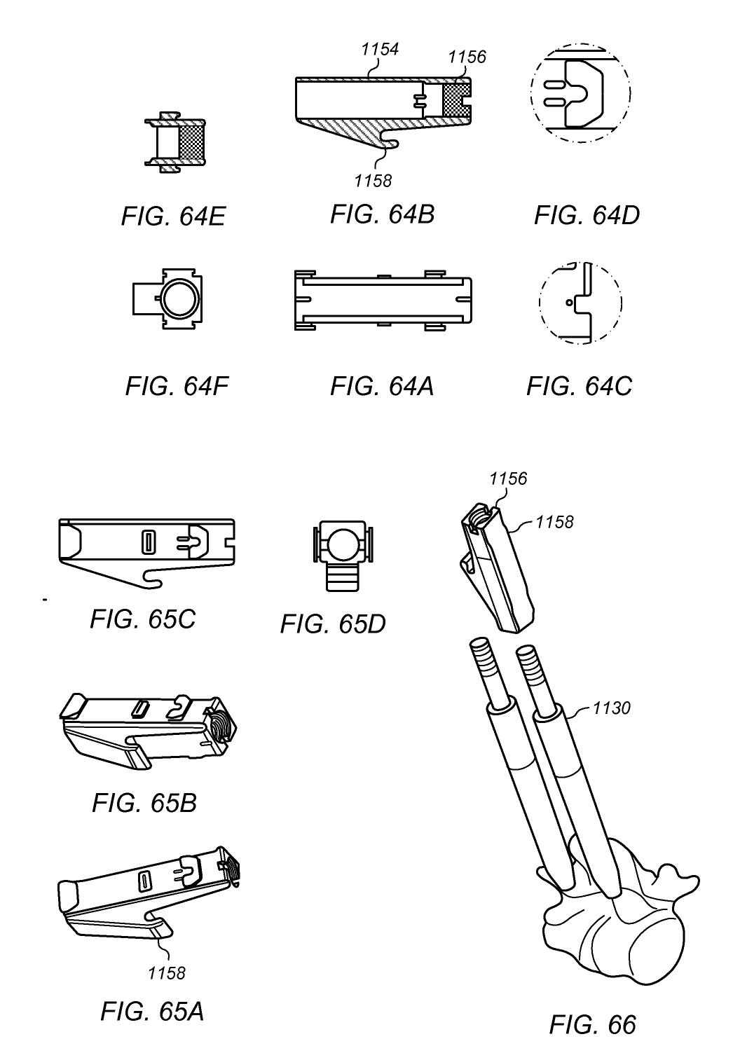

FIGS. 64A-F and 65A-D show perspective views of a primary alignment guide 1154 that is employed to align the seat 915 of the screw assembly 901 so that the rod 903 can be received by the coupler 913 using a rod introducer assembly. It is also used to receive the rod measuring instruments (described below), tissue splitter (described below), rod introducer (described below) to introduce and insert the rod 903, rod pusher (described below) to pivot the rod once inserted, cap inserter (described below) to insert and provisionally tighten the cap assembly 905, to mount the distraction/compression tool (described below), The distal end of the primary alignment guide 1154 fits over the proximal end of the STA 1130, as shown in FIG. 66, and is secured thereto with the locking tool 1142 The primary alignment guide 1154 may also have an internal bushing at its proximal end, with notches that are used to secure it to the proximal end of the STA 1130. Markers may be provided to ensure that the primary alignment guide 1154 and the STA 1130 are properly aligned. The proximal end of the primary alignment guide 1154 has internal threads 1156 to receive the rod length measuring tool, the torque indicating driver, the tissue splitter, rod pusher and the cap inserter, which are described below. A mechanical alignment mechanism (e.g. notches, lugs, tangs, etc.) may be provided to ensure that the aforementioned tools are properly aligned. A hook 1158 extends outward from a mid-portion of the primary alignment guide 1154. The hook 1158 mates with a cross pin in the secondary alignment guide 1160, described below, to form a hinge therewith. The hinge allows the alignment guides to be coupled so the seats of the polyaxial screws are aligned to accept the rod during insertion. The hinge also allows for distraction or compression forces to be applied to the instruments to adjust the distance between the vertebra segments such as to restore proper disc height and relieve impingement of soft tissue structures.

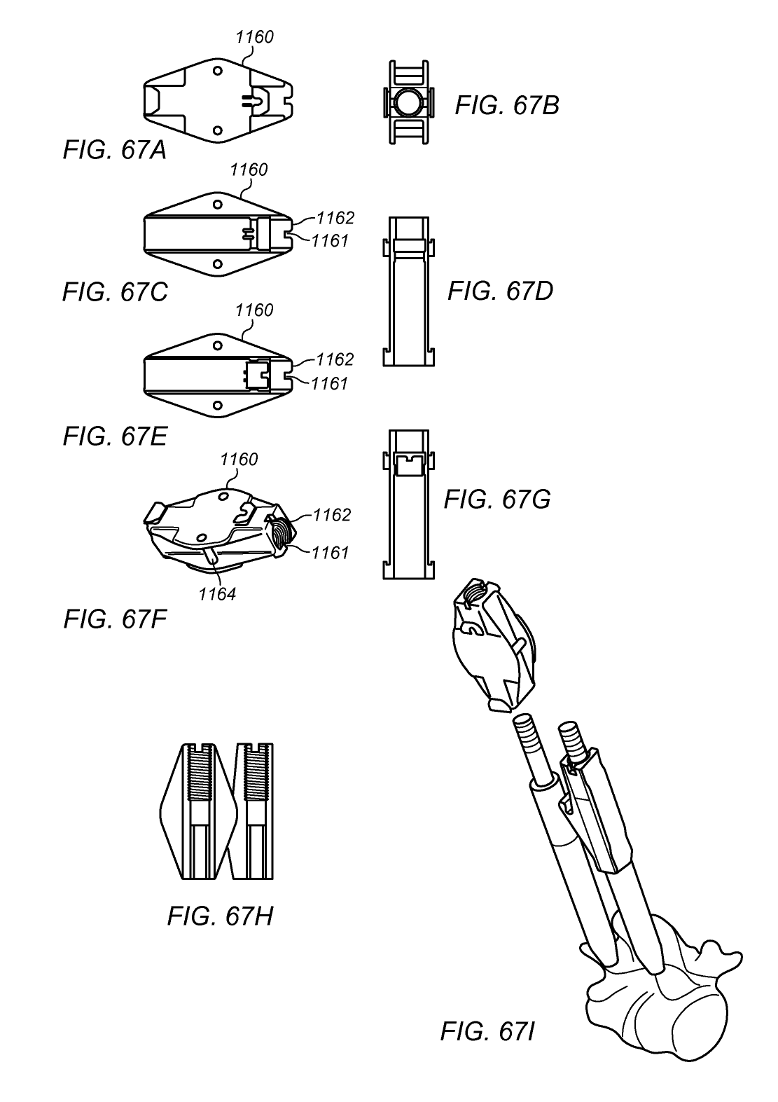

FIGS. 67A-I show various views of a secondary alignment guide 1160. The secondary alignment guide 1160 fits over the proximal end of a second STA 1130 that is positioned with a screw in the pedicle of a vertebra either above or below the vertebra in which the first STA 1130 is positioned. The locking tool 1142 is used to secure the second alignment guide 1160 to the STA 1130. An internal bushing in the secondary alignment guide 1160 has notches 1161 that mate with the locking tool 1142. The lugs 1144 of the locking tool 1142 engage with the notches of the bushing. Rotation of the locking tool causes the bushing to advance and lock the secondary alignment guide 1160 to the STA 1130. The Secondary Alignment Guide 1160 has an elongated hexagonal shape with a cannula extending through its body. The distal end of the through cannula is designed to accept and attach to the proximal end of the screw tower assembly 1130. As described in more detail below, at the hex points located at the mid-point of the body a cross pin 1164 is provided that engages with the hook 1158 of the primary alignment guide so that the seats 915 of the screw assemblies are pivotably aligned with one another to accept the rod The proximal end of the secondary alignment guide 1160 includes internal threads that mate with the tissue splitter, the rod introducer, the rod pusher and the cap inserter. A mechanical key is also provided so that the tissue splitter, the rod introducer, the rod pusher and the cap inserter are properly aligned when mated with the secondary alignment guide 1160. The secondary alignment guide 1160 may also be provided with depth, tip and other markings. The markers may include, for example, visible, radiopaque, ultrasonically reflective, or magnetic markers. Horizontal or vertical linear markers may also be provided to align or orient with other tools such as the rod channel 1138 of the STA 1130.

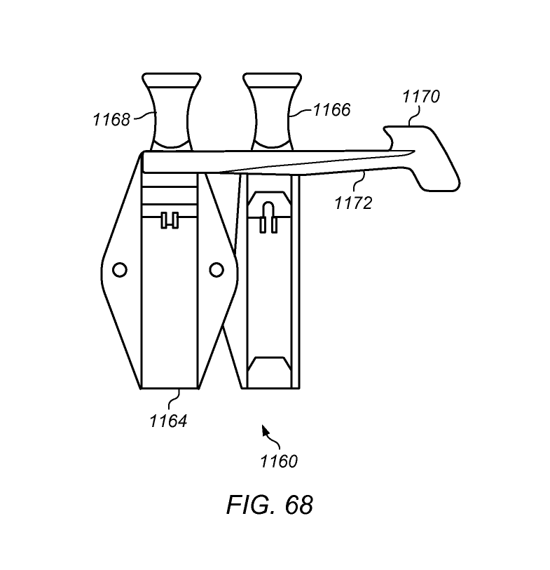

As previously mentioned, the secondary alignment guide 1160 is pivotably attached to the primary alignment guide 1154. In the particular embodiment of the secondary alignment guide shown in FIGS. 67A-I, a cross pin 1164 is provided at the mid-point of the secondary alignment guide body. The cross pin 1164 extends through the body from one end face to the other in a direction perpendicular to the longitudinal axis of the body. The cross pin 1164 fits over the hook 1150 of the primary alignment guide 1154 to define a pivot or hinge that allows rotational movement of the secondary alignment guide 1160 relative to the primary alignment guide 1154 (see FIG. 68).

In some embodiments of the invention the proximal ends of primary and second alignment guides 1154 and 1160 may include alternative attachment mechanisms such as, without limitation, external threads or externally threaded collars, internally threaded collars, frictional engagement collars, bayonet locks, magnetic attachment assemblies, keyed (rotationally oriented) attachment mechanisms, and other mechanisms used to attach a hand-held device to the primary and second alignment guides 1154 and 1160.

In some embodiments of the invention the primary and second alignment guides 1154 and 1160 may be formed as a single unit.

FIG. 68 shows a rod length measuring tool that is used to determine the appropriate rod length that should be used. The rod length measuring tool measures the pivot angle of the pivot or hinge formed between the primary and secondary alignment guides 1154 and 1160. Based on the angle that is measured, the appropriate rod length that is needed can be determined. The rod length measuring tool includes a rod gauge indicator 1168 that is attached to the secondary alignment guide 1160 and a rod gauge measurement device 1166 that attaches to the primary alignment guide 1154. The rod gauge measurement device 1166 and rod gauge indicator 1168 slidingly engage with the primary alignment guide 1154 and the secondary alignment guide 1160, respectively, using the mechanical keys that are provided. The rod gauge indicator 1168 includes a gauge 1170 on which the pivot angle is indicated by a pointer 1172. In some cases the rod gauge indicator 1168 may include a mechanical or electronic rotary encoder that converts the angle into a value that represents the rod length that is required. If the rotary encoder is electronic, the value for the length of the rod may be converted into an electronic signal. An electronic module may be provided to receive the electronic signal from the rotary encoder and convert it into information representing the appropriate rod length. The rod gauge measurement device 1166 and rod gauge indicator 1168 may also be provided with depth, tip and other markings. The markers may include, for example, visible, radiopaque, ultrasonically reflective, or magnetic markers.

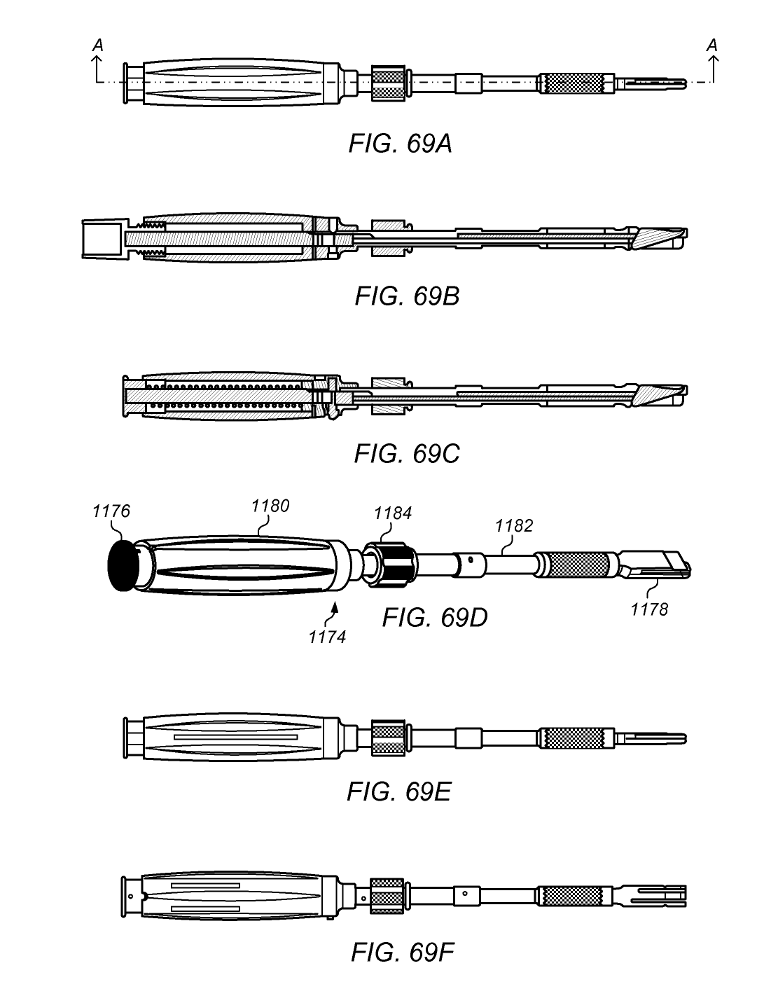

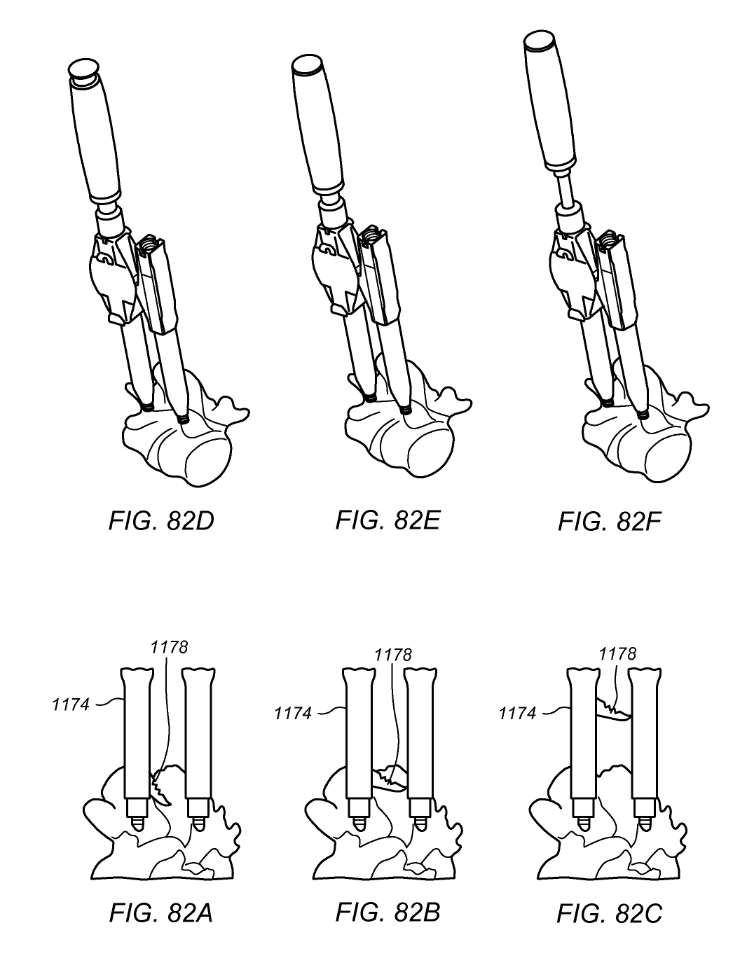

FIGS. 69A-F show a tissue splitter 1174 that is used to dissect the tissue between the seats of the screws so that a subcutaneous path is created for the rod to rotate into position between the screws once one end of the rod is secured in one end of the screw seats. The tissue splitter 1174 is passed through the primary alignment guide 1154 and/or the secondary alignment guide 1160 and is secured by threads. A button 1176 or other actuator located on the proximate end of the device is provided to extend a blade 1178 that is located on the distal end of the device. As seen in FIGS. 69A-F, the handle 1180 is attached to an elongate shaft 1182. A rotatable collar 1184 located on the proximate end of the shaft 1182 has external threads that engage with the primary or secondary alignment guide 1154 and 1160. The distal tip 1178 is shaped so that it can pass through the screw tower assembly 1130 in a single orientation. That is, the distal tip is a lug. The tip of the tissue splitter 1174 fits into the polyaxial seat 915 of the screw assembly 901 to determine the correct orientation of the instrument for actuation. Alternatively, the shaft 1182 may include a projection or lug that serves to orient the instrument by mating with the primary or second alignment guides 1154 and 1160 for proper alignment. The shaft 1182 slides through the rotatable collar 1184 to move the blade 1178 so that it cuts the tissue when pulled upward. As shown in FIGS. 82A-F, when the blade is extended it is oriented at 45 degrees with respect to the axis of the shaft 1182 (FIG. 82B). When the handle 1180 of the tissue splitter 1174 is pulled the blade 1178 is pulled upward along the axis of the shaft 1184 while maintaining the 45 degree angle to create friction along the edge of the blade 1178 to split the tissue (FIG. 82C) to create the path for the rod 903. An indicator may be provided to depict the position of the blade 1178. The blade 1178 itself may be provided with markers such as holes or the like that serve as a reference for determining the distance between the screw assemblies 901. Since the blade can be seen on fluoroscopy during the procedure, the blade outline can acts as a marker for the operator. The shaft 1182 may be provided with depth, tip and other markings. The markers may include, for example, visible, radiopaque, ultrasonically reflective, or magnetic markers. In some cases the tissue splitter 1174 may be energy assisted using, for example, RF energy, to facilitate cutting. In some alternative embodiments the blade may cut through tissue by pushing on the handle 1180 rather than pulling. This can be accomplished, for instance, by orienting the sharp side of the blade 1178 away from the operator instead of towards the operator as in FIGS. 82A-C. In other embodiments the shaft 1182 may be flexible with a trocar point that pushes down. When the shaft bends and extends toward the other screw assembly tissue is cut with the trocar edges during the advancement process.

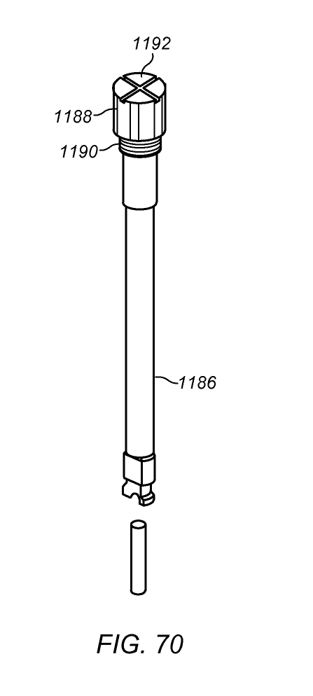

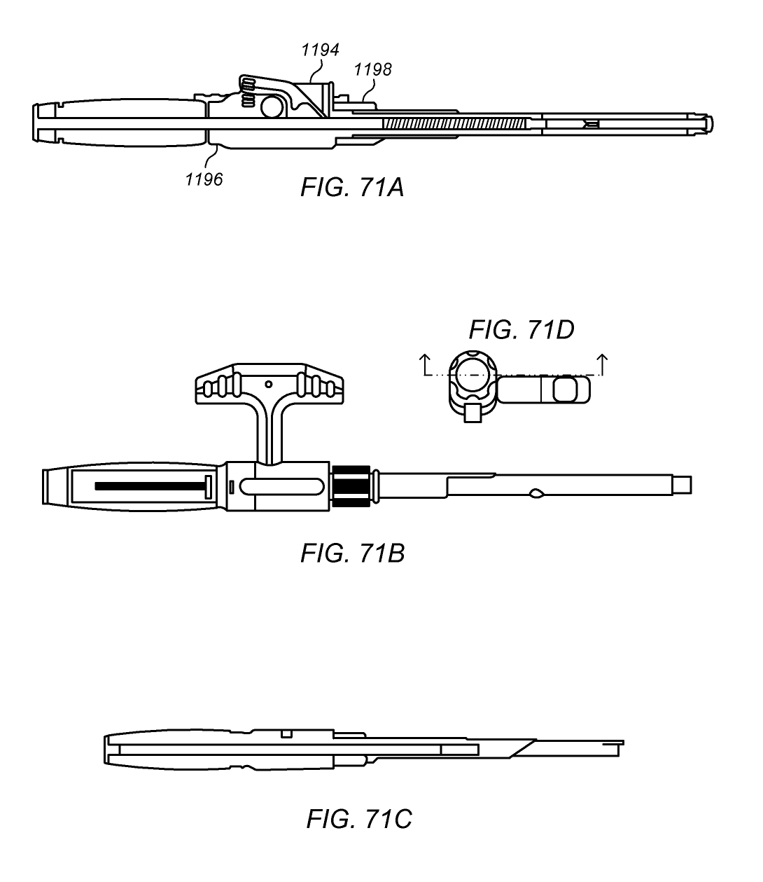

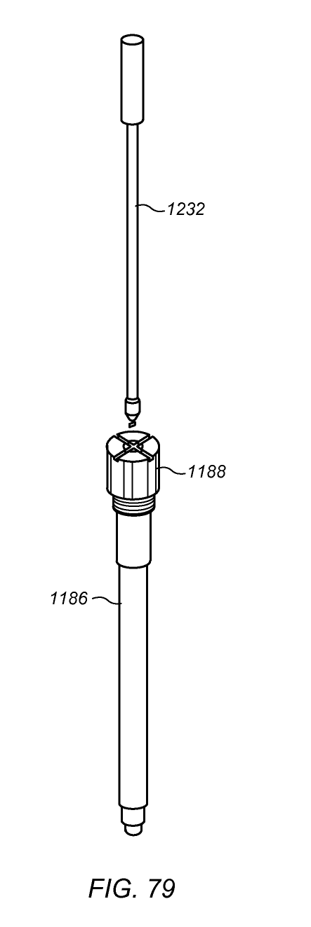

FIG. 70 shows a rod introducer assembly 1186 that is used to implant the rod 903 after the screw assemblies have been inserted. The rod 903 is slidingly received by the distal end of the assembly 1186 and held in place by a frictional fit, possibly with the use of an o-ring that surrounds and compresses the rod 903. Alternatively, the distal end of the assembly 1186 may include threads that engage with the rod to hold it in place. In other cases the distal end of the assembly may be magnetized to hold the rod in place. In yet another alternative, shown in FIG. 79, a separate rod holder 1232 may be inserted through the cannula of the rod introducer assembly 1186 to hold rod 903 in place. The rod introducer assembly 1186 is inserted through the primary or secondary alignment guides 1154 and 1160 and the screw tower assembly 1130 and into the coupler 913 of the screw assembly 901. The proximal end of the introducer assembly 1186 includes a rotating collar 1188 having external threads received by the threads of the primary and secondary alignment guides 1154 and 1160. The rotating collar 1188 includes notches 1192 that mate with the locking tool or other driving and/or pushing tool(s). By rotating the collar 1188 the rod is pushed into the coupler 913. The rod 903 is advanced until it engages with the seat/coupler 915/913 of the screw assembly 911. Once the rod 903 is secured the rod introducer assembly 1186 is removed. (The assembly 1186 is configured so that it can only be inserted through the STA 1130 in a single orientation so that the lugs on the base 921 of the rod 903 properly engages with the coupler and secures the rod to the screw assembly. The rod introducer assembly 1186 may also be provided with depth, tip and other markings. The markers may include, for example, visible, radiopaque, ultrasonically reflective, or magnetic markers. Other markers or the like may be provided on the shaft of the rod introducer assembly 1186 to align it with the primary or secondary alignment guides 1154 and 1160 before it is pushed into the coupler 913. FIGS. 71A-D show the rod pusher 1194, which is used to pivot rod 903 into position so that the rod is engaged with both screw assemblies 901. The rod pusher 1194 fits into the cannula of either the primary or secondary alignment guide 1160. A handle 1196 is rotated to pivot the rod toward the second screw assembly. The shaft of the rod pusher 1194 is keyed so that it only fits into the cannula with the proper orientation. A threaded collar 1198 secures the rod pusher 1194 to the secondary alignment guide 1160 during the operation. Rotation of the handle 1196 turns a pinion to engage and actuate a rack that pushes on a shaft or piston. As the shaft advances it pivots a member on a linkage at the distal tip to drive and pivot the rod into the adjacent screw assembly. This pivoting causes rod 903 to pass through the rod channel in the second alignment guide 1160 so that it is received into the coupler of the opposite screw assembly. An indicator 1195 in the handle 1196 is attached or etched to the rack to show the actuation of the rod pusher 1194. In one embodiment, when the indicator is fully extended toward the proximal end of the handle 1196 the rod pusher is fully open. When the indicator is retracted toward the distal end of the handle 1196 the rod pusher is fully actuated Once the rod is in place the rod pusher 1194 can be removed by depressing a spring loaded level that unlocks on the rack (FIGS. 71A-D). Once the release lever is depressed the rack can be retracted to pull and release the rod pusher 1194. At this point the collar 1198 can be disengaged so that the rod pusher 1194 can be removed. In some embodiments the rod introducer assembly 1186 is included with the rod pusher 1194. In this way the rod introducer assembly 1186 does not have to be removed before the rod is pivoted toward the second screw assembly. The rod pusher 1194 may also be provided with depth, tip and other markings. The markers may include, for example, visible, radiopaque, ultrasonically reflective, or magnetic markers. In some embodiments of the invention extensions and/or additional tools may be used to apply an additional mechanical advantage, such as to assist the rod in passing through tissue when the rod is pivoted. For example, a vibrational transducer may be provided which applies micro-pushes or taps to the rod.

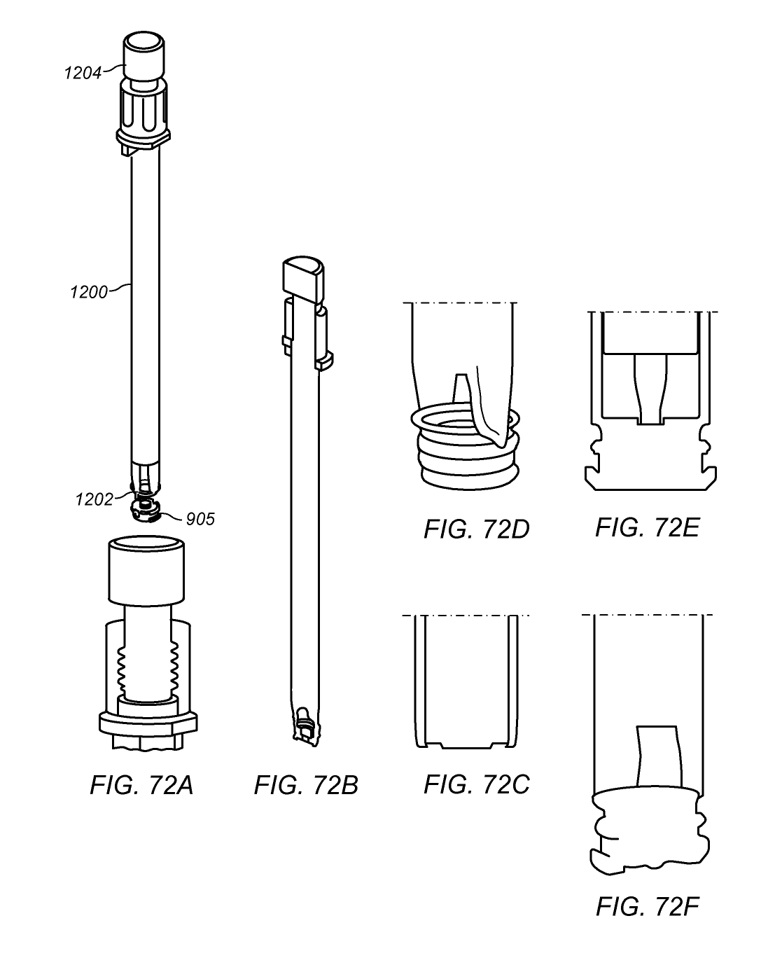

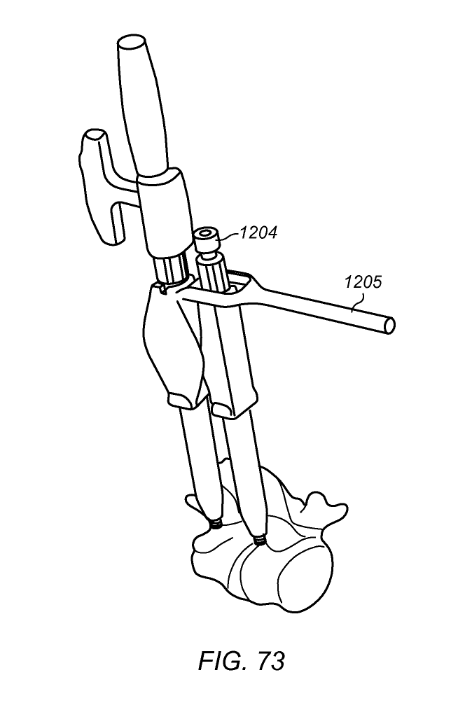

FIGS. 72A-F show a cap inserter instrument that is used to place the cap assembly 905 into the grooves of the seat 915 to secure the end of the rod. As shown, the distal end of the cap inserter 1200 has tangs 1202 that mate with recesses in the cap assembly 905 to ensure proper orientation so that the cap lugs properly engage with the mating groove in the seat 915. The tangs 1202 may be spring loaded so that they exert a force on the cap assembly 905 to retain it during the insertion. Once the lugs of the cap are in the seat the knob at the proximal end of the instrument is turned to engage the lugs into the grooves of the seat. The knob 1204 on the proximal end of the inserter may be knurled for ease in handling and it may also contain a slot for a screwdriver or the like A threaded collar 1206 fits into the top of the secondary alignment guide 1160 and must be fully secured in place to ensure that the cap assembly 905 is properly seated for engagement with the seat 915 of the screw assembly 901 Instead of a threaded collar 1206, a seating collar with a lug may be used which drops into slots across the top or proximal ends of the primary and secondary alignment guides. The collar 1206 also provides mechanical advantage to push the cap before it engages with the screw assembly 901. The cap assembly 905 is inserted with the setscrew 909 in its remote, fully-retracted position to maximize the room that is available for the rod 903. The setscrew 909 is dropped into the seat 915 of the screw assembly 901, where it engages with the grooves prior to being tightened. The knob 1204 is rotated (thereby rotating the shaft of the cap inserter instrument 1200) until the cap assembly 905 is engaged into the grooves of the seat 915, which engagement may be indicated to the operator by an audible and/or tactile click. If the cap assembly 905 does not readily engage with the seat 915 (because of tissue that may be in the way, for instance), an optional cap reducer 1205 may be employed as shown in FIG. 73. By pressing on the arm of the cap reducer 1205 while rotating knob 1204, a downward force is applied that helps to engage the cap assembly 905 with the seat 915 so that the cap assembly may advance in the grooves in the seat. In an alternative embodiment, the cap reducer 1205 is included in cap inserter 1200.

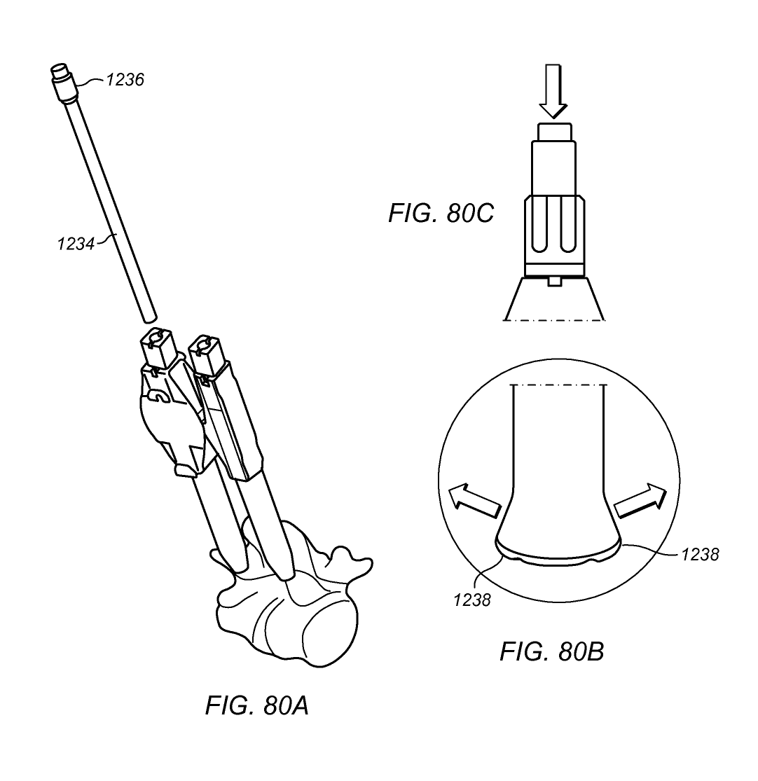

To facilitate the removal of the cap inserter instrument 1200, an optional cap release tool 1234 such as shown in FIG. 80A may be employed. The cap release tool 1234 can be inserted into the cannula of the instrument 1200. An actuator such as a button 1236 is located on the proximal end of the instrument 1234. Fins 1238 (see FIG. 80B) are located on the distal end of the instrument 1234. A plunger extends through the shaft of the instrument 1224 and is operatively coupled to the actuator 1236 and the fins 1236. When the button 1236 is actuated the fins 1238 extend radially outward. The fins 1236 exert a force on the tangs 1202 of the cap inserter instrument 1200, which spread the tangs 1202 radially outward and releases the cap inserter instrument 1200 from the cap 905 so that the cap instrument 1200 can be removed.

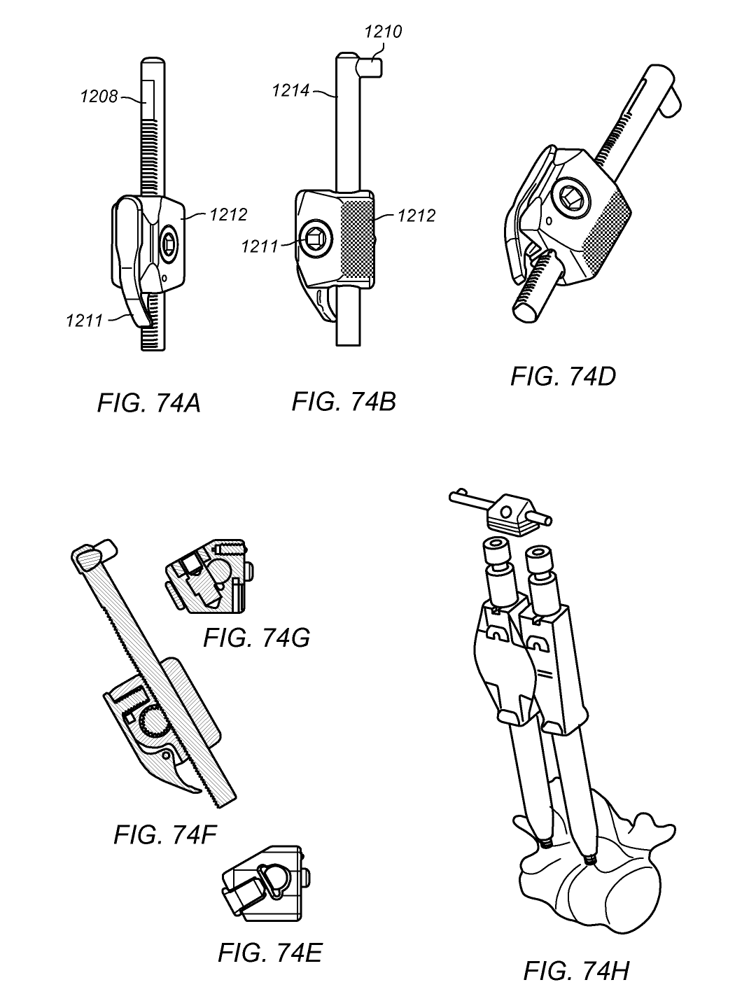

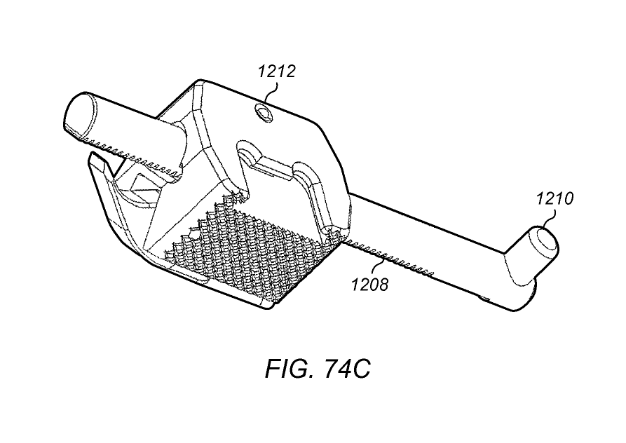

FIGS. 74A-H show a distraction/compression instrument 1208 that is used to either distract or compress the vertebra to which the bone stabilization device is attached. The distraction/compression instrument 1208 attaches to the primary or secondary alignment guides 1154 and 1160. Specifically, a recess 1209 (FIG. 74C) on the back of the distraction/compression instrument 1208 slides over and onto a corresponding mating mount on the alignment guides 1154 and 1160. A ball detent device provides just enough force or resistance to keep the instrument 1208 from coming off. That is, the distraction/compression instrument 1208 is fixedly attached to one of the alignment guides at 1154 and/or 1160. When attached to one of the guides and actuated, the instrument 1208 can pull the other guide around the pivot point (i.e., the hook and cross pin) via a lateral post 1210 when the rack and pinion are actuated. Alternatively, the instrument 1208 can be pivotally attached to both alignment guides 1154 and 1160, or even integrally formed with either or both of the alignment guides 1154 and 1106. The instrument 1208 includes a rack and pinion 1212 or other linear drive mechanism that is translatable along a rack 1214. Of course, other types of drive mechanisms may be employed such as hydraulic/pneumatic or magnetic drives, jack screw drives and rotary gears, for example. The rack 1214 then pulls the opposite alignment guide in such a way around the pivot point formed by the hook and cross pin to either distract or compress the vertebra. Depending on whether the distraction/compression instrument 1208 is mounted above the pivot point or below the pivot point determines whether distraction or compression is performed

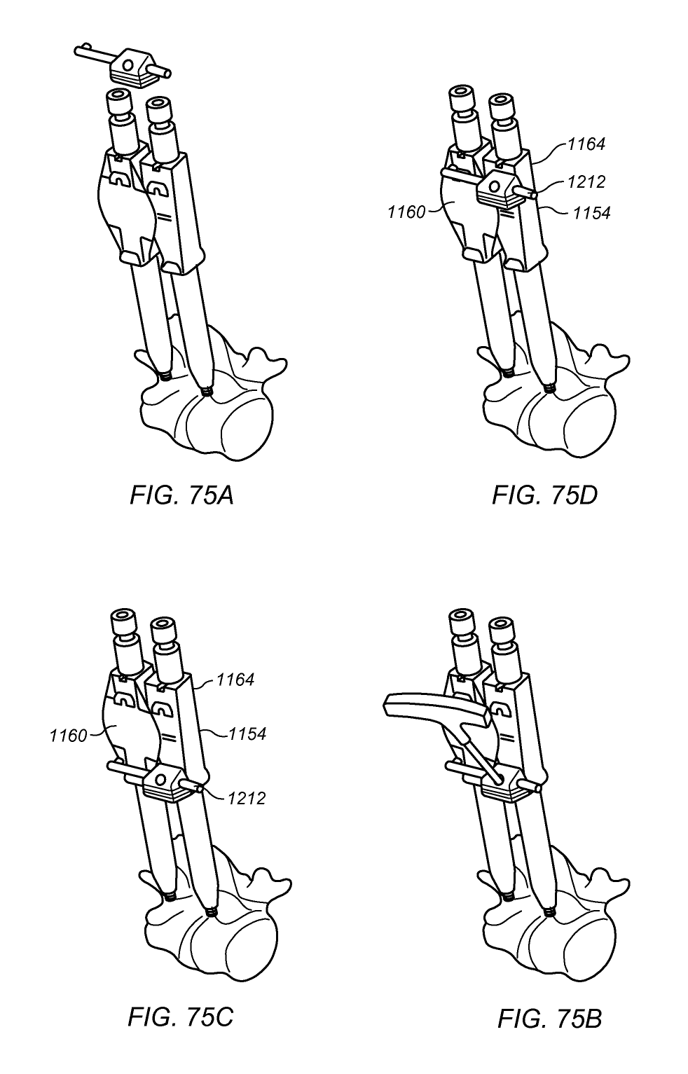

As shown in FIGS. 75A-D, the instrument 1208 is attached at a location above the pivot point formed by the primary and secondary alignment guides 1154 and 1160 when it is used to distract the vertebra (by pulling together the rack and pinion 1212) or compress the vertebra (by pushing apart the rack and pinion 1212) Likewise, as shown in FIG. 75B, the instrument 1208 is attached at a location below the pivot point formed by the primary and secondary alignment guides 1154 and 1160 when it is used to contract the verebra (by pulling together the rack and pinion 1212) or distract the verebra (by pushing apart the rack and pinion 1212). The linear drive mechanism 1212 includes an adjustment screw 1216 to extend or retract the rack 1214. Rotation of the screw 1216 with the screwdriver in one direction causes distraction and rotation in the opposite direction causes compression. By extending or retracting the rack 1214 in this way a force is applied between the primary and secondary alignment guides 1154 and 1160. A linear or rotary encoder, or a force measuring transducer, may be provided to increase the precision of the force that is applied and/or the actual measurement of the distraction or compression that is achieved. The force is translated through the STAs 130 to the screw assemblies 901, which then impart the force to extend or retract the vertebra to restore disc height to the degenerated or collapsed disc. Once the desired degree of compression or distraction is achieved, the setscrew 1216 of the cap assembly is tightened down on the rod to secure the relative position of the screw assemblies 901. A spring loaded lever 1211 serves as a lock and release mechanism on the distraction/compression instrument. The lever 1211 engages with the drive mechanism 1212 so that it can slide to release the pressure so that the instrument 1208 can be removed. In some embodiments of the invention the instrument 1208 may also exert a force directly on the STAs 1130 by gripping each STA 1130 and applying a relative torsional forces between them. For instance, the instrument 1208 may include its own hinge portion in addition to the linear drive mechanism.

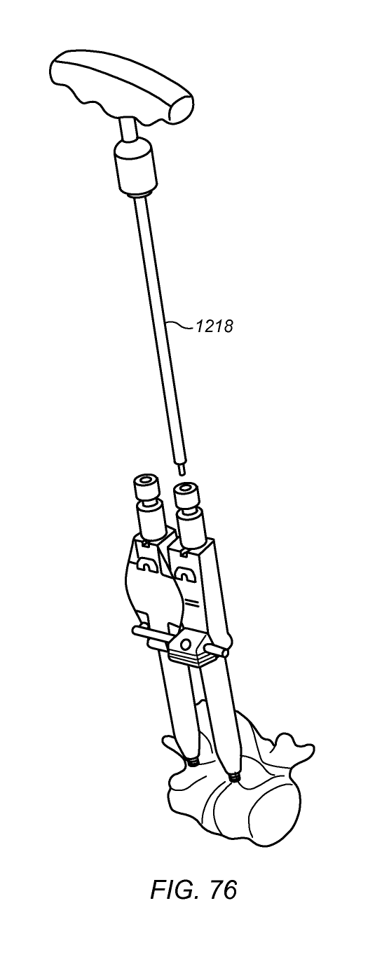

FIG. 76 shows a torque indicating driver 1218 that is used to tighten the setscrew 909 in the cap assembly 905 while the distraction/compression instrument 1208 is still in place. The shaft of the torque indicating driver 1218 is configured so that it can be inserted through the cannulae of the primary and secondary alignment guides 1154 and 1160 and engage with the setscrews 909. One setscrew 909 is first provisionally tightened and then the other setscrew 909 is fully tightened. The torque indicating driver 1218 includes a torque measurement gauge or strain gauge to tighten the setscrews 909 to the desired torque. Alternatively, the driver 1218 may be configured to strip or shear at a known torque so that a safety threshold is provided to prevent excessive forces from being applied to the implanted components and/or the patient. After the second setscrew 909 is fully tightened, the first setscrew 909 is then fully tightened to the desired torque.

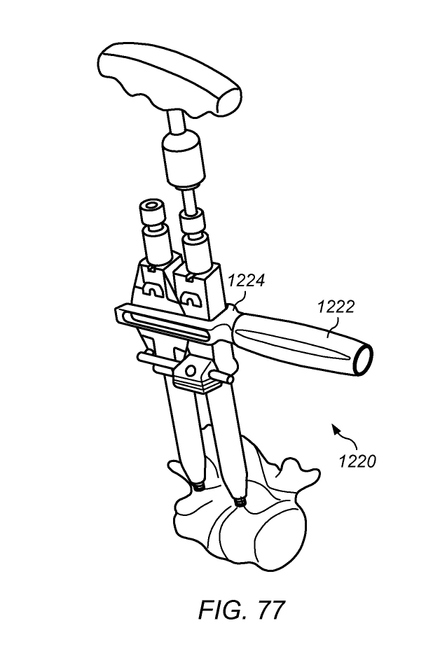

In some cases a torque stabilizer may be used to provide a counter torque to reduce or prevent undue stress from being placed on the construct (implants and vertebral bodies, etc.) such as during final tightening of the setscrews with the torque indicating driver 1218. As shown in FIG. 77, torque stabilizer 1220 attaches to the primary and/or second alignment guides 1154 and 1160 so that the operator can stabilize the system during the final tightening procedure. The torque stabilizer 1220 includes a handle 1222 from which extends a fork that slides over a corresponding lug on the primary and secondary alignment guides 1154 and 1160. In an alternative embodiment, the torque stabilizer may be included in primary and/or secondary alignment guides 1154 and 1160 such that a stabilizing force can be applied at any time without the need to attach a separate tool. In some cases the torque stabilizer also may be used to apply a force to one or more of the dilators (e.g., the largest diameter dilator) to advance the dilator as it is inserted through tissue. To accomplish this, a dilator insert is press fit into the end of the torque stabilizer 1220. The insert slips over the diameter of the dilator and advances to its end top surface.

The torque stabilizer handle provides a grip to help apply force to the proximal end of the dilator, such as to advance the dilator through tissue when significant resistance is met.

The torque stabilizer 1220 may include a lumen to accommodate a guidewire, thereby allowing over-the-wire placement when force is exerted on the proximal end of the dilator.

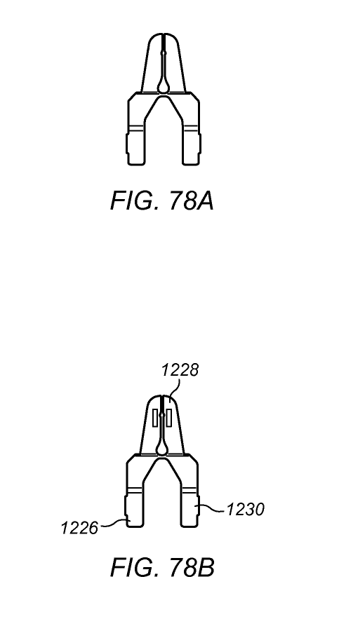

FIGS. 78A-B show a guidewire clip 1226 that may be used to prevent the guidewire from inadvertently advancing during the procedure. If the guidewire were to improperly advance it could perforate through the anterior vertebral wall.

The guidewire could also puncture one of the major arteries along the anterior column of the spine. The clip 1226 may also serve as a visual reference to the operator that indicates if there is any movement of the guidewire, either forward or backward, during the procedure. In some embodiments the guidewire clip 1226 may include a slip sensor 1228 that is operatively coupled to alarm transducer 1230. If the guidewire should slip out of the clip 1226, the slip sensor 1228 will activate the alarm transducer 1230 to inform the operator.

Many of the tools described above include one or more engagement means such as matched sets of internal and external threads. Of course, various other types of engagement means may be employed instead, such as press-fits, frictional fits (e.g., tapered fits), bayonet locks and the like. Since a downward force is often applied to the tools (including the engagement means), the tools should be configured to provide a significant mechanical advantage so that a large force can be generated, while allowing the operator to precisely control the force and the distance over which the force is applied. Although it has only been specifically noted with respect to some of the tools described above, any or all of the tools may include markers, which may be visible either with or without equipment. The markers may be used for a variety of purposes, such as to facilitate rotational alignment or orientation (within a single tool, between different tools, and/or between one or more tools and the patient's spine), to measure insertion depth or rod length, to determine engagement or deployment status, or any combination thereof.

The previously described tools can be used to operatively implant the bone stabilization device 100. One illustrative procedure using such tools to implant the device will now be presented below.

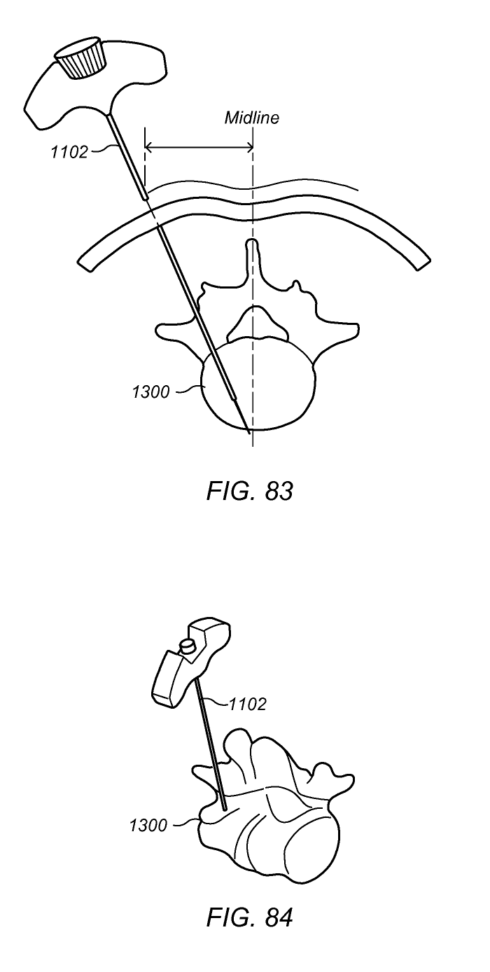

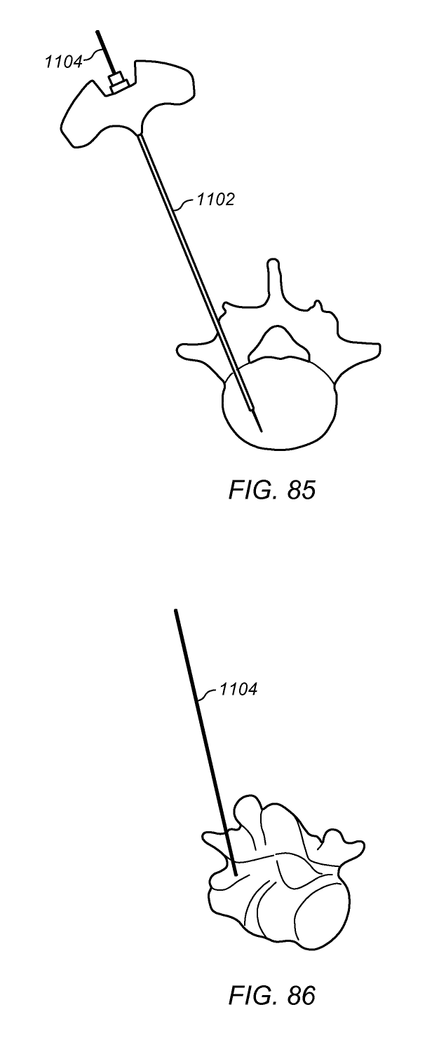

As shown in FIG. 83 the surgical procedure begins by gaining access to the pedicle 1300 using the target needle 1102 under fluoroscopy. The entry point is generally 3-4 cm lateral of the midline of the spine. The target needle is inserted about two-thirds of the way through the vertebral body while avoiding penetration of the anterior wall. The target needle 1102 is carefully removed (FIG. 84) while leaving the guide in place. Next, in FIG. 85 the guidewire 1104 is inserted through the guide. The distal end of the guidewire 1104 extends into vertebral body, about 10 mm from the anterior wall. The proximal end of the guidewire 1104 resides outside the patient so that it can accept over-the-wire devices.

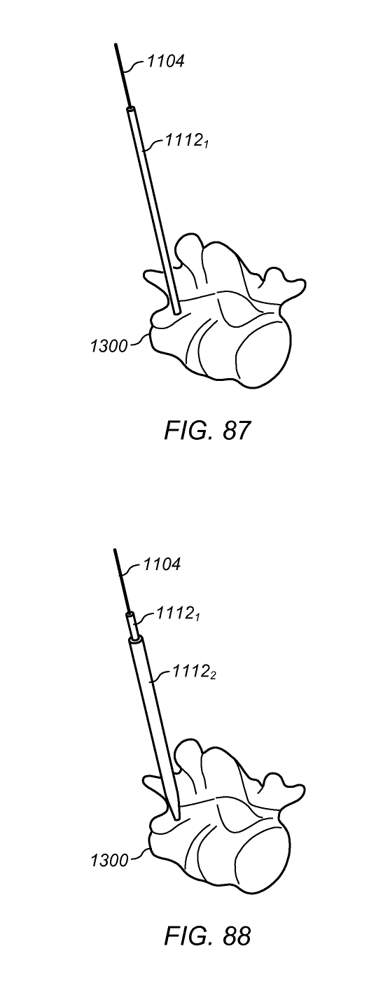

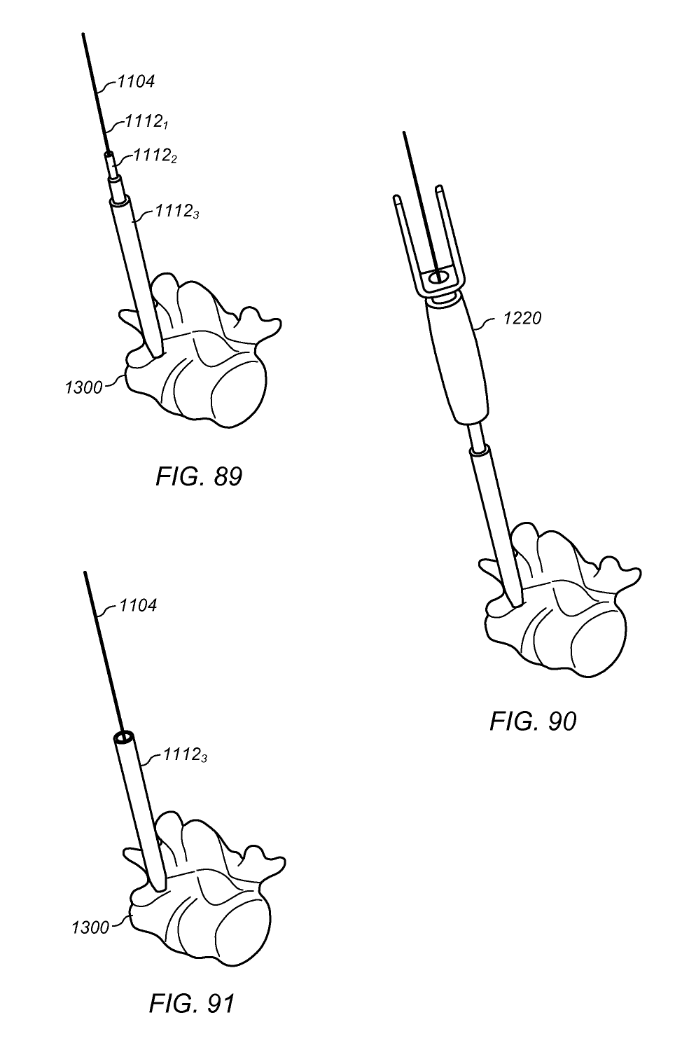

An over-the-wire “exchange” is shown in FIG. 86 in which the guide is removed, leaving the guidewire 1104 in place. Tissue dilation is next performed (FIG. 87) by placing the first of a series of dilators over-the-wire, starting with the smallest diameter dilator 1112₁, to expand/dilate the tissue residing between the entry site and the pedicle 1300 so that a safe pathway can be provided for inserting instruments and implants to the surgical site. As shown in FIGS. 88-89, the second dilator 1112₂ is placed over the first dilator 1112₁, and the third dilator 1112₃ is placed over the second dilator 1112₂. In some cases the torque stabilizer 1220 may be placed over-the-wire and used to exert force on the dilator (FIG. 90). The tip of the final dilator (e.g., dilator 1112₃) may have “teeth” to exert a force that grips the pedicle 1300, which can be helpful during the tapping and screw insertion steps so that there is no slippage or the like. The dilator may be manipulated (e.g. back-forth rotation) to enhance this grip force. As previously noted, a single expandable dilator (e.g., a rolled tube that unfolds to expand) may be used instead of the series of dilators. The tissue dilation steps are completed by removing all but the largest diameter dilator by an over-the-wire exchange, leaving only the largest diameter dilator in place (FIG. 91).

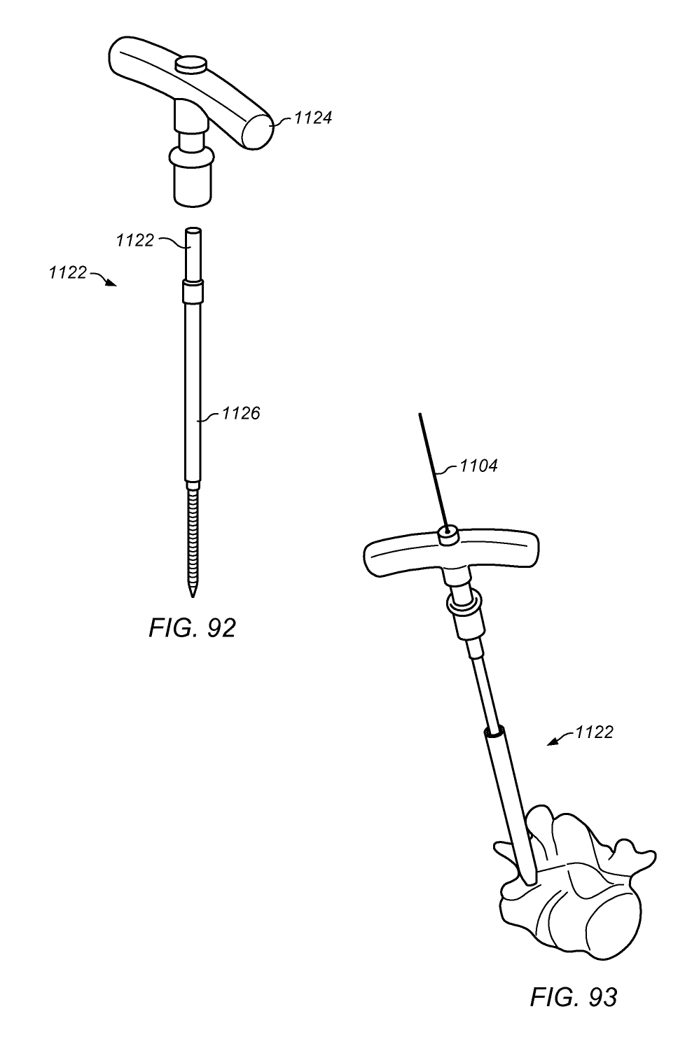

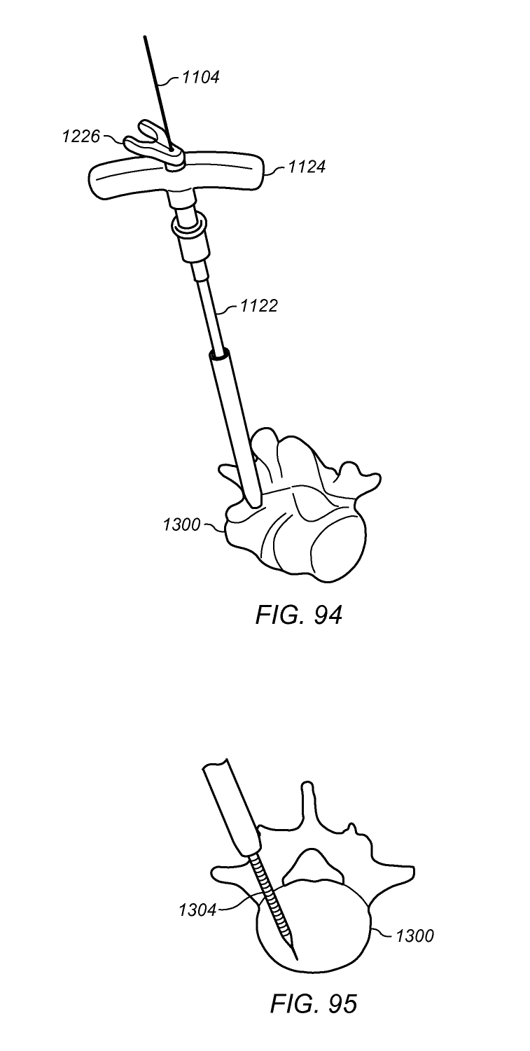

As shown in FIG. 92, the tap device 1122 is assembled by snap fitting any one of the handles 1124 onto the tap drive 1126 of the appropriate size. The tap device 1122 is placed over-the-wire and through the largest diameter dilator 1112₃ and extends up to the pedicle surface (FIG. 93). Optionally, as shown in FIG. 94, the guidewire clip 1226 may be attached to the guidewire 1104 to maintain the guidewire's position. In this case the handle of the tap device 1122 provides a visual reference during the tapping process to prevent inadvertent advancement of the guidewire 1104, thereby avoiding penetration of the vertebral body. The guidewire clip 1226, in addition to or instead of being integral to the tap as previously described, may be integral to the dilator 1112₃. The tapped hole 1304 that is created by rotating the handle 1124 under fluoroscopy is shown in FIG. 95. At this stage the guidewire 1104 should be visually checked to ensure that it has not advanced. If the guidewire clip 1126 is present, the distance between it and the point to which the handle 1124 is advanced is indicative of the screw length that is needed. The guidewire clip 126, if present, may also be incrementally advanced to prevent undesired guidewire advancement. As indicated in FIG. 95, the distal end of the tap generally should be advanced to within about 10-15 mm of the distal end of the guidewire 1104, as can be seen under fluoroscopy.

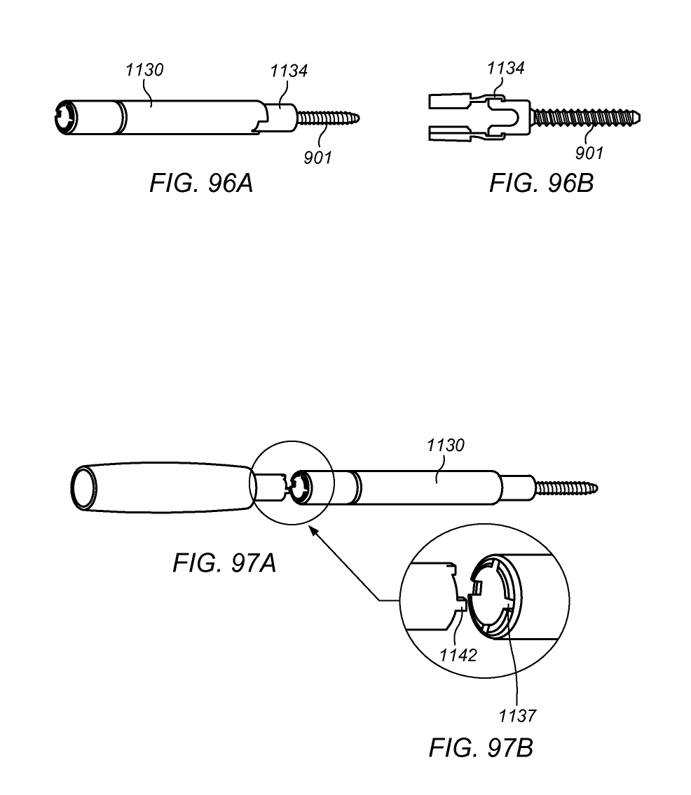

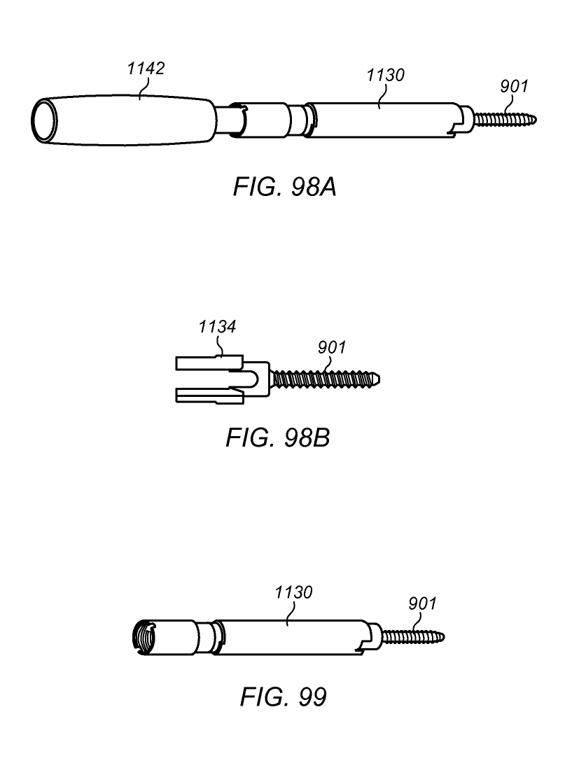

The procedure continues by attaching the STA 1130 to the screw assembly 901 while the STA 1130 is in its open or advanced position (See FIGS. 96A-B). Next, as indicated in FIGS. 97A-B, the locking tool 1142 is connected to the STA 1130 by engaging the tangs 1144 of the locking tool 1142 with the notches 1137 of the STA 1130. The screw assembly 901 is locked to the STA 1130 by rotating the locking tool 1142 until the tangs 1134 of the STA 1130 are closed or retracted (FIGS. 98A-B). The locking tool 1142 engages with the bushings of the STA 1130 so that rotation of the locking tool 1142 causes the tangs to retract. Once the screw assembly 901 is properly engaged with the STA 1130 the locking tool is removed (FIG. 99).

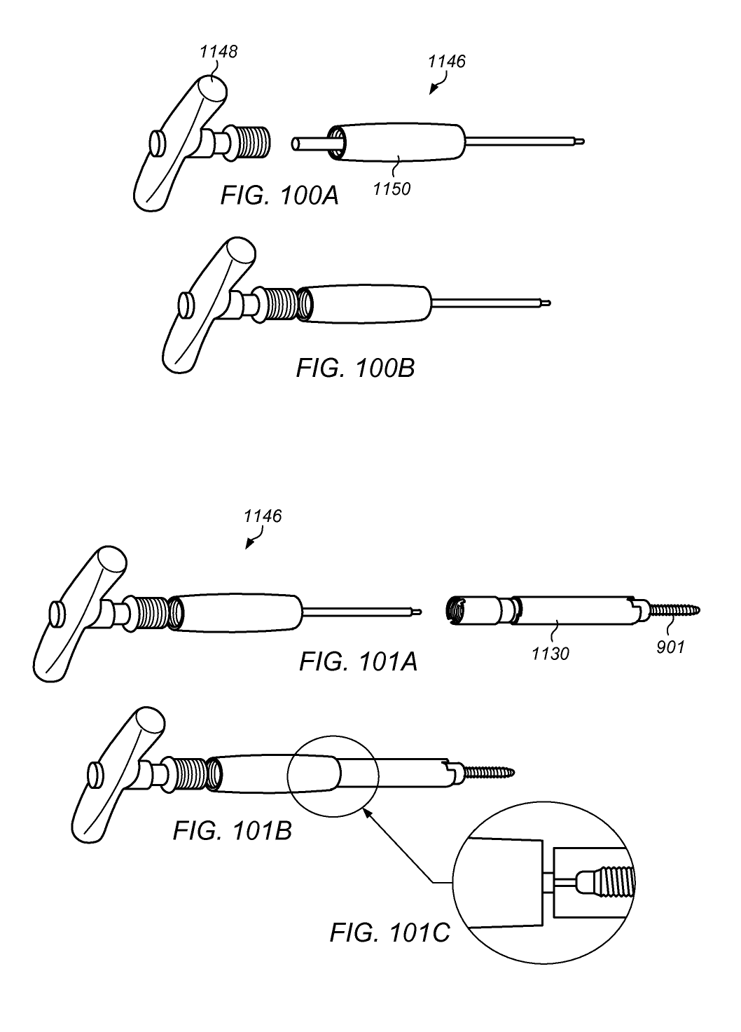

The polyaxial screwdriver 1146 is assembled by attaching the handle 1148 to the tubular body 1150 (FIGS. 100A-B) and the screwdriver 1146 is in turn attached to STA 1130 by passing the body 1150 though the proximal opening in the STA 1130 (FIGS. 101A-C). The hexagonal end of the screwdriver 1146 engages with the hexagonal opening in the spherical head 919 of the screw 911.

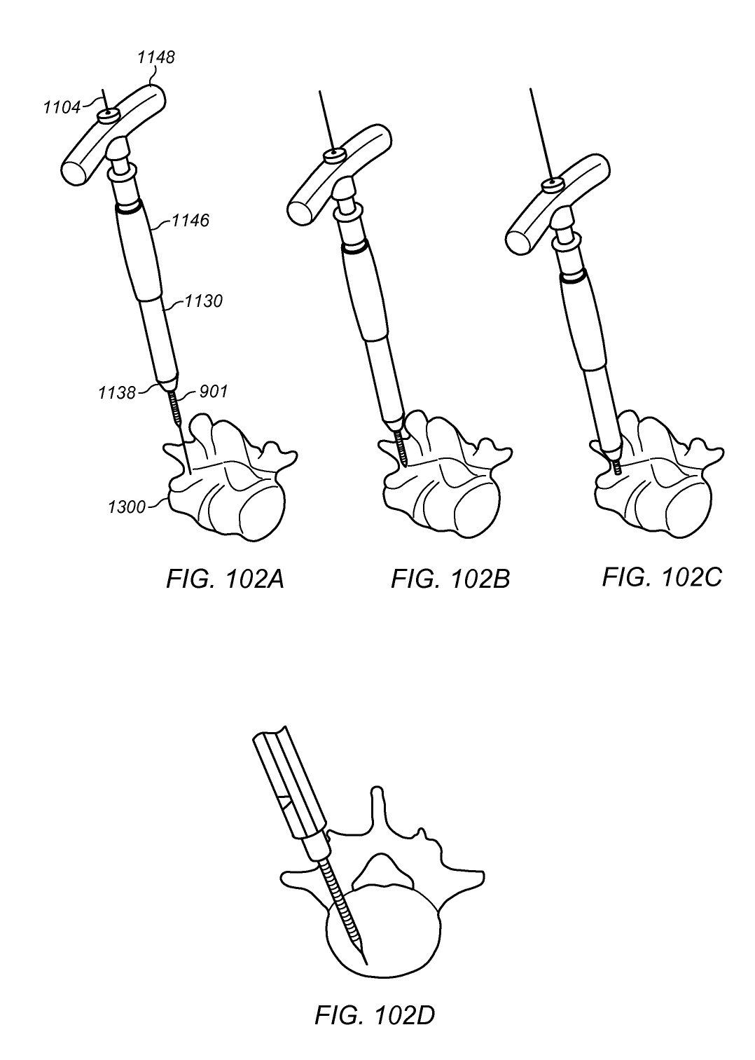

Next, the screw assembly 901, STA 1130 and screwdriver 1146 are inserted over the wire into the pedicle. As shown in FIGS. 102A-D, this is accomplished by placing the guidewire 1104 through the cannulas of the screw assembly 901, STA 1130 and screwdriver 1146. During this process the operator should hold the STA 1130 to prevent the screwdriver 1146 from disengaging. Alternatively, the screwdriver 1146 and STA 1130 may have locking collars so that it is not necessary to hold the STA 1130. Such locking collars may also facilitate transmission of torsional forces. At this point the lugs of the screwdriver 1146 should be fully engaged with the notch on the STA 1130 to ensure that torsional forces will be transmitted from the screwdriver 1146 to the screw assembly 901. The operator then rotates the handle 1148 while holding the mid-point of the tubular body 1150 to drive the screw assembly 901 to the appropriate depth. The screw assembly 901 should not be advanced so far that the seat 915 contacts the pedicle 1300. In this way the seat 915 has sufficient freedom of movement to allow self-alignment with the rod 903 when the rod 903 is inserted. During insertion of the screw assembly 901, as well as during the remaining steps of the procedure, it is important that the orientation of rod channel 1138 of the STA 1130 be maintained in the cephalad-caudal direction so that the screw assembly 901 will be properly aligned with the subsequently installed second screw assembly, thereby allowing the rod 903 to be properly connected to both screw assemblies. Proper alignment can generally be verified under fluoroscopy using any of the various markings or indicators located on the STA 1130 and/or on the instruments inserted into the STA 1130. Once the screw assembly 901 is installed, the screwdriver 1146 and the guidewire 1104 are removed.

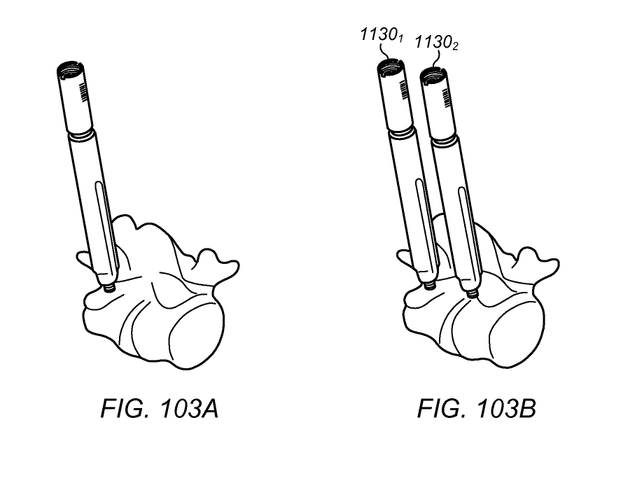

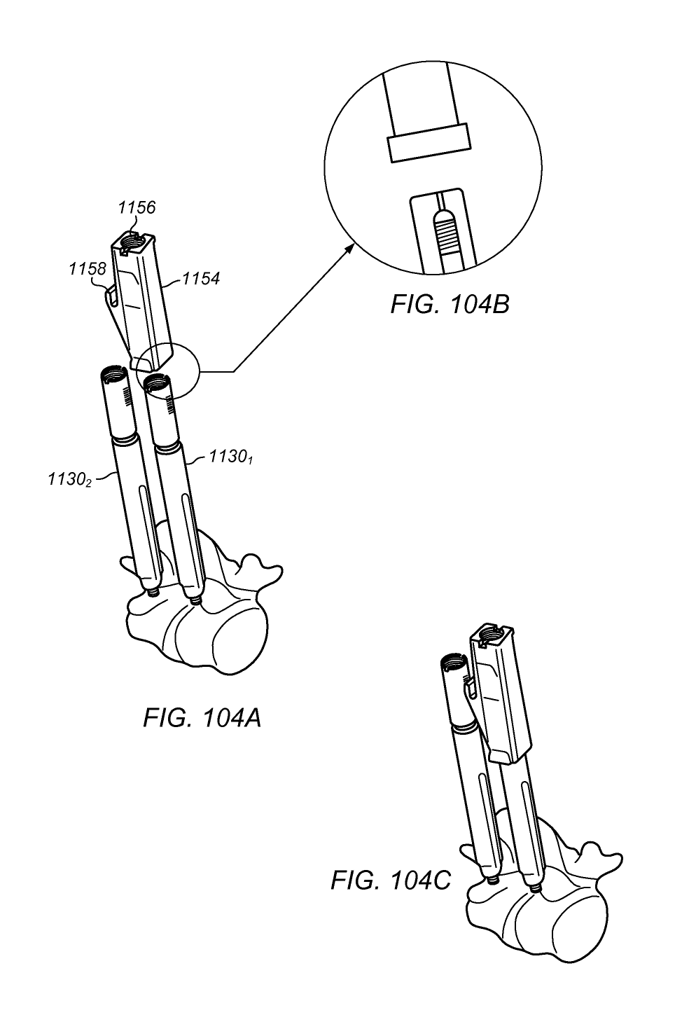

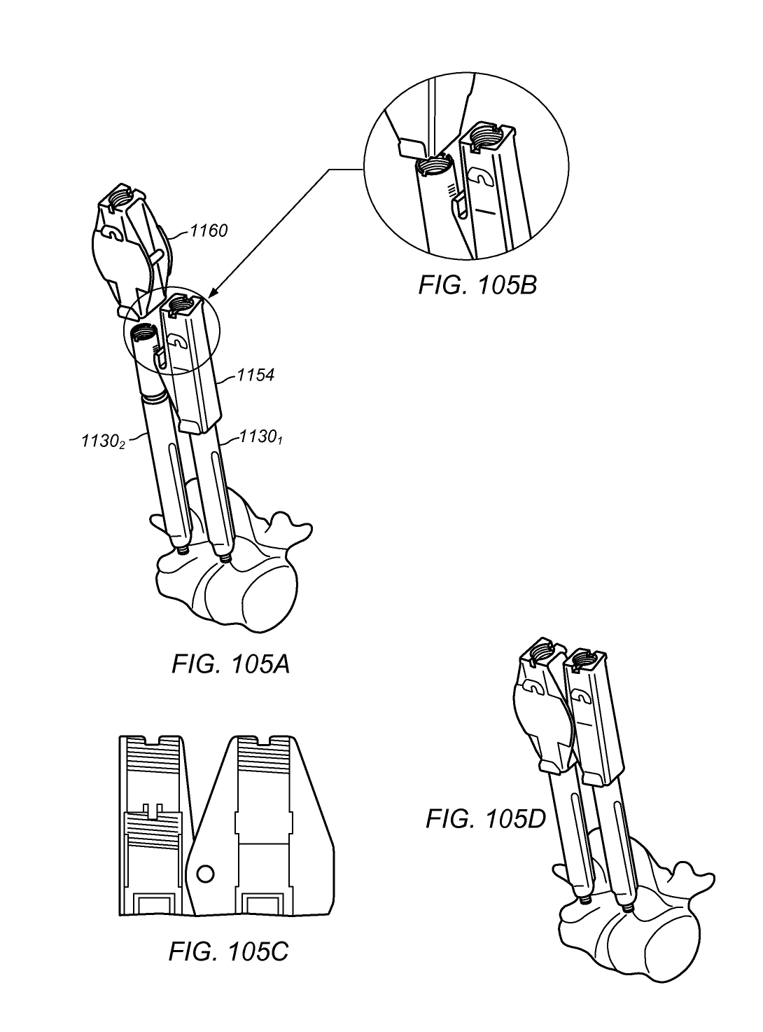

The previously described steps are repeated for the adjacent vertebra pedicle (or in some cases a non-adjacent vertebra pedicle) to install the second screw assembly. The first and second STAs 1130₁ and 1130₂ are shown in FIGS. 103A-B after the screwdriver 1146 is removed.

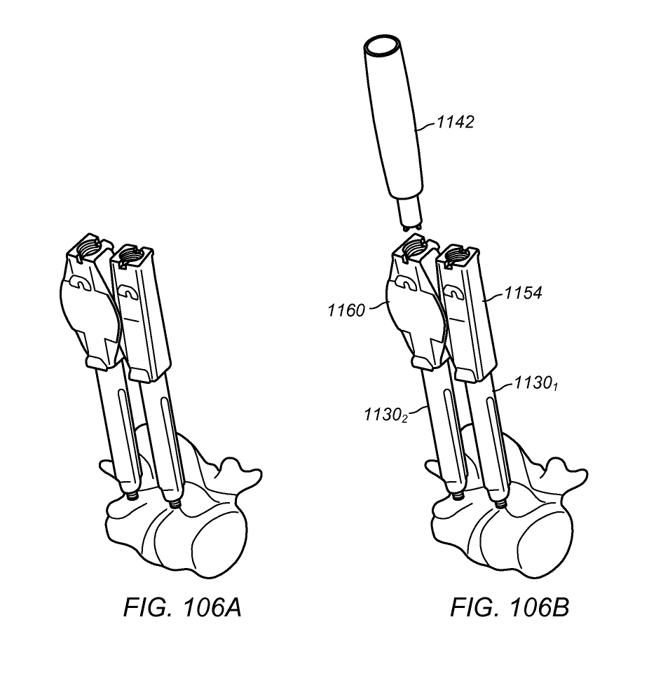

After both screw assemblies have been installed the primary alignment guide (PAG) 1154 is placed over the first STA 1130₁ so that it is slidingly received by the proximal end of the first STA 1130₁ (FIGS. 104A-C). The markings or other indicators on the PAG 1154 should be properly aligned with the marking on the first STA 11301 so that the seats 915 and couplers 913 of the screw assemblies 901 are correctly aligned to receive the rod 903. Similarly, as shown in FIGS. 105A-D, the secondary alignment guide (SAG) 1160 is placed over the second STA 1130₂ so that it is slidingly received by the proximal end of the second STA 11302. At this point the cross pin 1164 of the SAG 1160 drops over the hook 1158 of the PAG 1154 to create a hinge. Once the cross pin 1164 and the hook 1158 are engaged, the locking tool 1142 is attached to the SAG 1160 by engaging the tangs 1144 with the notches in the bushings of the SAG 1160 (FIGS. 106A-B). The locking tool 1142 is rotated by the operator so that the SAG 1160 is locked to the STA 1130₂.

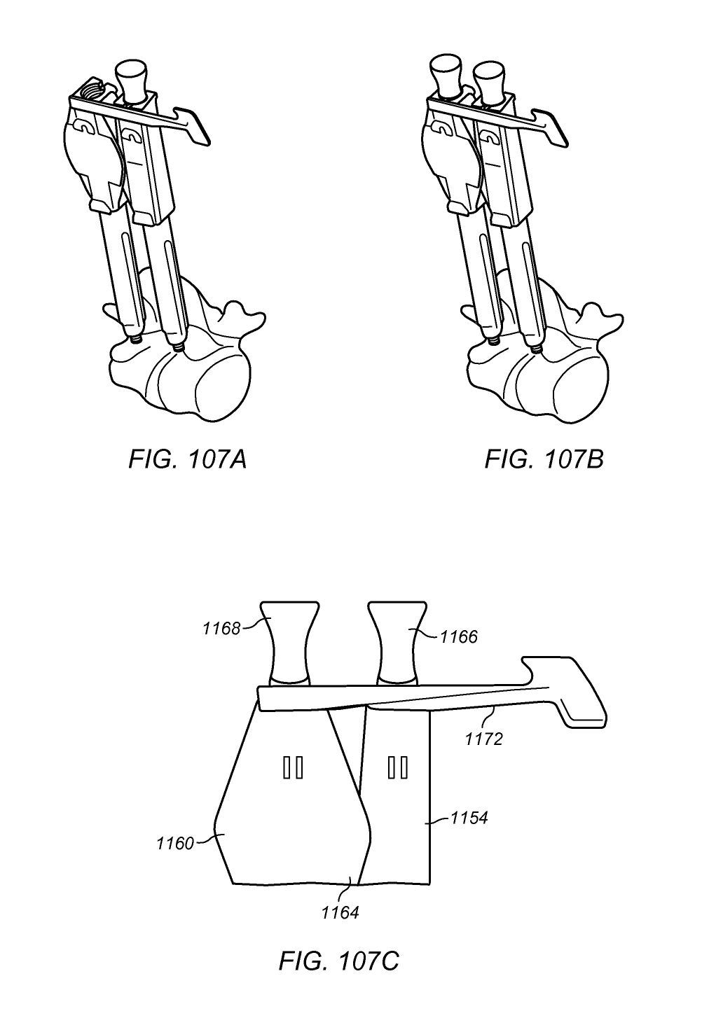

Next, to determine the proper rod length that is to be used, the rod gauge indicator 1168 is attached to secondary alignment guide 1160 and the rod gauge measurement device 1166 is attached to the primary alignment guide 1154 (FIGS. 107A-C). The screw length can be directly read off the scale of the rod gauge measurement device 1166. It will generally be sufficient to round up the rod length to the nearest whole value indicated on the scale. As previously noted the rod gauge indicator 1168 (or another tool that measures the angle of the hinge 1164) may be integrally formed with the SAG 1160 and the rod gauge measurement device 1166 (or another tool that measures the angle of the hinge 1164) may be integrally formed with the PAG 1154, thereby avoiding the need to separately insert these two instruments. In some cases the rod length measuring tool may not even be used. Instead, the appropriate rod length can be determined simply using fluoroscopy.

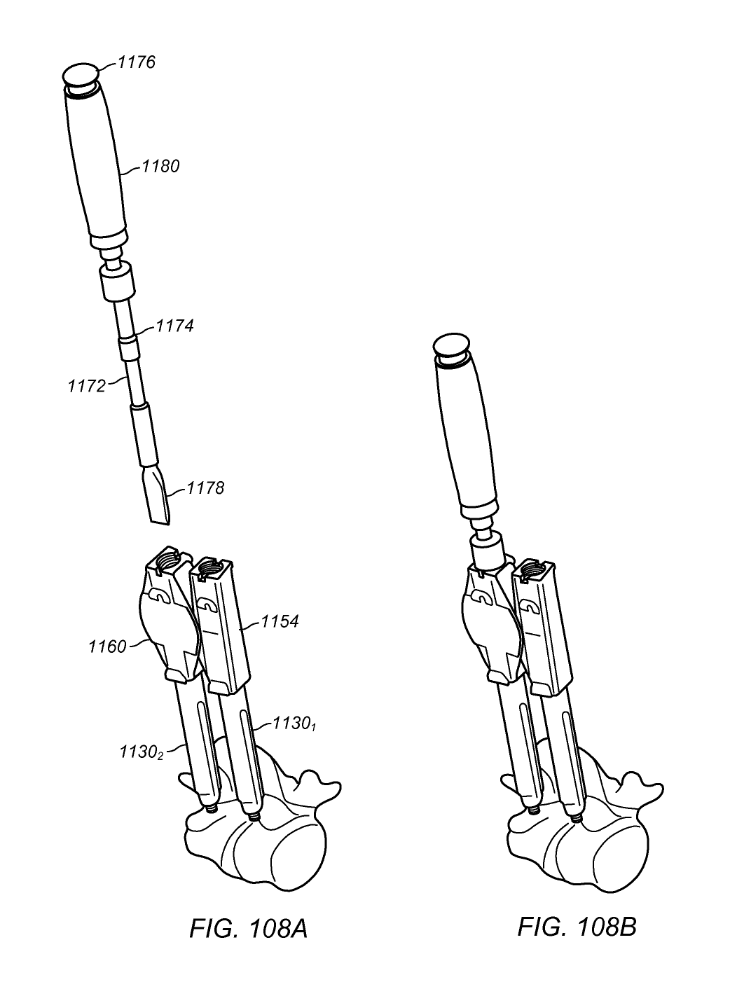

In preparation for inserting the rod 903, In FIGS. 108A-B the tissue splitter 1174 is inserted into and properly aligned with the PAG 1154 and/or the SAG 1160. The tissue splitter 1174 is only used when tissue separation is needed. The collar 1184 of the tissue splitter 1174 is rotated so that it engages with the threads of the PAG 1154 and/or the SAG 1160. The blade 1178 is deployed by depressing the button 1176 on handle 1180. In this way the tissue is dissected between the seats 915 of the screw assemblies 901. To facilitate dissection, the tissue splitter may be energy assisted. In some embodiments the deployed blade is used to measure the proposed screw length under fluoroscopy. For these purposes, the blade may include radiopaque markers or holes indicative of the desired rod length. Instead of using a dedicated tissue splitter tool, tissue separation may be accomplished by other means. For example, the rod 903 may have a sharpened surface that dissects the tissue while it is being pivoted into position and/or energy may be delivered to cut or ablate tissue.

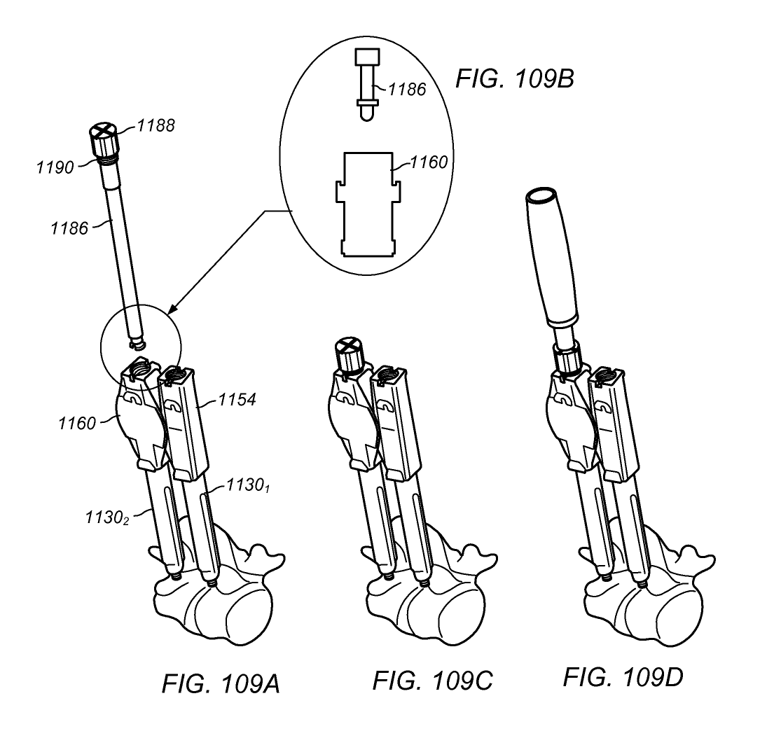

After the appropriate length rod 903 is selected based on the information obtained from the rod length measuring tool and/or other means, the rod 903 is attached to the rod introducer assembly 1186 as previously shown in FIG. 70. Next, as shown in FIGS. 109A-D, the rod 903 is inserted into the PAG 1154 or the SAG 1160 and properly aligned using any of the alignment mechanisms that are provided. The rod 903 is advanced through the PAG 1154 or SAG 1160 until the base 921 of the rod 903 engages with the seat 915 and coupler 913 of the screw assembly 901. The collar 1188 is rotated to push the rod 903 into its proper position. If needed, the locking tool 1142 may be used to help rotate the collar 1188. Once the rod is properly positioned and it has been confirmed that the rod 903 is properly secured to the seat 915 and the coupler 913, the rod introducer 1186 is removed.

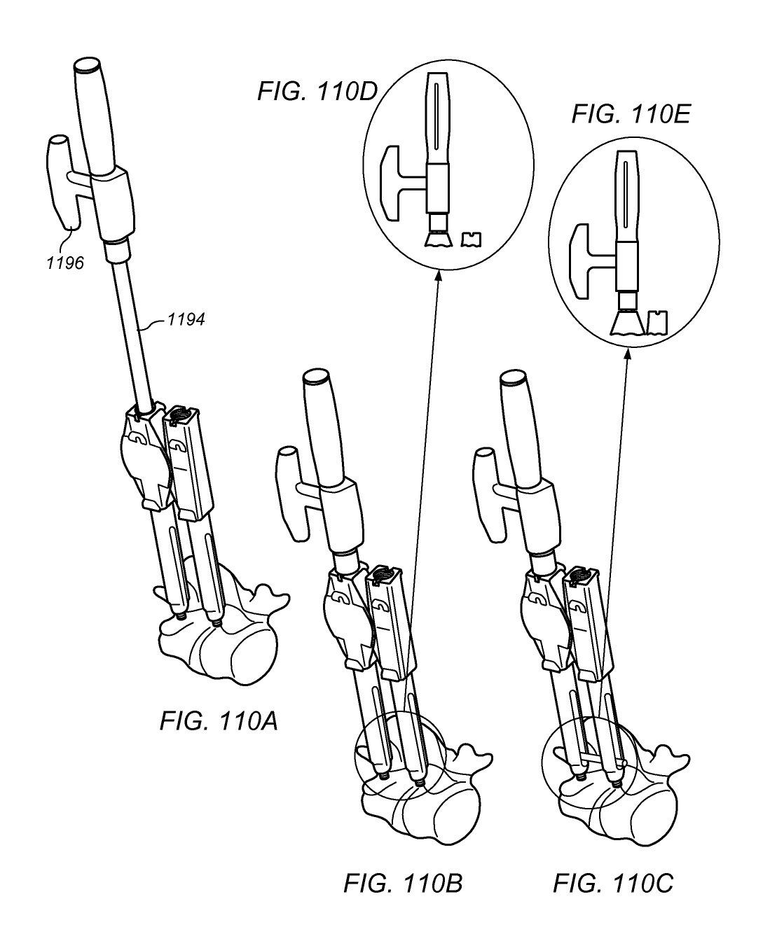

The rod 903 is next pivoted into position using the rod pusher 1194. The rod pusher 1196 is inserted into the cannula of the PAG 1154 (or the SAG 1160 if the rod 903 was inserted therethrough) and properly aligned using any of the alignment mechanisms that are provided (FIGS. 110A-E). Once properly engaged with the PAG 1154, the handle 1196 is rotated to advance the piston and apply force onto the rod 903 so that it pivots toward the second screw assembly. The rod pusher 1194 is then removed.

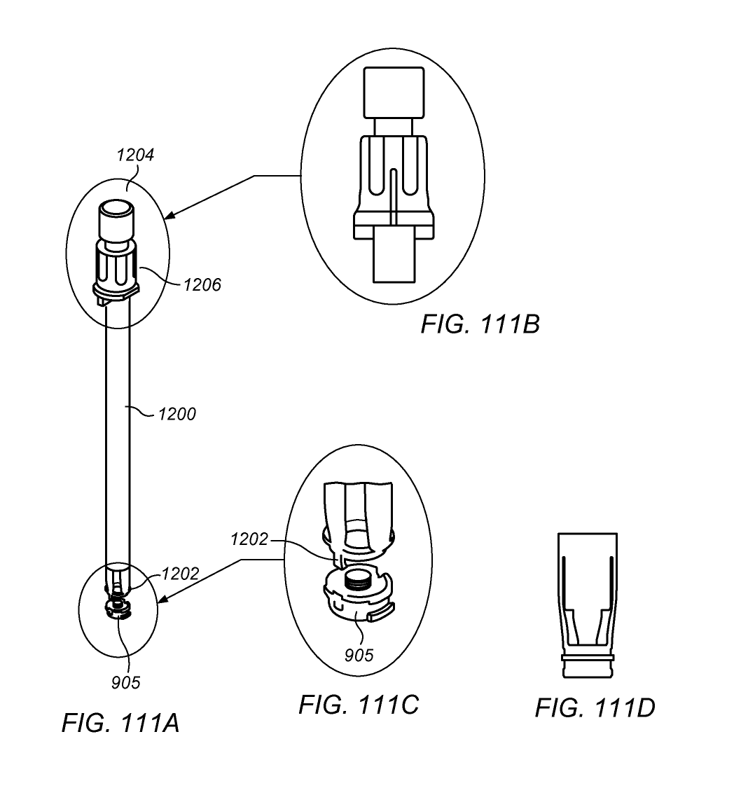

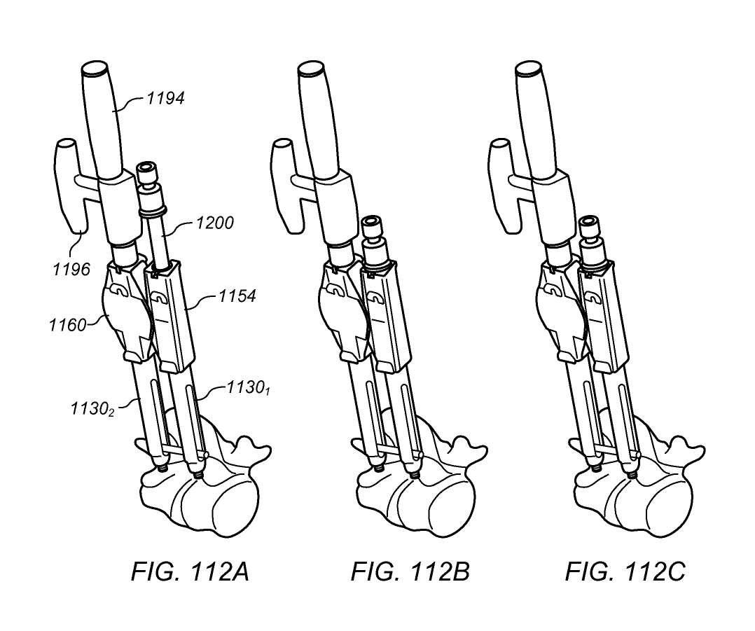

After the rod is in place, the cap inserter instrument 1200 is used to place the cap assembly 905 over the end of the rod and fit it into the grooves of the seat 915. As shown in FIGS. 111A-D, the tangs 1202 mate with recesses in the cap assembly 905 to ensure proper orientation so that the cap lugs properly engage with the mating groove in the seat 915. The threaded collar 1206 of the cap inserter instrument 1200 is advanced through the primary alignment guide 1154 and secured in place (FIGS. 112A-C). It should be confirmed that the cap assembly 905 is inserted with the setscrew 909 in its remote, fully-retracted position to maximize the room that is available for the rod 903. The setscrew 909 is oriented with the lugs in position to be dropped into the seat 915 of the screw assembly 901, where it engages with the grooves prior to being tightened. The knob 1204 is rotated (thereby rotating the shaft of the cap inserter instrument 1200) until the cap assembly 905 is engaged into the grooves of the seat 915, which engagement may be indicated to the operator by an audible and/or tactile click. If the cap assembly 905 does not readily engage with the seat 915 (because of tissue that may be in the way, for instance), the optional cap reducer 1205 may be employed as shown in FIG. 73. By pressing on the arm of the cap reducer 1205 while rotating knob 1204, a downward force is applied that helps to engage the cap assembly 905 with the seat 915 so that the cap assembly may advance in the seat threads. In an alternative embodiment, the cap reducer 1205 is included in cap inserter 1200.

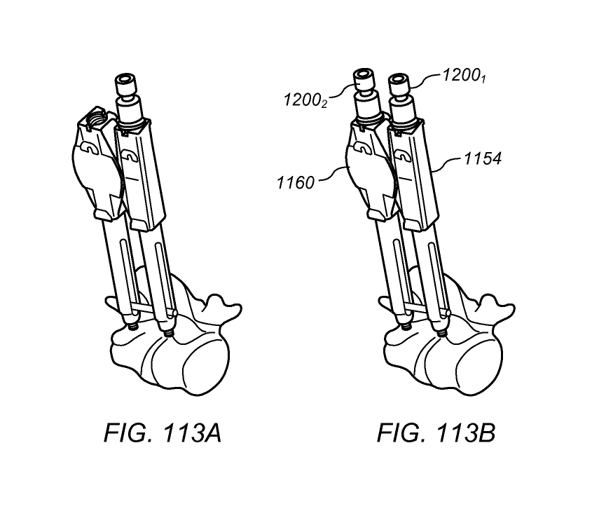

A second cap inserter instrument 1200 is used to install a second cap assembly 905 through the SAG 1160 in a process similar to that used to insert the previous cap assembly through the PAG 1154. FIGS. 113A-B shows both the first and second cap inserter instruments 1200₁ and 1200₂ in the PAG 1154 and SAG 1160, respectively.

Next, the distraction/compression instrument 1208 is attached to the primary and secondary alignment guides 1154 and 1160 in the manner discussed above in connection with FIGS. 74A-B so that the vertebra can be either distracted or compressed by an appropriate amount. Finally, the torque indicating driver 1218 is used to tighten the setscrews 909 in the two cap assemblies 905 while the distraction/compression instrument 1208 is in place. If needed, the torque stabilizer 1220 may be used to facilitate the process. In general, a mechanical advantage is achieved by placing the instrument 1208 above the hinge formed by the cross pin 1164 and hook 1158 since large forces can be generated. On the other hand, if the instrument 1208 is placed below the hinge, finer control and precision can be achieved.

Finally, the bone stabilization device installation process is completed by removing the various instruments. First, the cap inserter instruments 1200₁ and 1200₂ are removed. If needed, the cap remover instrument 1234 shown in FIGS. 80A-C may be used to assist in the removal of the cap inserter instruments 1200₁ and 1200₂. Next, the locking tool 1148 is used to disengage the PAG 1154 and 1160 from the STAs 1130. Once the STAs are loosened by the locking tool 1148 they can be removed by gripping them at their knurled ends.

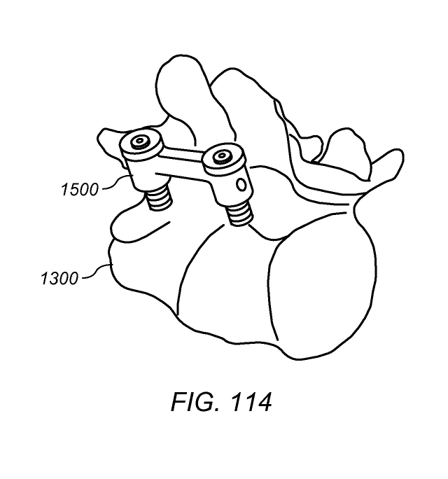

FIGS. 113A-B shows the bone stabilization device 1500 installed in one side of the vertebral segment. A second bone stabilization device will generally be installed on the other side of the spine to achieve bilateral bone stabilization. The second bone stabilization device can be installed by the same procedure presented above. FIG. 114 shows both bone stabilization devices 1500₁ and 1500₂ installed in the vertebra. Some or all of the tools presented above may be suitably modified to achieve simultaneous or partial simultaneous bilateral construction by simultaneously installing some or all of the components of the two bone stabilization devices. (repeating one or more steps and/or reversing one or more steps, for example: remove/replace pedicle screw <e.g. with larger one>, pivoting rod back up <e.g. to reorient spinal alignment which may require additional tissue dissection>, remove/replace rod <e.g. with longer or shorter rod>, remove an existing system of the present invention <e.g. with similar tools or in an open procedure>, etc.)

Although various embodiments are specifically illustrated and described herein, it will be appreciated that modifications and variations of the present invention are covered by the above teachings and are within the purview of the appended claims without departing from the spirit and intended scope of the invention. For example, while the present invention has been described in terms of systems, methods and tools for implanting a stabilization device between two vertebra, the systems, methods and tools described herein more generally may be used to implant bone stabilization devices in other locations such as an arm or leg, for example, to treat a bone fracture.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-182. (canceled)

183. A method of treating the spine, comprising:
implanting a first bone anchor assembly in a first bony element;
implanting a second bone anchor assembly in a second bony element;
providing a first access device comprising an elongate tube with a proximal end and a distal end;
inserting the first access device into the patient through a first incision;
coupling the distal end of the first access device to the first bone anchor assembly;
providing a second access device comprising an elongate tube with a proximal end and a distal end;
inserting the second access device into the patient through a second incision;
coupling the distal end of the second access device to the second bone anchor assembly;
aligning the first bone anchor assembly and the second bone anchor assembly;
inserting a connecting rod into the first access device at the proximal end;
moving the connecting rod inside the first access device from the proximal end to the distal end of the first access device;
aligning the connecting rod with the first and second bone anchor assemblies;
coupling the connecting rod to the first and second bone anchor assemblies;
uncoupling the first and second access devices from the first and second bone anchor assemblies; and
removing the first and second access devices from the patient.

184. The method of claim 183, wherein the first incision and the second incision are the same incision.

185. The method of claim 183, wherein the first incision and the second incision are different incisions.

186. The method of claim 183, wherein the first bony element comprises a first vertebrae and the second bony element comprises a second vertebrae.

187. The method of claim 183, further comprising installing a first guidewire into the first boney element and a second guide wire in the second boney element.

188. The method of claim 187, wherein at least a portion of at least one of the first bone anchor assembly and the first access device are threaded over and guided into position via the first guidewire, and wherein at least a portion of at least one of the second bone anchor assembly and the second access device are threaded over and guided into position via the second guidewire.

189. The method of claim 183, wherein aligning the first bone anchor assembly and the second bone anchor assembly comprises:
mechanically coupling the first access device to the second access device; and
aligning the first access device relative to the second access device.

190. The method of claim 189,
wherein coupling the distal end of the first access device to the first bone anchor assembly comprises substantially fixing the first access device to the first bone anchor, and
wherein coupling the distal end of the second access device to the second bone anchor assembly comprises substantially fixing the second access device to the second bone anchor.

191. The method of claim 183, further comprising pivotably coupling a first end portion of the connecting rod to a receiving portion of the first bone anchor.

192. The method of claim 191, wherein coupling the connecting rod to the first and second bone anchor assemblies comprises pivoting the connecting rod about the first end portion such that a second end portion of the connecting rod is disposed in a receiving portion of the second bone anchor assembly.

193. The method of claim 183, further comprising:
inserting a first cap member into the first access device at the proximal end;
moving the first cap member inside the first access device from the proximal end to the distal end of the first access device;
coupling the first cap member to the first bone anchor assembly to substantially inhibit pivoting of the connecting rod member relative to the first bone anchor assembly;
inserting a second cap member into the second access device at the proximal end;
moving the second cap member inside the second access device from the proximal end to the distal end of the second access device; and
coupling the second cap member to the second bone anchor assembly to substantially inhibit pivoting of the connecting rod member relative to the second bone anchor assembly.

194. The method of claim 183, further comprising:
inserting a tissue cutting tool into the first access device; and
activating the tissue cutting tool to cut a slit in subcutaneous tissue adjacent the distal end of the first access device, wherein the slit is configured to facilitate pivoting the connecting rod about the first end portion.

195. A method, comprising:
providing a first access device with a proximal portion and a distal portion and comprising an elongate access channel extending along a length of the first access device;
coupling the distal portion of the first access device to a first bone anchor assembly;
cutting a first incision proximate a first bony element;
implanting the first bone anchor assembly into the first bony element via passing of first bone anchor assembly and at least the distal portion of the first access device through the first incision such that the elongate channel provided protected access to a subcutaneous location proximate the first bony element;
inserting a connecting rod into the elongate channel of the first access device;
pivotably coupling a first end portion of the connecting rod to a rod receiving portion of the first bone anchor assembly;
pivoting the connecting rod about the first end portion such that a second end portion of the connecting rod is disposed in a receiving portion of a second bone anchor assembly disposed in a second bony structure;
uncoupling the first access devices from the first bone anchor assembly; and
removing the first access device from the patient.

196. The method of claim 195, wherein first bone anchor assembly and the second bone anchor assembly comprise a pedicle screw.

197. The method of claim 195, further comprising:
inserting a first cap member into the elongate channel of the first access device; and
coupling the first cap member to the first bone anchor assembly to substantially inhibit pivoting of the connecting rod member relative to the first bone anchor assembly.

198. The method of claim 195, wherein the distal portion of the first access device comprises tangs, and wherein coupling the distal portion of the first access device to the first bone anchor assembly comprises coupling the tangs to a complementary portion of the first bone anchor assembly.

199. The method of claim 195, further comprising:
providing a second access device with a proximal portion and a distal portion and comprising an elongate access channel extending along a length of the second access device;
coupling the distal portion of the second access device to a second bone anchor assembly;
cutting a second incision proximate a second bony element;
implanting the second bone anchor assembly into the second bony element via passing of second bone anchor assembly and at least the distal portion of the second access device through the second incision such that the elongate channel provides protected access to a subcutaneous location proximate the first bony element.

200. The method of claim 199, further comprising
mechanically coupling the first access device to the second access device; and
aligning the first access device relative to the second access device.

201. The method of claim 195, further comprising
inserting a tissue cutting tool into the elongate access channel of the first access device; and
activating the tissue cutting tool to cut a slit in subcutaneous tissue adjacent the distal portion of the first access device, wherein the slit is configured to facilitate pivoting the connecting rod about the first end portion.

202. A method comprising:
providing an access device with a proximal portion and a distal portion and comprising an elongate access channel extending along a length of the access device, wherein a distal portion of the elongate access channel comprises a longitudinally oriented opening;
coupling the distal portion of the access device to a first bone anchor assembly;
inserting a connecting rod into the elongate channel of the access device;
pivotably coupling a first end portion of the connecting rod to a rod receiving portion of the first bone anchor assembly; and
pivoting the connecting rod about the first end portion such that a second end portion of the connecting rod rotates through the longitudinally oriented opening of the first access device and is disposed in a receiving portion of a second bone anchor assembly disposed in a second bony structure.

203. The method of claim 202, further comprising
inserting a tissue cutting tool into the elongate access channel of the first access device; and
extending a blade of the tissue cutting tool through the longitudinally oriented opening of the first access device to cut a slit in subcutaneous tissue adjacent the distal portion of the first access device, wherein the slit is configure to facilitate pivoting the connecting rod about the first end portion.
